(19) United States
(12) Patent Application Publication
(10) Pub. No.: US 2011/0301095 A1
(43) Pub. Date: Dec. 8, 2011

(54) METHODS AND COMPOSITIONS FOR ASSESSING AND TREATING METASTASIS, METASTATIC CANCER, AND POTENTIAL FOR METASTASIS

(75) Inventors: Ze'ev A. Ronai, La Jolla, CA (US); Jianfei Qi, La Jolla, CA (US)

(73) Assignee: Sanford-Burnham Medical Research Institute California

(21) Appl. No.: 13/154,026

(22) Filed: Jun. 6, 2011

Related U.S. Application Data
(60) Provisional application No. 61/351,830, filed on Jun. 4, 2010.
(60) Provisional application No. 61/353,727, filed on Jun. 11, 2010.

(51) Int. Cl. A61K 38/02; G01N 33/566; G01N 21/75; G01N 21/64; C12Q 1/66; A61P 7/00; C40B 30/04; A61K 31/713; A61K 31/711; A61P 35/00; A61P 35/04; C12Q 1/04; C12Q 1/68

(52) U.S. Cl. 514/19.5; 435/34; 436/501; 435/7.9; 435/8; 435/6.12; 506/9; 514/19.3; 514/44 A; 514/44 R; 514/19.8

(57) ABSTRACT

Disclosed are methods, compounds, and compositions for detection, diagnosis, prognosis, monitoring, treatment, monitoring treatment, and selecting treatment of cancer and metastasis, and for identifying compounds and compositions for such uses. For example, disclosed are methods, compounds, and compositions for detecting a risk of metastasis of cancer in a subject, treating a subject at risk of metastasis of cancer, identifying an inhibitor of HIF1α:FoxA2 function or complex formation, detecting a risk of neuroendocrine differentiation (NED)-associated cancer, determining a prognosis of a cancer, determining a treatment for a cancer, monitoring or determining the effect of treatment of a NED-associated cancer, treating NED-associated cancer, identifying an inhibitor of HIF1α:FoxA2 complex formation, detecting neuroendocrine differentiation (NED)-associated cancer, monitoring the risk of metastasis of cancer in a subject, and treating NED-associated cancer.

### CROSS-REFERENCE TO RELATED APPLICATIONS

This application claims benefit of U.S. Provisional Application No. 61/351,830, filed Jun. 4, 2010, and U.S. Provisional Application No. 61/353,727, filed Jun. 11, 2010. Application No. 61/351,830, filed Jun. 4, 2010, and Application No. 61/353,727, filed Jun. 11, 2010, are hereby incorporated herein by reference in their entirety.

### STATEMENT REGARDING FEDERALLY SPONSORED RESEARCH

This invention was made with government support under grants CA111515, U01 CA084294, and P50CA090386 awarded by the National Cancer Institute (NCI) of the National Institutes of Health (NIH). The government has certain rights in the invention.

### REFERENCE TO SEQUENCE LISTING

The Sequence Listing submitted Jun. 6, 2011 as a text file named “SBMRI_{—}49_{—}8403 AMD_AFD_Sequence_Listing.TXT,” created on Jun. 6, 2011, and having a size of 10,504 bytes is hereby incorporated by reference pursuant to 37 C.F.R. §1.52(e)(5).

### FIELD OF THE INVENTION

The disclosed invention is generally in the field of cancer and cancer treatment and specifically in the area of detection, diagnosis, prognosis, monitoring, treatment, monitoring treatment, and selecting treatment of cancer and metastasis.

### BACKGROUND OF THE INVENTION

Prostate cancer is the second leading cause of cancer deaths among men in Western nations. Among the metastatic forms of prostate adenocarcinoma (PCa) are those that express neuroendocrine (NE) markers, often referred to as neuroendocrine differentiation (NED) or NE phenotype. NED is seen in over 30% of PCa and is associated with poor prognosis and androgen independence (Cindolo et al., 2007). A small percentage (0.5-2%) of human prostate tumors develops as highly aggressive NE tumors, which have a 35% survival rate in 2 years (Cindolo et al., 2007; Sella et al., 2000). Factors implicated in NED of LNCaP prostate cancer cells in vitro include IL-6 treatment (Deeble et al., 2001), androgen removal (Yuan et al., 2006) and ionizing radiation (Deng et al., 2008). In transgenic animals, T antigen expression or inactivation of p53 and Rb has been associated with prostate NE tumors (Huss et al., 2007; Zhou et al., 2006).

FoxA2, a member of the FoxA subfamily of forkhead box transcription factor, is expressed in mouse prostate NE carcinomas (Chiaverotti et al., 2008; Mirosevich et al., 2006) and NE foci of human PCa (Mirosevich et al., 2006). HIF-1α, the master regulator of the hypoxia response, is also expressed in NE tumors (Monsef et al., 2007). HIF-1α is regulated under normoxia by the E3 ligase pVHL and is also regulated under mild hypoxia (2-5% O2) by the ubiquitin ligase Siah2. Siah2 controls prolyl hydroxylase ⅓ (PHD) stability (Nakayama et al., 2004), thereby affecting PHD availability to modify HIF-1α, which is essential for HIF-1α's association with and ubiquitination by pVHL (Ivan et al. 2001). Given that PHD function as cellular oxygen sensors (Aragones et al., 2009; Nakayama et al., 2009), Siah2 is expected to play a central role in controlling hypoxia and related biological outcomes, including tumorigenesis and metastasis (Nakayama et al., 2009). Indeed, inhibition of Siah2 activity blocks formation of tumors (Ahmed et al., 2008; Moller et al., 2009; Qi et al., 2008; Schmidt et al., 2007). Further, Siah2′s contribution to melanoma metastasis is HIF-dependent (Qi et al., 2008).

Once stabilized, HIF-1α translocates to the nucleus and dimerizes with HIF-1β, the heterodimers then bind to hypoxia responsive elements (HREs) to regulate transcription of hypoxia-responsive genes (Semenza, 2003). Several transcription factors cooperate with HIF to regulate its transcriptional activity, HIF activity is enhanced by β-catenin (Kaidi et al., 2007) and repressed by FOXO3a (Emerling et al., 2008). HIF can also modulate activity of other transcriptional regulators: HIF-1α potentiates Notch signaling (Gustafsson et al., 2005) and represses c-Myc activity (Gordan et al., 2007).

### BRIEF SUMMARY OF THE INVENTION

Disclosed are methods, compounds, and compositions for detecting, diagnosing, determining the prognosis, monitoring, treatment, monitoring treatment, and selecting treatment of cancer and metastasis. The disclosed methods, compounds, and compositions are particularly suited for neuroendocrine differentiation (NED)-associated cancers and cancers that could develop neuroendocrine phenotype.

For example, disclosed are methods, compounds, and compositions for detecting the presence of or a risk of metastasis of cancer in a subject. Such methods can comprise detecting one or more cells in a cancer sample from the subject that express FoxA2 and HIF-1α, wherein detection of cells that express FoxA2 and HIF-1α indicate the presence of or a risk of metastasis of cancer in the subject. Also disclosed are methods, compounds, and compositions of detecting the presence of or a risk of neuroendocrine differentiation (NED)-associated cancer. Such methods can comprise detecting one or more cells in a sample that express FoxA2 and HIF-1α, wherein detection of cells that express FoxA2 and HIF-1α indicate the presence of or a risk of NED-associated cancer.

Also disclosed are methods, compounds, and compositions of monitoring the risk of metastasis of cancer in a subject. Such methods can comprise detecting one or more cells in a cancer sample that express FoxA2 and HIF-1α, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has reduced the risk of metastasis in the subject. The cancer sample can be a treated cancer sample, wherein the treated cancer sample can be from a subject with cancer that has been treated.

Also disclosed are methods, compounds, and compositions of detecting neuroendocrine differentiation (NED)-associated cancer. Such methods can comprise detecting one or more cells in a cancer sample that express FoxA2 and HIF-1α, wherein detection of cells that express FoxA2 and HIF-1α indicate NED-associated cancer.

Also disclosed are methods, compounds, and compositions of determining a prognosis of a cancer. Such methods can comprise detecting one or more cells in a cancer sample that express FoxA2 and HIF-1α, wherein detection of cells that express FoxA2 and HIF-1α indicate a poor prognosis of the cancer.

Also disclosed are methods, compounds, and compositions of identifying an inhibitor of HIF1α:FoxA2 function or complex formation. Such methods can comprise contacting a compound with HIF-1α or FoxA2; assaying binding of the compound to HIF-1α or FoxA2; and determining if the compound inhibits HIF-1α:FoxA2 function or complex formation. Also disclosed are methods, compounds, and compositions of identifying an inhibitor of HIF1α:FoxA2 complex formation. Such methods can comprise producing a peptide having at least 85% sequence identity to the HIF-1α:FoxA2 interaction domain, and assaying the peptide for the ability to inhibit the formation of a HIF-1α:FoxA2 complex.

Also disclosed are methods, compounds, and compositions of determining a treatment for a cancer. Such methods can comprise detecting one or more cells in a sample that express FoxA2 and HIF-1α, wherein detection of cells that express FoxA2 and HIF-1α indicate as the treatment a NED-associated cancer treatment.

Also disclosed are methods, compounds, and compositions of treating a subject at risk of metastasis of cancer. Such methods can comprise administering to a subject a composition that inhibits expression of one or more HIF-1α:FoxA2-regulated genes. Also disclosed are methods, compounds, and compositions of treating NED-associated cancer. Such methods can comprise administering to a subject a composition that can inhibit the formation of a HIF-1α:FoxA2 complex. Also disclosed are methods, compounds, and compositions of treating NED-associated cancer. Such methods can comprise administering to a subject a composition that inhibits expression of one or more HIF-1α:FoxA2-regulated genes.

Also disclosed are methods, compounds, and compositions of monitoring or determining the effect of treatment of a NED-associated cancer. Such methods can comprise detecting one or more cells in a cancer sample that express FoxA2 and HIF-1α, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has had a positive effect. The cancer sample can be a treated cancer sample, wherein the treated cancer sample can be from a subject with a NED-associated cancer that has been treated,

In some forms of the disclosed methods, FoxA2 and HIF-1α can both be present at or above respective reference levels in the cells that express FoxA2 and HIF-1α, where the presence of FoxA2 and HIF-1α at or above the respective reference levels indicate the presence of or a risk of metastasis of cancer in the subject, the presence of or a risk of neuroendocrine differentiation (NED)-associated cancer, or a combination. In some forms of the disclosed methods, FoxA2 and HIF-1α can both be present at or above respective reference levels in the cells that express FoxA2 and HIF-1α, where the presence of FoxA2 and HIF-1α at or above the respective reference levels indicate the presence of or a risk of metastasis of cancer in the subject. In some forms of the disclosed methods, FoxA2 and HIF-1α can both be present at or above respective reference levels in the cells that express FoxA2 and HIF-1α, where the presence of FoxA2 and HIF-1α at or above the respective reference levels indicates NED-associated cancer. In some forms of the disclosed methods, detection of cells that express FoxA2 and HIF-1α indicates a poor prognosis of the cancer.

In some forms, the disclosed methods can further comprise determining if one or more of the cells that express FoxA2 and HIF-1α also express Hes6, Sox9, Jmjd1a, Plod2, or a combination. In some forms, Hes6, Sox9, Jmjd1a, Plod2, or a combination can be present at or above respective reference levels in the cells that express Hes6, Sox9, Jmjd1a, Plod2, or a combination.

In some forms, the cancer sample can be a prostate sample, a lung sample, a pancreatic sample, or a merkel cell sample. In some forms of the disclosed methods, the cancer can be prostate cancer, lung cancer, pancreatic cancer, or merkel cell carcinoma.

In some forms, the disclosed methods can further comprise treating the subject with a cancer treatment. In some forms, the cancer treatment can be a neuroendocrine differentiation (NED)-associated cancer treatment. In some forms, the subject can have prostate cancer, lung cancer, pancreatic cancer, or merkel cell carcinoma.

In some forms, the composition can comprise an inhibitor of HIF-1α. In some forms, the inhibitor of HIF-1α can comprise a Siah2 inhibitor. In some forms, the Siah2 inhibitor can be a PHYL peptide. In some forms, the composition can comprise an inhibitor of FoxA2. In some forms, the inhibitor of FoxA2 can be shRNA. In some forms, the composition can disrupt formation of HIF-1α:FoxA2 complex. In some forms, the composition can inhibit the formation of a HIF-1α:FoxA2 complex. In some forms, the HIF-1α:FoxA2 complex can comprise HIF-1α:FoxA2 interaction domain, where the composition competes for the HIF-1α:FoxA2 interaction domain.

In some forms, the composition can reduce p300 recruitment. In some forms, the composition can inhibit p300. In some forms, the HIF-1α:FoxA2-regulated genes can be Hes6, Sox9, Jmjd1a, Plod2, or a combination. In some forms, the composition can comprise a compound and a pharmaceutically acceptable carrier. In some forms, the composition can comprise two or more different inhibitors of expression of HIF-1α:FoxA2-regulated genes. In some forms, the composition can comprise a vector. In some forms, the compound can be a peptide having at least 85% sequence identity to HIF-1α:FoxA2 interaction domain.

In some forms, the subject can be diagnosed with a NED-associated cancer prior to treatment. In some forms, the subject can have suffered from cellular hypoxia. In some forms, the cellular hypoxia can be mild cellular hypoxia. In some forms, the cancer can comprise one or more cells that express FoxA2 and HIF-1α.

In some forms of the disclosed methods, the presence of or the risk of metastasis can be indicated by one or more cancer cells that express FoxA2 and HIF-1α. In some forms of the disclosed methods, the presence of or the risk of metastasis can be indicated by one or more of the cancer cells that express FoxA2 and HIF-1α also expressing Hes6, Sox9, Jmjd1a, Plod2, or a combination.

In some forms, the NED-associated cancer can comprise one or more cells that express FoxA2 and HIF-1α.

In some forms, the subject can have a risk of metastasis of the NED-associated cancer. In some forms, the risk of metastasis can be indicated by one or more cancer cells that express FoxA2 and HIF-1α. In some forms, the risk of metastasis can be indicated by one or more of the cancer cells that express FoxA2 and HIF-1α also expressing Hes6, Sox9, Jmjd1a, Plod2, or a combination.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1E show tumorigenesis in TRAMP^{Tg}/Siah2 mice. A. Primary tumor incidences of TRAMP mice with indicated Siah2 genotype are shown as percentages. B. Re-expression of HIF-1α in PHYL-expressing TRAMP-C cells. Cells were stably transfected with indicated expression vectors. PHYL-expressing cells were further stably transfected with HIF-1α and maintained in normoxia (N) or hypoxia (H) for 6 h before analyses by western blot for HIF-1α. C. Incidence of NE and AH in TRAMP mice with indicated Siah genotypes. Number of mice for each genotype is indicated. Dark bars indicate NE+AH. p<0.005 for NE tumor incidence between control and Siah-deficient TRAMP mice. . D. and E. TRAMP-C cells (3×10⁶) expressing control pKH3 vector, PHYL or PHYL+HIF-1α were injected subcutaneously into the flanks of nude mice. The frequency of tumor formation (D) and size of xenograft tumor (E) 8-weeks post injection are shown. In panel E, each column represents mean±SD (Standard Deviation) for 5 mice, p<0.05 for pKH3 vs. PHYL+HIF-1α. See also FIG. 8 and Table 1.

FIG. 2 shows metastasis in TRAMP^{Tg}/Siah2 mice. Percentage of metastases in TRAMP mice with indicated Siah genotypes. Number of mice and tissues examined: N=32 (S2^{+/+} and S2^{+/+}), or 9 (S1a^{+/-};S2^{-/-}), for liver and lung, and N=26 (S2^{+/+} or S2^{+/-}), 11 (S2^{-/-}), or 6 (S1a^{+/-}:S2^{+/-}) for lymph nodes. For each case, 5 serial sections of lungs or liver were examined by H&E staining The first bar in each group represents S2^{+/-} and S2^{+/-}, the middle bar in each group represents S2^{-/-}, and the last bar in each group represents S1a^{+/+}:S2^{+/-}

FIGS. 3A-3K show FoxA2 enhances HIF transcriptional activity. A. TRAMP-C cells were transfected with an HRE-Luc construct and the indicated plasmids. 24-h post transfection, cells were maintained in 1% oxygen for 10 h, and cell lysates were collected to measure luciferase activity. β-Gal plasmid was used to normalize for transfection efficiency. N and H denote normoxia and hypoxia, respectively. p<0.01 pcDNA (N) vs. HIF (N), p<0.0005 HIF (N) vs. HIF+FoxA2 (N). B. TRAMP-C cells were first transfected with indicated siRNAs then 48-h later with an HRE-luc construct. 24 h after the second transfection, cells were maintained in 1% oxygen for 10 h before analysis of luciferase activity. p<0.005 for Control (H) vs. HIF-1α (H), HIF-1β (H), or FoxA2 (H). C. TRAMP-C cells were transfected with the FOXA-Luc construct and indicated FoxA2 deletion mutants. 24-h post transfection, cells were treated with 1% oxygen for 10 h before analysis of luciferase activity. p<0.01 WT vs. M-1, p>0.1 WT vs. M-2 or M-3. D. TRAMP-C cells were transfected with an HRE-Luc construct and the indicated plasmids. 24-h post transfection, cells were maintained in 1% oxygen for 10 h before analysis of luciferase activity. p<0.001 HIF+WT (N) vs. HIF+m3 (N). Each column in panels A-D represents mean±SD of 3 replicates. E. 293T cells were transfected with indicated plasmids. HIF-1α was precipitated with Flag antibody-conjugated beads (M2) 48-h post transfection and the precipitated proteins were analyzed by immunoblot. F. TRAMP-C cells were maintained in 1% O₂ for 5 h. Endogenous HIF-1α was precipitated and co-precipitated proteins were analyzed by immunoblot. G. TRAMP-C cells were transfected with HIF-1α siRNA for 48 h then maintained in hypoxia for 5 h. Endogenous HIF-1β was precipitated and co-precipitated proteins were analyzed by immunoblot. H. HIF-1α and its truncation mutants were translated in vitro, labeled with ³⁵S, and incubated with Nickel beads coated with His-FoxA2. After 3 washes, proteins on the beads were separated by SDS-PAGE and transferred to nitrocellulose membrane. ³⁵5-labeled HIF-1α was detected by phosphor-imager, followed by immunoblot with a His antibody to detect His-FoxA2. 4% of in vitro translated ³⁵5-HIF-1α was used as input. I. Flag-HIF-1α was translated in vitro and bound to the M2 beads then incubated with ³⁵S-labeled in vitro translated FoxA2 or its truncation mutants. Bound proteins were monitored as indicated in panel H. J. TRAMP-C cells were stably transfected with indicated vectors and then grown in 1% O₂ for 6 h before immunoprecipitation of HIF-1α. The co-precipitated proteins were analyzed by western blot. K. Flag-HIF-1α was translated in vitro and bound to the M2 beads then incubated with ³⁵S-labeled in vitro translated FoxA2 and HIF-113. Bound proteins were monitored as indicated in panel H. See also FIG. 9.

FIGS. 4A-4L demonstrate a subset of HIF target genes are regulated by cooperation with FoxA2. A. TRAMP-C cells were transfected with FoxA2 siRNAs. 48-h post transfection, cells were maintained in 1% oxygen for 10 h then RNA was isolated for qRT-PCR analyses of the indicated transcripts. Control (H) vs. FoxA2 (H): p>0.1 for VEGFA and Glut-1, p<0.005 for all others. The first bar in each group represents Control siRNA(N), the second bar in each group represents Control siRNA(H), the third bar in each group represents FoxA2 siRNA(N), and the fourth bar in each group represents FoxA2 siRNA(H). B. TRAMP-C cells were transfected with indicated siRNAs. 48-h post transfection, cells were maintained in 1% oxygen for 10 h. RNA was isolated for qRT-PCR analyses of the

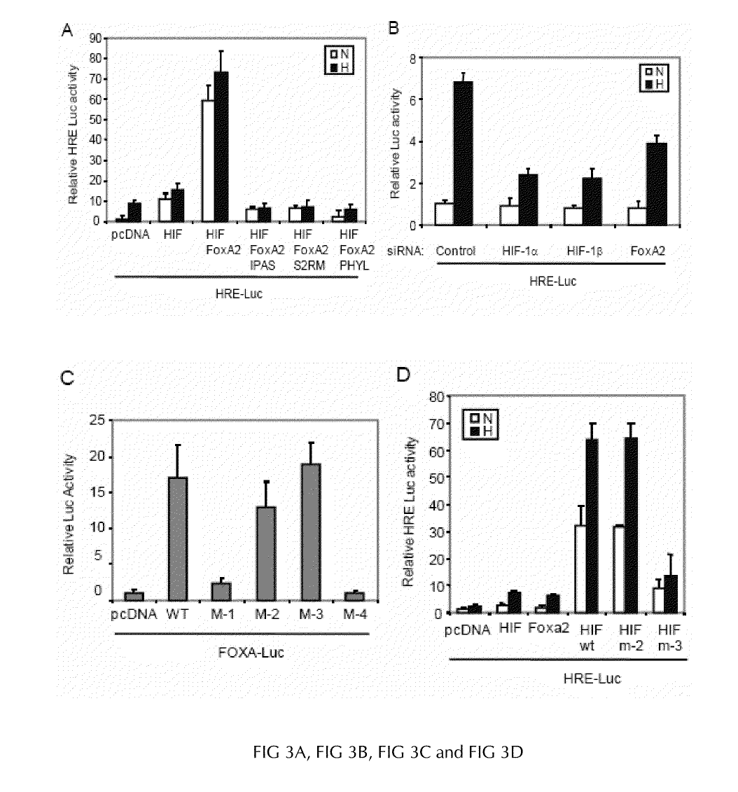

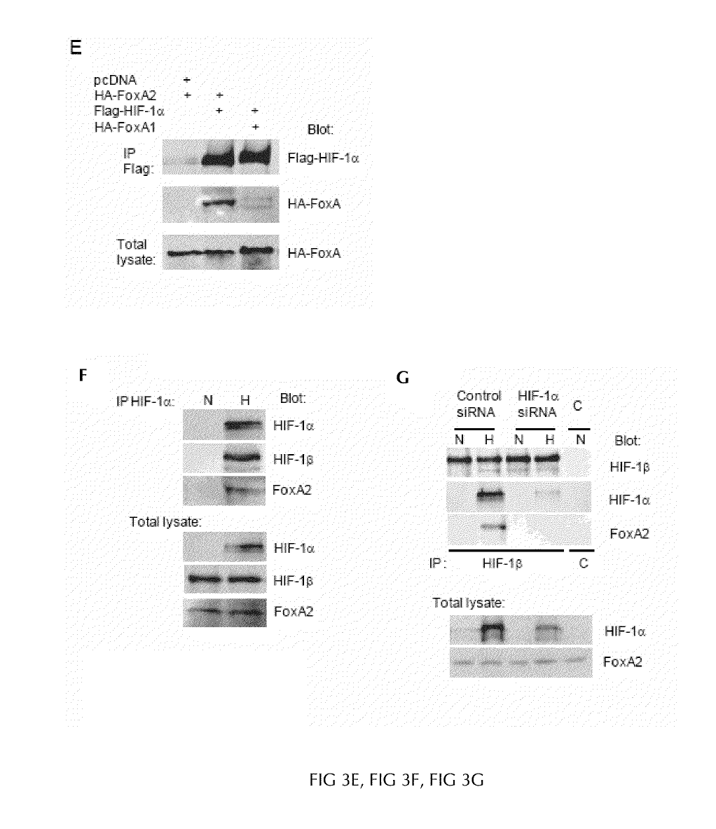

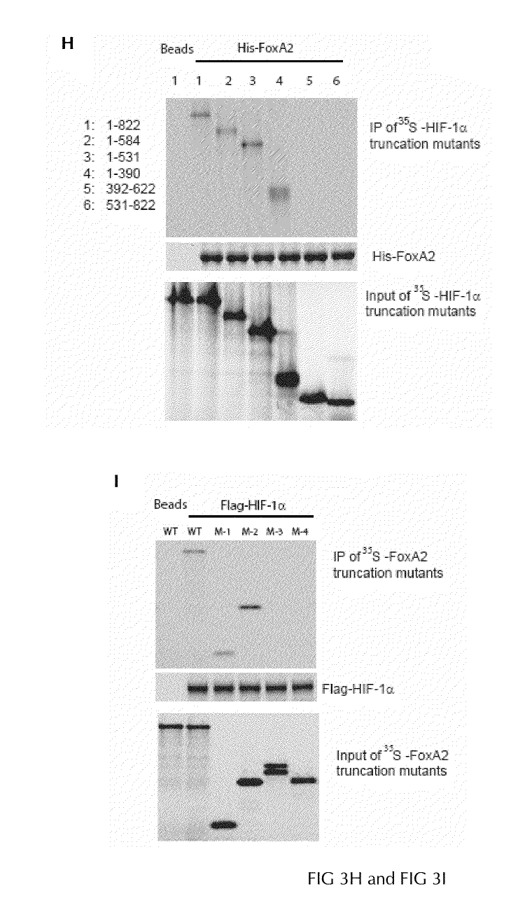

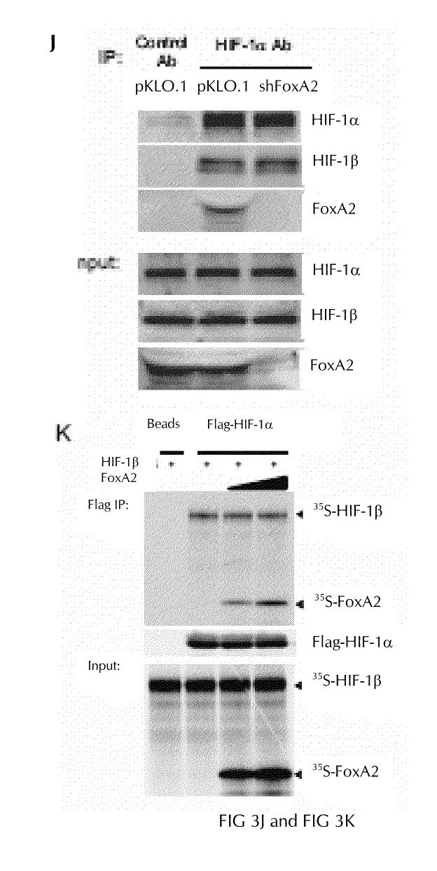

Hes6 transcript. p<0.05 between control (H) and HIF-1α (H), or FoxA2 (H). C. TRAMP-C cells were transfected with a 1.25 kb Hes6 promoter-Luc construct containing the wild type or mutated -66 by HRE. 24-h post transfection, cells were treated with DMOG (1 mM, 16 h) before analysis of luciferase activity. p<0.005 for WT Hes6−DMOG vs. +DMOG, p>0.1 for mutant Hes6−DMOG vs. +DMOG. D. TRAMP-C cells were transfected with indicated plasmids. 24-h post transfection, cells were treated with DMOG (1 mM, 16 h) before analysis of luciferase activity. WT Hes6: p<0.01 pcDNA−DMGO vs. pcDNA+DMOG, p<0.05 pcDNA+DMGO vs. Foxa2+DMOG. E. TRAMP-C cells were transfected with control or FoxA2 siRNA. 48-h post transfection, cells were grown in 1% oxygen for 5 h before ChIP assays of the HRE-containing region of Hes6 promoter were performed using indicated antibodies. PCR products were analyzed on 2% agarose gel electrophoresis. A representative reversed gel image of triplicate experiments is shown. F. FoxA2 shRNA-expressing cells were treated with 1% oxygen for 6 h and subjected to ChIP assays with p300 antibodies. The immunoprecipitated materials were used for QPCR analyses of the HRE-containing regions of VEGFA, Jmjd1a and Hes6. The results of ChIP QPCR were normalized to those of the input. p<0.01 for all genes between control and shFoxA2 for both cell lines. G. Cells were transfected with control or p300 siRNA for 48 h then analyzed by qRT-PCR for the indicated transcripts. The first bar in each group represents control shRNA. The second bar in each group represents p300 shRNA. Both TRAMP-C and Rv1: p>0.1 control vs. p300 for VEGFA and p<0.05 for all others. H. TRAMP-C cells were co-transfected with Hes6 or VEGFA promoter-Luc vector, HIF-1α, and increasing amounts of p300. 24-h post transfection, cell lysates were collected for a luciferase assay. Hes6: p<0.001 0 μg vs. all three; VEGFA: p<0.005 0 μg vs. 0.5 μg, p>0.1 0 μg vs. the other two. I and J. TRAMP-C cells were transfected with Hes6 promoter-Luc (H) or VEGFA promoter-Luc (I) together with the plasmids indicated. 24-h post transfection, cell lysates were collected for analysis of luciferase activity. In panels A-D, F-J, each column represents mean±SD of 3 experiments. Hes6: p<0.01 HIF vs. HIF+FoxA2, HIF vs. HIF+p300, and HIF+FoxA2 vs. HIF+FoxA2+p300; VEGFA: p>0.1 for these comparisons. K. Flag-p300 was translated in vitro and coupled to M2 beads. HIF-1α or FoxA2 was translated in vitro and labeled with ³⁵S. Equal amounts of ³S-HIF-1α and ³⁵S-FoxA2 were incubated with M2 bead-bound Flag-p300. Bound proteins were monitored as indicated in FIG. 3H. L. Flag-p300 (wt or ACH1) was translated in vitro and bound to M2 beads. HIF-1α and FoxA2 were translated in vitro and labeled with ³⁵S. Equal amount of ³⁵S-HIF-1α and ³⁵S-FoxA2 was mixed and incubated with M2 bead-bound Flag-p300 or Flag-p300ACH1. Bound proteins were monitored as indicated in FIG. 3H. See also FIG. 10, Tables 2, 3, and 4.

FIGS. 5A-5F demonstrate Hes6, Sox9 and Jmjd1a are required for tumorigenesis of TRAMP cells. A. TRAMP-C cells were stably transfected with indicated vectors. Inset shows western blot of PHYL and N×N. PHYL-expressing cells were then infected with retroviral constructs encoding Hes6, Sox9 or Jmjdl a individually or all together (HSJ). lx10⁵ cells were monitored for growth on soft agar under 1% O2 for 3 weeks. Shown is the number of colonies per well of 6-well plate. p=0.4 (pBabe vs N×N), p=0.053 (N×N vs. PHYL+HSJ), p<0.0005 (N×N vs. PHYL, PHYL+Hes6, PHYL+Sox9, or PHYL+Jmjd1a), p<0.05 (PHYL+HSJ vs. PHYL, PHYL+Hes6, PHYL+Sox9, or PHYL+Jmjd1a). B. TRAMP-C cells were transfected with indicated shRNA. Inset shows western blot of FoxA2. shFoxA2-expressing cells were then infected with retroviral constructs of Hes6, Sox9 or Jmjd1a individually or all together (HSJ). lx10⁵ cells were monitored for their growth on soft agar under 1% O₂ for 3 weeks. Shown is the number of colonies per well of 6-well plate. In panel A and B, each column represents mean±SD for 3 replicates. p=0.06 (pKLO.1 vs. shFoxA2+HSJ), p<0.005 (pKLO.1 vs. shFoxA2, shFoxA2+Hes6, shFoxA2+Sox9, or shFoxA2+Jmjd1a), p<0.005 (shFoxA2+HSJ vs. shFoxA2, shFoxA2+Hes6, shFoxA2+Sox9, or shFoxA2+Jmjd1a). C, D, E, F. lx10⁶ of TRAMP-C transfectants as described in FIG. 5A and 5B were injected into the prostate of nude mice. Two months after injection, genitourinary tracts of mice were dissected and prostate tumor formation was quantified. Panels D and F depict the frequency of tumor formation. Panels E and C show the average size of the tumors formed. In panels E and F, each column represents mean±SD for 5 mice. p<0.05 pBabe vs. PHYL+HSJ and N×N vs. PHYL+HSJ, p<0.001 (pKLO.1 vs. shFoxA2, shFoxA2+Hes6, or shFoxA2+Jmjd1a), p<0.005 (shFoxA2+HSJ vs. shFoxA2, shFoxA2+Hes6, or shFoxA2+Jmjd1a), p<0.01 (pKLO.l vs. shFoxA2+Sox9), p=0.2 (pKLO.1 vs. shFoxA2+HSJ). See also FIG. 11.

FIGS. 6A-6I shows hypoxia-induced NE phenotype in human prostate cancer cells. A. CWR22Rv1 cells were cultured under normoxia or hypoxia (1% O2) for indicated times before qRT-PCR analysis of NSE and ChgB. Transcript levels under hypoxia were normalized to those under normoxia. p<0.05, p<0.005, p<0.0001 for NSE hypoxia vs.

normoxia at days 1, 3 and 5, respectively. p=0.19, p<0.005, p<0.0001 for ChgB hypoxia vs. normoxia at days 1, 3 and 5, respectively. The first bar in each group represents NSE and the second bar in each group represents ChgB. B. Rv1 cells were grown under 1% O2 for indicated times. The total lysates were used for western blot analyses of NSE and ChgB. Hes6 and Sox9 were immunoprecipitated followed by western blot analysis. C. Lymph nodes (LN) were collected from the mice described in H, and stained with H & E to determine the metastases. p<0.05 (pBabe vs. PHYL), p=0.17 (PHYL vs. PHYL+HSJ), p<0.01 (pKLO.1 vs. shFoxA2), p<0.05 (shFoxA2 vs. shFoxA2+HSJ). D. Rv1cells were cultured under hypoxia for indicated times before qRT-PCR analysis. The transcript level under hypoxia was normalized to that of corresponding normoxia samples. P<0.005 for all three transcripts at all three time points. The first bar in each group represents H1 day, the middle /bar in each group represents H3 day and the last bar in each group represents H5 day. E. Rv1 cells were stably transfected with control pBabe vector or PHYL. Inset: western blot shows expression of PHYL. PHYL-expressing Rv1 cells were further infected with viral constructs of Hes6, Sox9 or Jmjd1a either individually or in combination (HSJ). Cells were cultured under 1% O2 for 5 days before qRT-PCR analysis of NSE. p<0.01 pBabe vs. PHYL+HSJ, P<0.0001 pBabe vs. all others, p<0.005 PHYL+HSJ vs. other PHYL-expressing cells. F. Rv1 cells were transfected with control pKLO.1 vector or FoxA2 shRNA. Inset: western blot shows the knockdown of FoxA2. shFoxA2-expressing cells were further infected with viral constructs of Hes6, Sox9 or Jmjd1a either individually or in combination (HSJ). Cells were cultured under 1% O2 for 5 days before qRT-PCR analysis of NSE. p<0.05 pKLO.1 vs. shFoxA2+HSJ, p<0.0001 pKLO.1 vs. all others, p<0.001 shFoxA2+HSJ vs. other shFoxA2-expressing cells. G. Rv1 transfectants were seeded on tissue culture plates at low density and maintained at 1% O2 for 6 days. The morphology of cells was examined under phase-contrast microscopy. The number of colonies with neurite-like structures (criteria: >1/3 of cells in the periphery of colonies have neurite-like structure that is over 20 um long) were scored at triplicate of 6-well plates. p<0.01 pBabe vs. PHYL or PHYL+HSJ, p<0.001 PHYL+HSJ vs. PHYL, p<0.05 pKLO.1 vs. shFoxA2 or shFoxA2+HSJ, p<0.005 shFoxA2 vs. shFoxA2+HSJ. In panel A, D, E, F, G, each column represents mean±SD for 3 replicates. H. lx10⁶ Rv1 transfectants as described in E and F were injected into the prostates of nude mice. 4-week-post injection, the allograft tumors were collected and size measured. p<0.05 pBabe vs. PHYL or PHYL+HSJ, p>0.1 PHYL vs. PHYL+HSJ and pKLO.l vs. shFoxA2 or shFoxA2+HSJ. I. Blood was collected from the heart of mice described in H before the sacrifice, cultured in the selection medium for 2 weeks. The number of colonies on the plates was scored and normalized to the volume of blood. p<0.01 pBabe vs. PHYL or PHYL+HSJ, p<0.001 PHYL vs. PHYL+HSJ, p<0.05 pKLO.1 vs. shFoxA2 or shFoxA2+HSJ, p<0.005 shFoxA2 vs. shFoxA2+HSJ. In panel H and I, each column represents mean±SD for 5 mice. See also FIG. 12, Table 5.

FIGS. 7A, 7B, and 7C show changes in HIF/FoxA2 targets in prostate tumors. A. Laser capture microdissection was performed to collect NE carcinoma cells from tumors of TRAMP/Siah2^{+/-} or TRAMP/Siah2^{-/-} mice. RNA was isolated for qRT-PCR analyses of indicated transcripts. Each column represents mean±SD for 2 replicates. Siah2^{+/-} vs. Siah2^{-/-} p<0.05 for Hes6, Sox9 and Jmjd1a, p=0.78 and 0.46 for VEGFA and Glut-1, respectively. The first bar in each group represents Siah2^{+/-} and the second bar in each group represents Siah2^{-/-}. B. IHC staining of the indicated proteins was performed on a human prostate TMA consisting PINs and prostate cancers of various Gleason scores, the numbers of each tumor group are shown on the figure. The staining intensity is scored according to 4 scales by 2 pathologists: 0 (no staining), 1 (weak staining), 2 (medium staining) and 3 (strong staining) Scale 0 and 1 are defined as negative staining, while scale 2 and 3 are defined as positive staining Shown is the percentage of cores that are positively stained for indicated antibodies. The first bar in each group represents PIN, the second bar represents G3, the third bar represents G4 and the fourth bar represents G5. C. IHC staining using the indicated antibodies were performed on serial sections of 15 human PCa specimens, among which 10 cases have NE phenotype (NSE positive) and 5 cases have no NE phenotype (NSE negative). Co-expression of Hes6, Sox9 and Jmjd1a in human PCa with NE phenotype is statistically significant compared with that in human PCa without NE phenotype (p<0.05). D. Shown are clustered patterns of gene expression taken from GSE3325. Columns represent tumor samples (Benign, B1-B6; Primary, P1-P7; Metastatic, M1-M6), and rows represent genes. Expression data for each gene was row normalized. Transcript levels of Siah2, FoxA2, Hes6, Jmjd1a, Plod2, DDC, ChgB, and ENO2 in metastatic PCa are statistically significantly higher than those in primary PCa. See also FIG. 13.

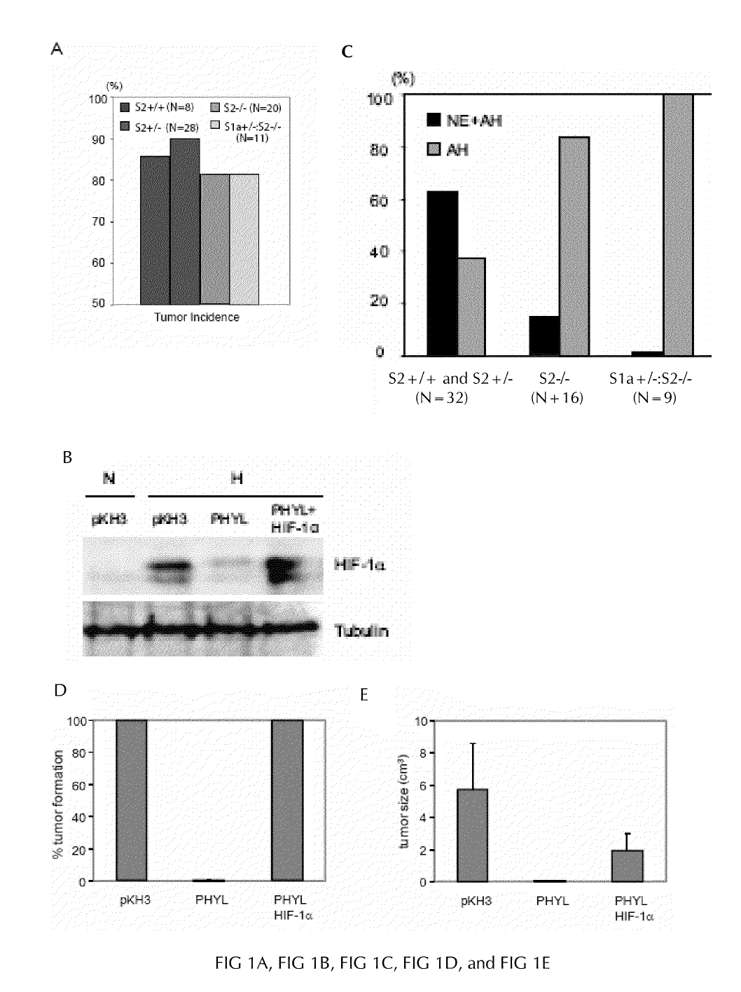

FIG. 8 (related to FIG. 1) Dorsal prostate lobes were dissected from 1- or 3-month old TRAMP mice for histological analyses. Based on morphology, AH progression was divided into 3 stages: early (hyperplasia is less than 50% of a prostate gland), medium (hyperplasia is over 50% of a prostate gland), and late (branching and expansion of hyperplasia). The percentage of normal prostate gland, early stage AH, medium stage AH and late stage AH was scored on the sections of dorsal prostate lobes from 1- or 3- month old TRAMP mice with indicated genotypes. The first bar in each group represents S2^{+/-} TRAMP and the second bar in each group represents S2^{-/-} TRAMP. Each column represents mean±SD for 5 mice.

FIGS. 9A, 9B, and 9C (related to FIG. 3) A. Knockdown of HIF-1α, HIF-1β and FoxA2. TRAMP-C cells were transfected with siRNAs of HIF-1α, HIF-1β or FoxA2. 72-h post transfection, cell lysates were collected and immunoblot analyses were performed for the indicated proteins. B. Left panel: Diagram of FoxA2 mutants. Wild type of FoxA2 consists of 3 domains (N-TAD, forkhead domain and C-TAD). FoxA2 mutants lack one or two of these domains. The FoxA2 mutants were subcloned into pcDNA3 vector with an N-terminal myc Tag. Right panel: Expression of FoxA2 mutant proteins. TRAMP-C cells were transfected with Myc-tagged wild type or mutant FoxA2. 48-h post transfection, cell lysates were collected for immunoblot analysis using myc antibody. C. Diagram of HIF-1α truncation mutants. The domains of HIF-1α in each truncation mutant were shown.

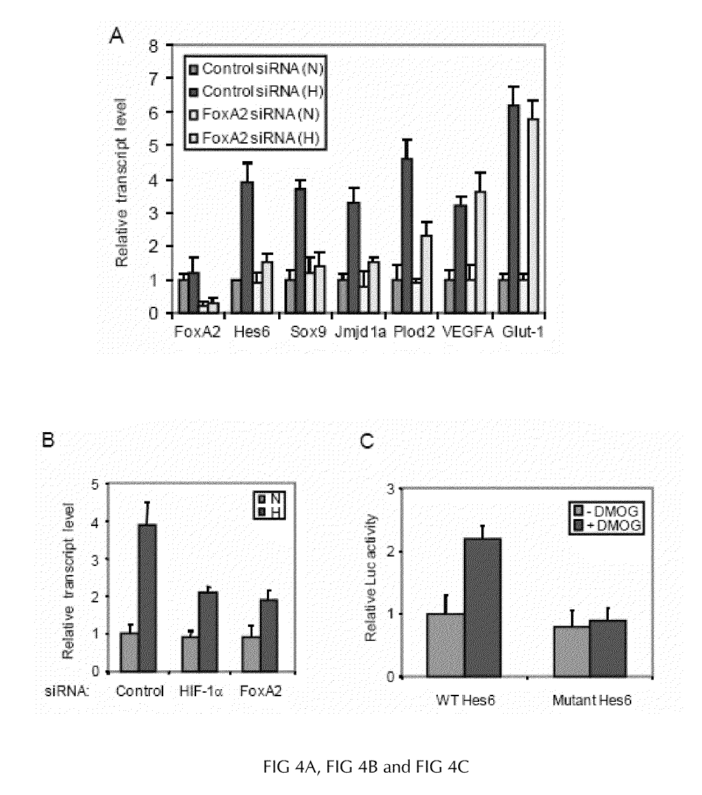

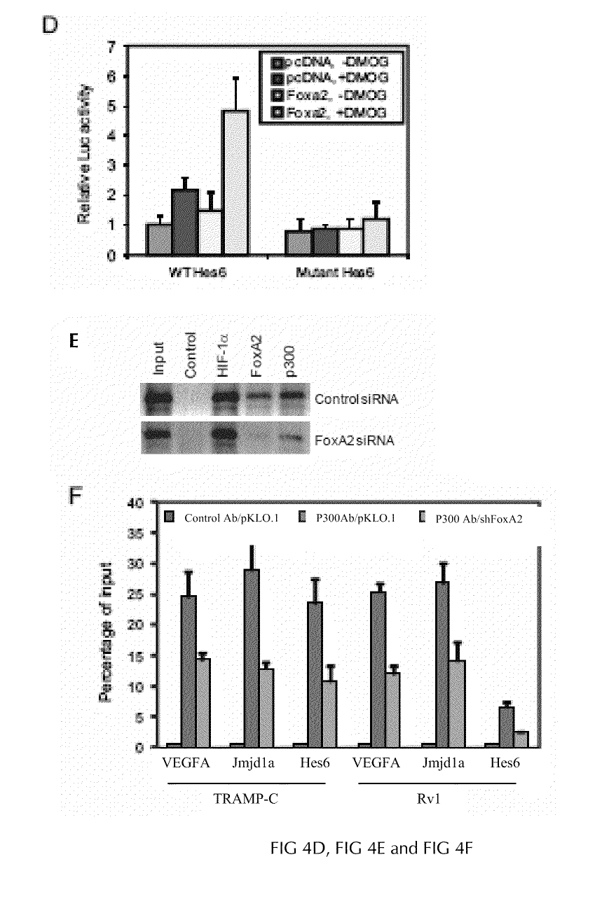

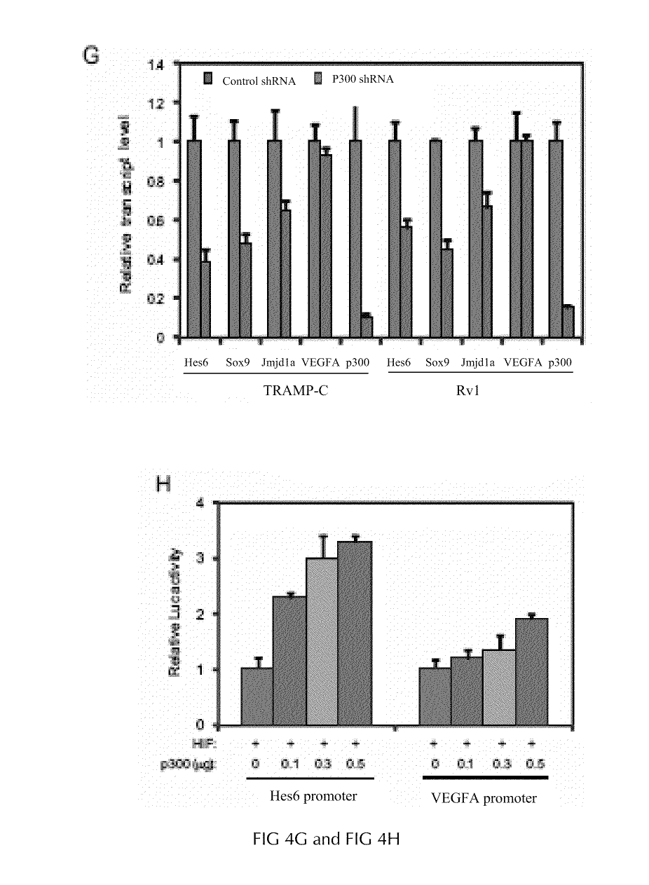

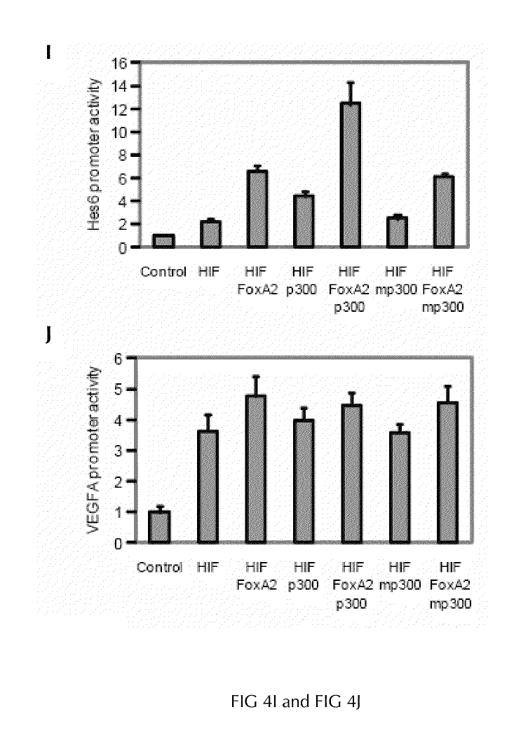

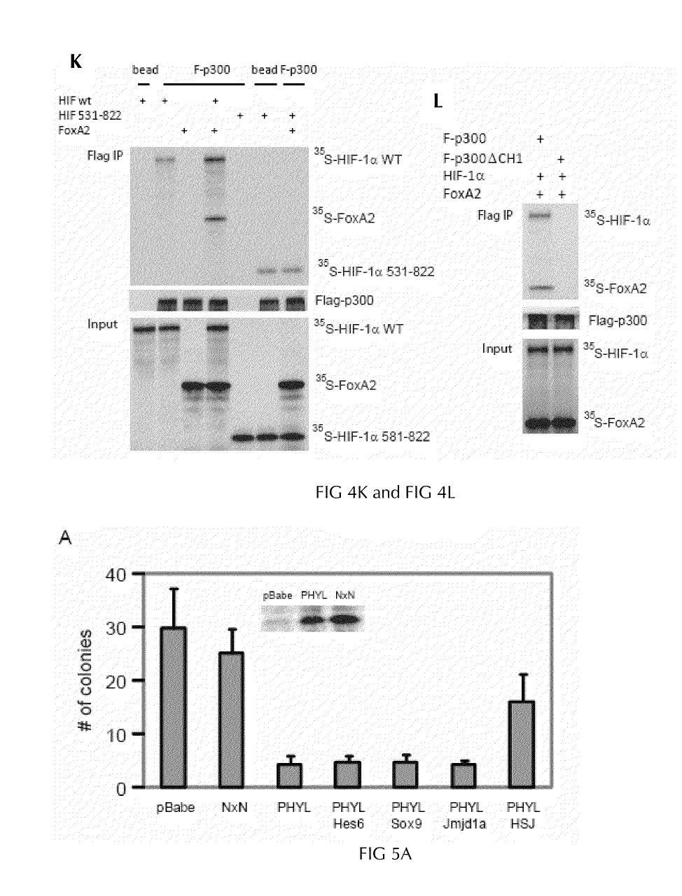

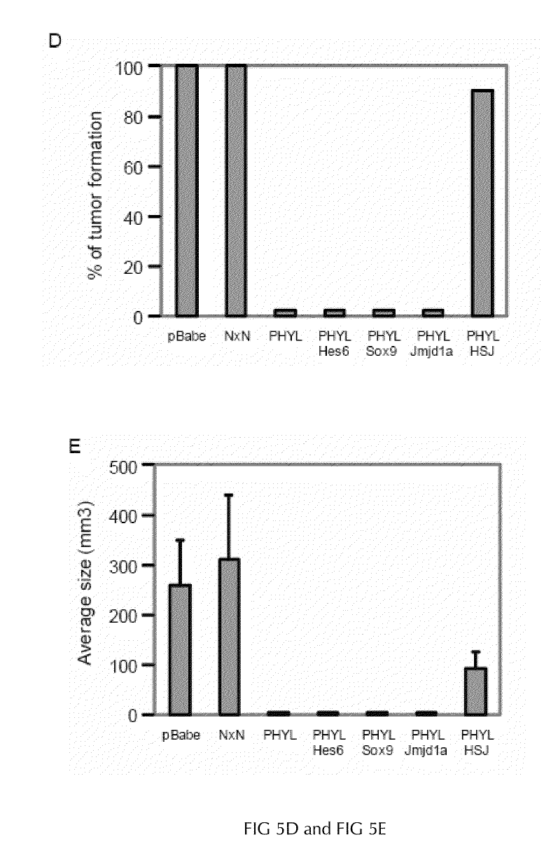

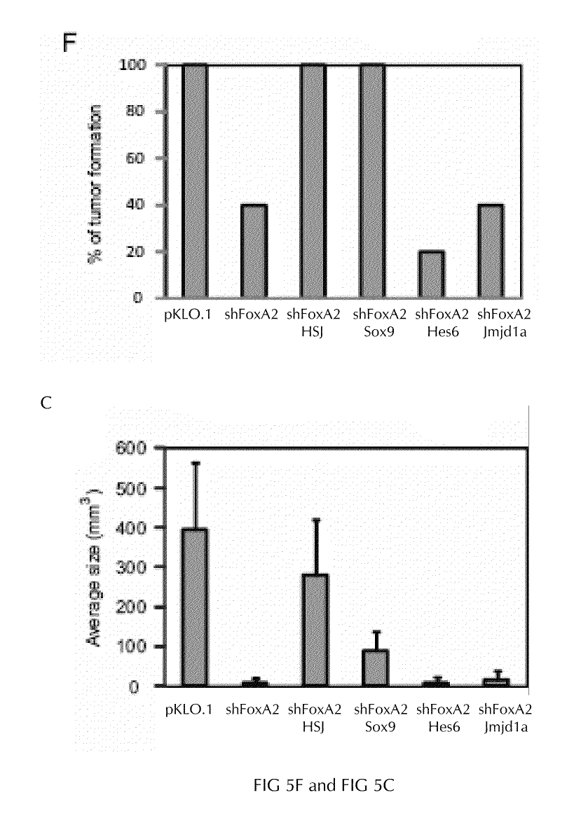

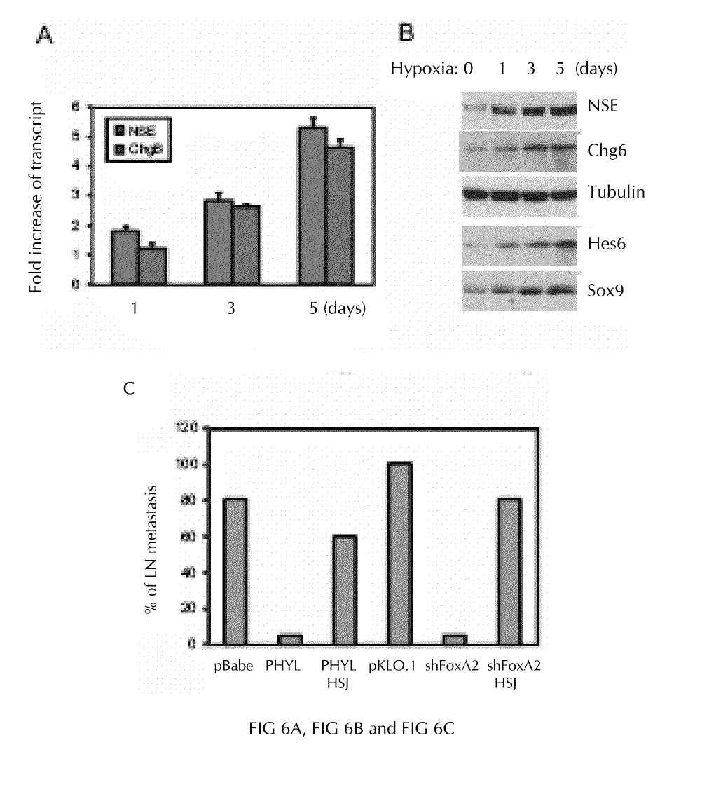

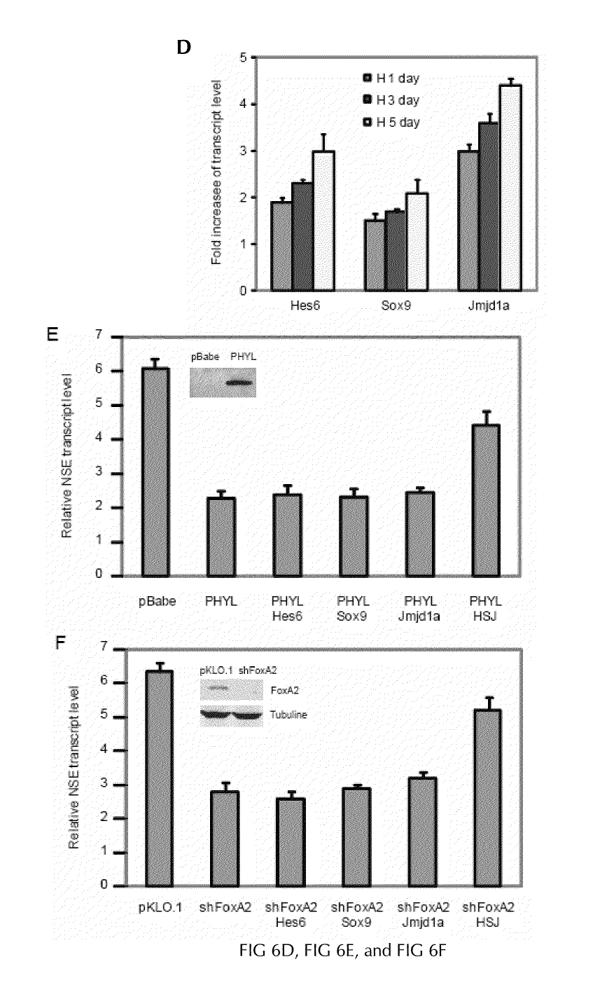

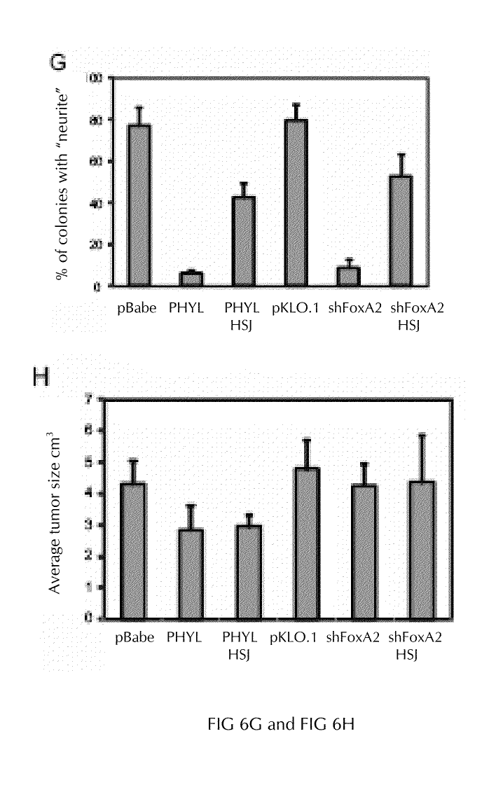

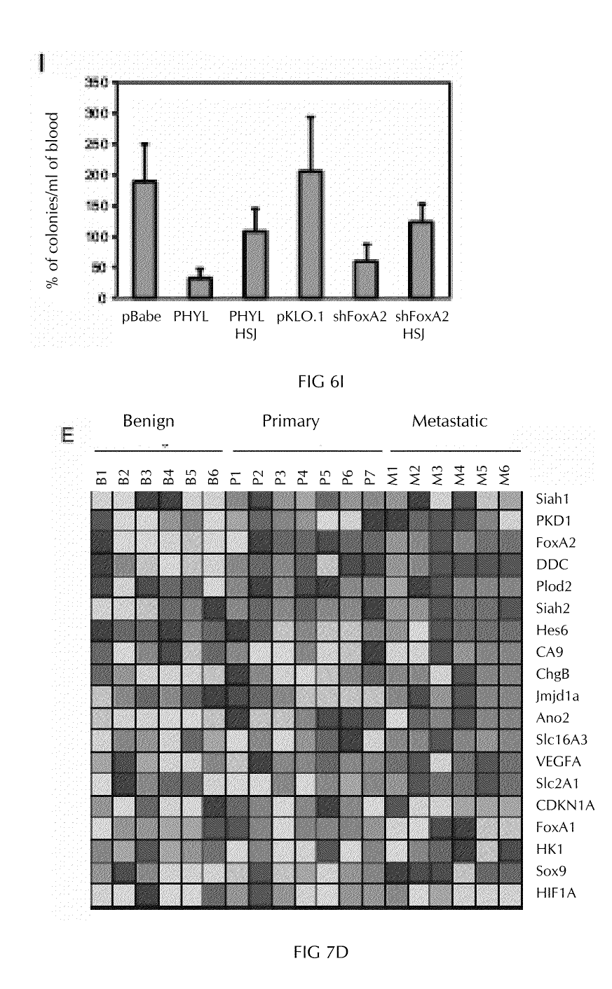

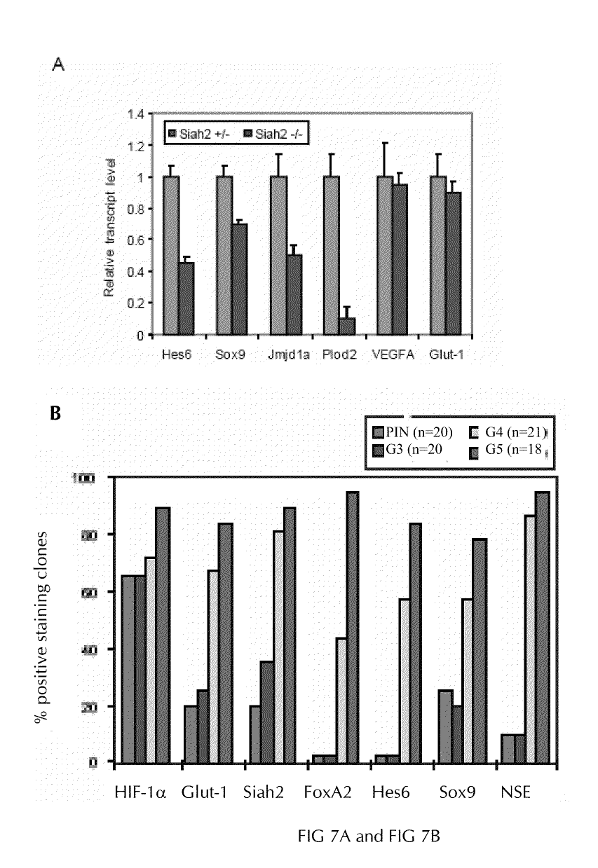

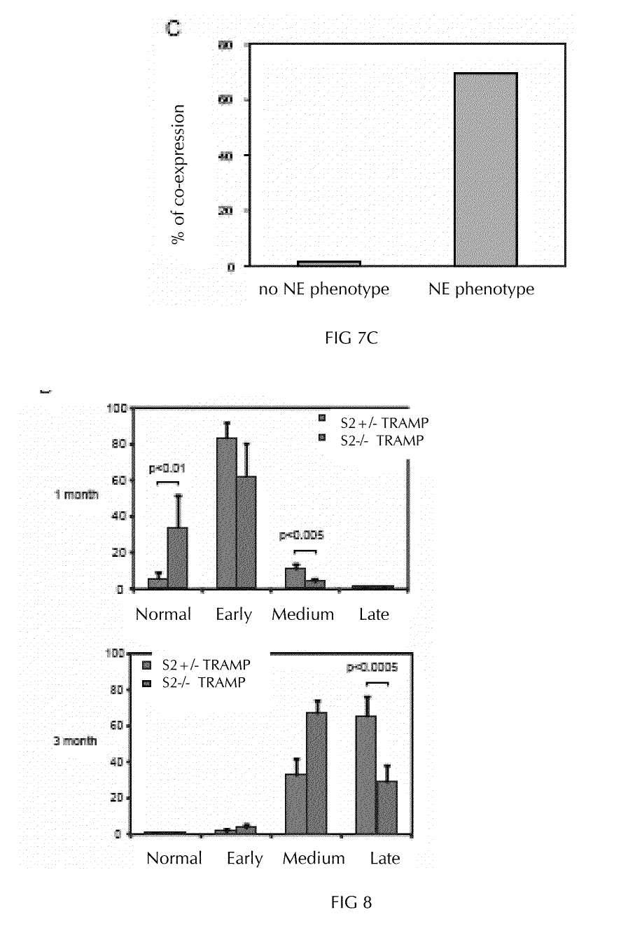

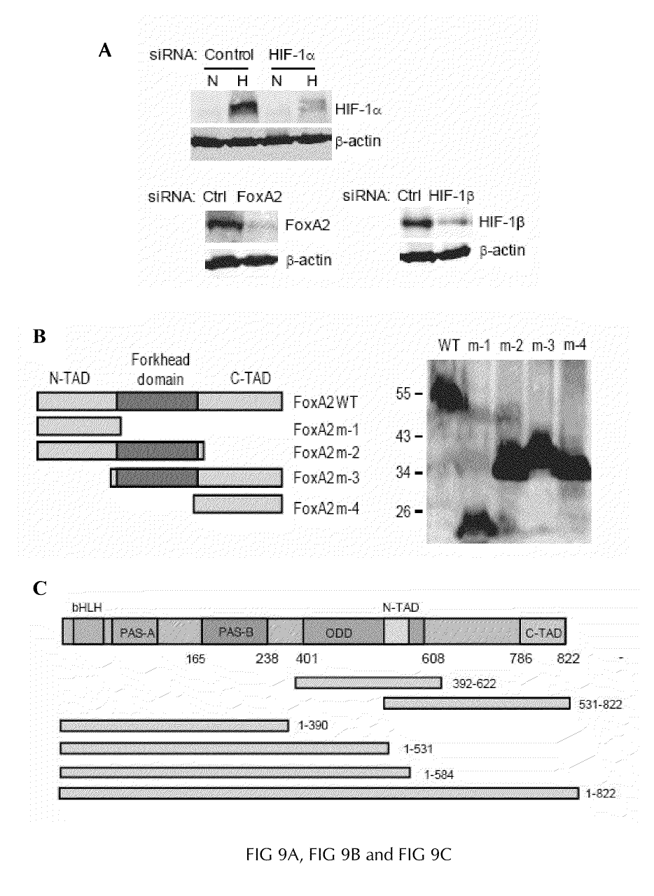

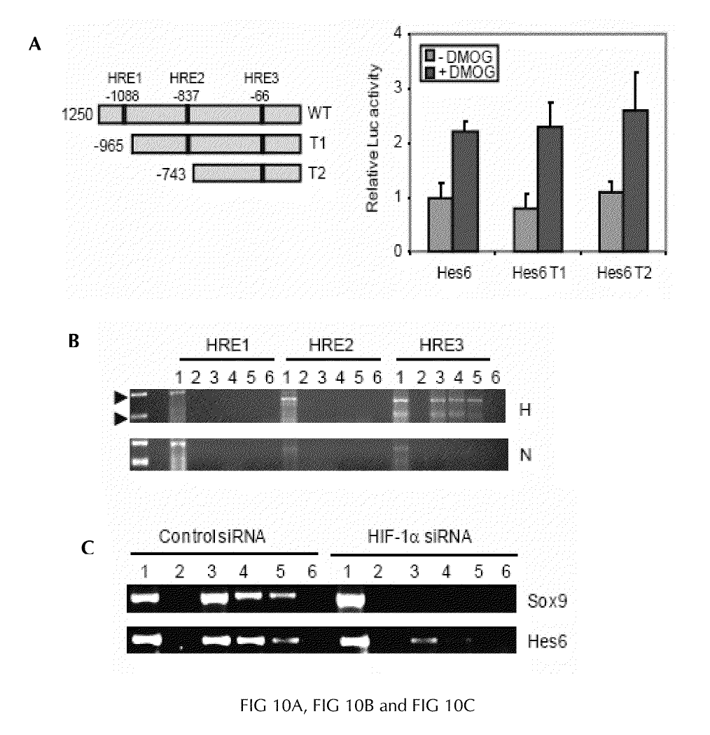

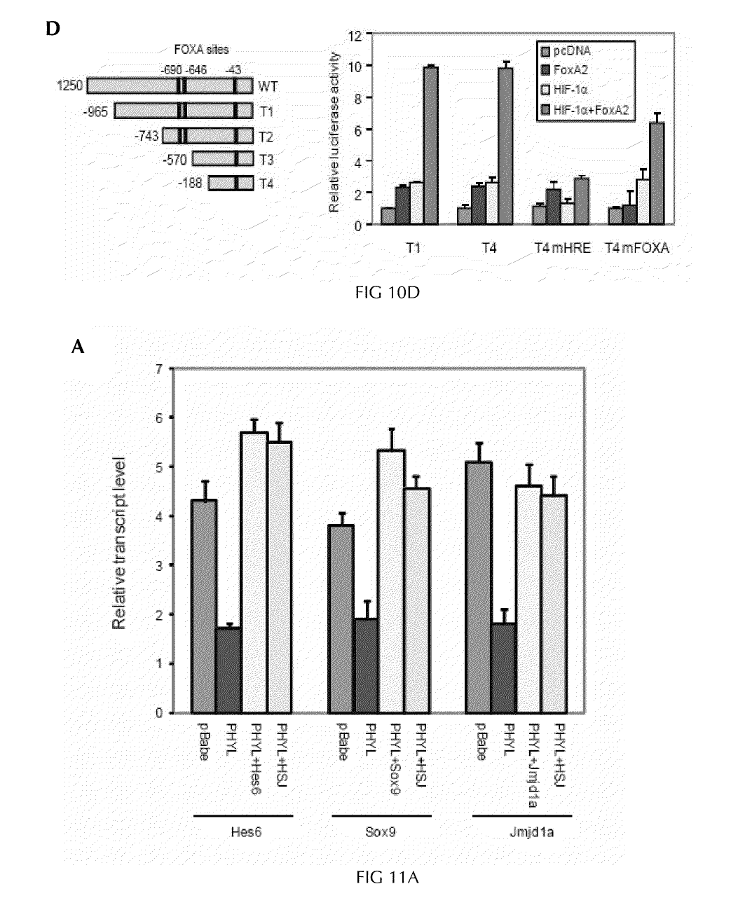

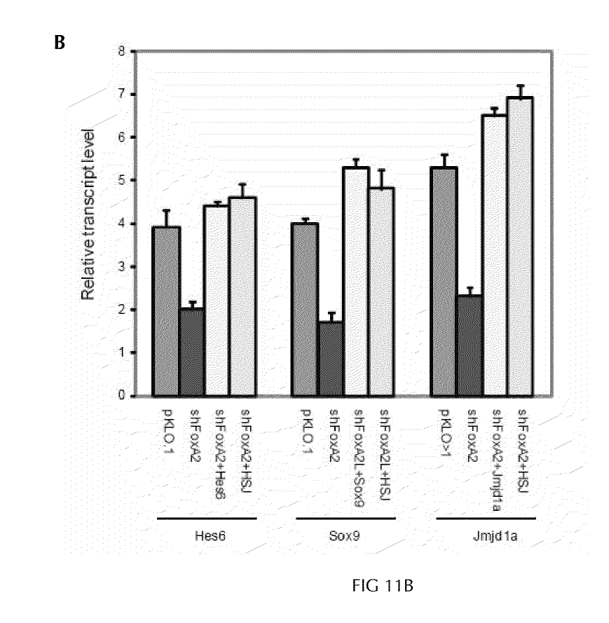

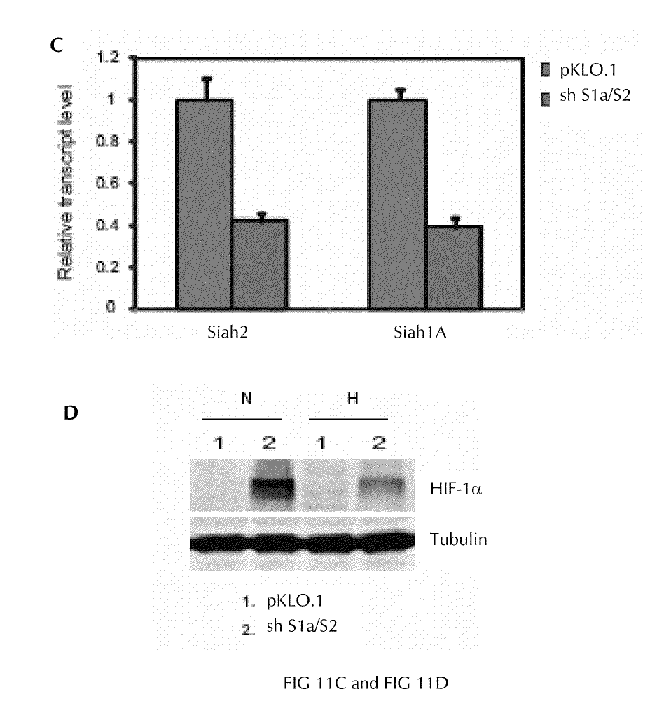

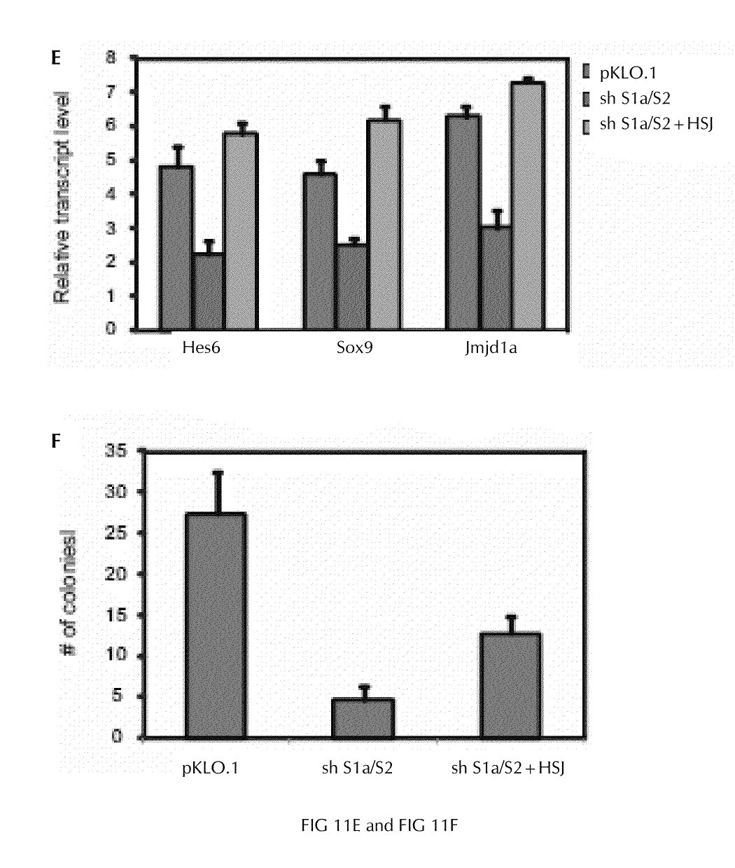

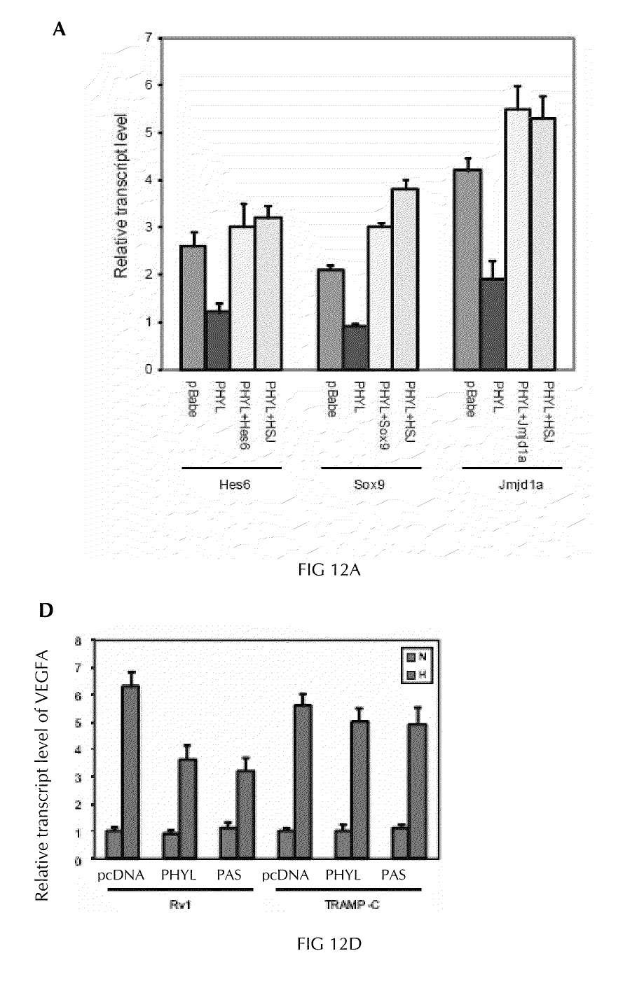

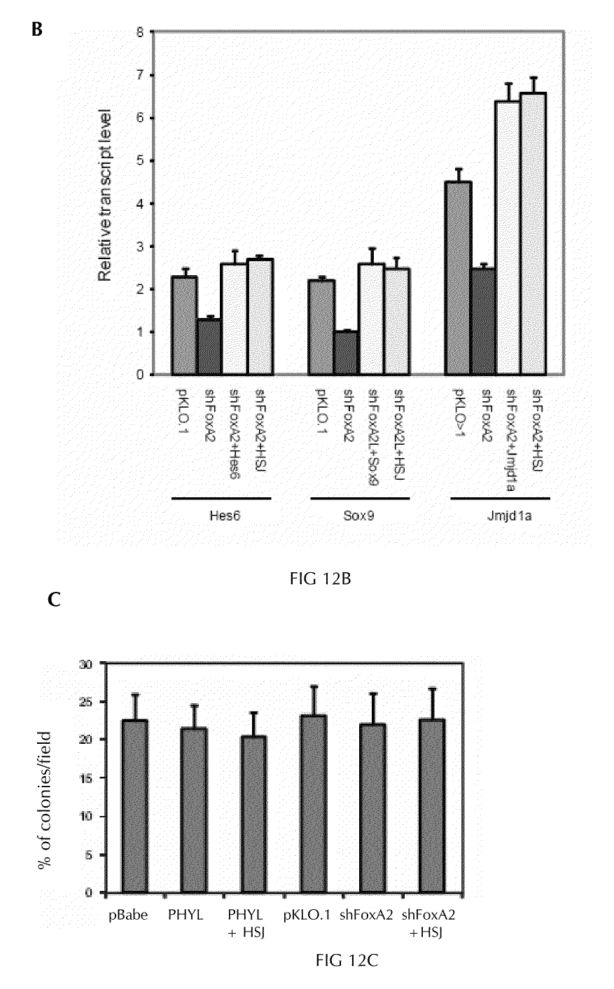

FIGS. 10A-10D (related to FIG. 4) A. Left panel: Diagram of truncation mutants of Hes6 promoter-Luc construct. The 3 potential HREs on the mouse Hes6 gene promoter regions are indicated. Right panel: Identification of a functional HRE in the Hes6 promoter. TRAMP-C cells were transfected with wild type or truncation mutants of the Hes6 promoter-Luc construct. 24-h post transfection, cells were treated with 1 mM of DMOG for 16 h before analysis of luciferase activity. Each column represents mean±SD (standard deviation) for 3 replicates. B. Binding of HIFs and FoxA2 to the functional HRE of Hes6 promoter. TRAMP-C cells were treated with 1% oxygen for 6 h and ChIP assays were performed using antibodies against HIF-1α, HIF-1β and FoxA2. The precipitated materials were used for PCR amplification of regions containing the 3 putative HREs on the Hes6 promoter as well as the Hes6 encoding region as negative control. Lane 1: 10% input; 2: Control rabbit antibody; 3: HIF-1α rabbit antibody; 4: HIF-1β rabbit antibody; 5: FoxA2 goat antibody. 6: control goat IgG. PCR products were subjected to 2% agarose gel electrophoresis. N and H denote normoxia and hypoxia, respectively, and arrows point to the corresponding MW markers (100 and 200 bp). Specific amplification products for HRE1, HRE2, HRE3 and Hes6 encoding region were 200, 160, 149 bp, and 120 bp, respectively. HRE1, 2, 3 refers the potential HRE located at 1088, 837 and 66 by upstream of Hes6 transcriptional start site, respectively (FIG. 10A). C. HIF-1α is required for the binding of FoxA2 to the HRE-containing region of Hes6 and Sox9 promoter. TRAMP-C cells were transfected with HIF-1α siRNAs. 48-h post transfection, cells were treated with 1% oxygen for 5 h. ChIP assays were performed using antibodies against HIF-1α, HIF-1β and FoxA2. The precipitated chromatin was used for PCR amplification of HRE-containing promoter regions of Hes6 or Sox9. PCR products were analyzed by 2% agarose gel. Lane 1: 10% input; 2: Control rabbit antibody; 3: HIF-1α rabbit antibody; 4: HIF-1β rabbit antibody; 5: FoxA2 goat antibody. 6: control goat IgG. D. Left panel: Diagram of truncation mutants of Hes6 promoter-Luc constructs. The 3 potential FOXA sites are indicated (relative position of each FOXA site is noted). The mutant Hes6 promoter sequences were cloned in pGL3b vector upstream of a luciferase reporter. Right panel: The FOXA site on the Hes6 promoter is partially involved in the HIF/FoxA2 synergy. TRAMP-C cells were transfected with various Hes6 promoter-Luc constructs. T4 mHRE and T4 mFOXA refer to mutation of HRE at -66 and mutation of FOXA site at -43, respectively. 24-h post transfection, cells were collected to measure luciferase activity. Each column represents mean±SD for 3 replicates. The first bar in each group represents pcDNA, the second bar represents FoxA2, the third bar represents HIF-1α, the fourth bar represents HIF-1α+FoxA2.

FIGS. 11A-11F (related to FIG. 5) A and B. qRT-PCR analysis for re-expression of Hes6, Sox9, and JmjdlA individually or in combination (HSJ) in PHYL-(FIG. 11A) or shFoxA2 (FIG. 11B)-expressing TARMP-C cells. TRAMP-C cells were stably transfected with PHYL peptide or FoxA2 shRNA. PHYL- or shFoxA2-expressing TRMAP-C cells were then infected with retroviral constructs encoding Hes6, Sox9 or Jmjd1a either alone or in combination (HSJ). The stable transfectants were treated with 1% O₂ for 5 days before isolation of RNA for qRT-PCR analysis of Hes6, Sox9 and Jmjd1a. C. QPCR showing the shRNA knockdown of Siahl a and Siah2 in TRAMP-C cells. The first bar represents pKLO.1 and the second bar represents shS1a/S2. D. Knockdown of Siah1a and Siah2 in TRAMP-C cells reduces HIF-1α level under hypoxia. Cells were treated with 1% O₂ for 6 h and subjected to western blot analysis of HIF-1α. E. TRAMP-C cells were stably transfected with control pKLO.1 vector or FoxA2 shRNA. shSiah1a/2-expressing TRMAP-C cells were then infected with retroviral constructs encoding Hes6, Sox9 or Jmjdl a either alone or in combination (HSJ). The stable transfectants were treated with 1% O2 for 5 days before qRT-PCR analysis of Hes6, Sox9 and Jmjdl a. The first bar represents pKLO.1, the second bar represents shS1a/S2 and the third bar represents shS1a/S2+HSJ. F. Co-expression of Hes6, Sox9 and Jmjd1a partially rescues the colony formation of TRMAP-C cells upon knockdown of Siah2 and Siahl a. Cells were monitored for their ability to form colonies on soft agar for 3 weeks under 1% O2. The number of colonies was scored. In panel A, B, C, E and F, each column represents mean±SD for 3 replicates.

FIGS. 12A-12D (related to FIG. 6) A. qRT-PCR analysis for the re-expression of Hes6, Sox9, and Jmjd1A in PHYL-expressing Rv1 cells. Rv1 cells were stably transfected with control pBabe vector or PHYL. PHYL-expressing Rv1 cells were infected with indicated viral constructs either individually or in combination (HSJ). Stable transfectants were grown in 1% O₂ for 5 days before qRT-PCR analysis. B. qRT-PCR analysis of the re-expression of Hes6, Sox9, and Jmjd1a in shFoxA2-expressing Rv1 cells. Rv1 cells were transfected with control pKLO.l vector or FoxA2 shRNA and were further infected and processed as indicated in panel A. C. PHYL, shFoxA2 or HIF/FoxA2 regulated genes do not affect Rv1 growth on soft agar. Cells were grown on soft agar for 3 weeks at 1% O₂, The number of colonies were scored. Each column in panels A, B, D, E, represents mean±SD of 3 replicates. D. PHYL or IPAS reduced VEGFA transcript in Rv1 cells but not in the TRAMP-C cells. Cells were transfected and grown in 1% O₂ for 6 h before qRT-PCR analysis. The first bar in each group represents N (normal) and the second bar represents H (hypoxia).

### DETAILED DESCRIPTION OF THE INVENTION

The disclosed method and compositions may be understood more readily by reference to the following detailed description of particular embodiments and the Example included therein and to the Figures and their previous and following description.

Disclosed are methods, compounds, and compositions for detecting, diagnosing, determining the prognosis, monitoring, monitoring treatment, and selecting treatment of cancer and metastasis. The disclosed methods, compounds, and compositions are particularly suited for neuroendocrine differentiation (NED)-associated cancers and cancers that could develop neuroendocrine phenotype. It has been discovered that expression of FoxA2 and HIF-1α in cancer cells indicates the presence of or a risk of metastasis of the cancer, the presence of or a risk of neuroendocrine differentiation (NED)-associated cancer. Such forms of cancer and the risk of such forms of cancer indicate a poorer prognosis than cancers that do not exhibit such expression.

The expression of FoxA2 and/or HIF-1α can be expressed or described by any suitable metric. For example, the expression of FoxA2 and/or HIF-1α can be expressed or described by the number of cells expressing FoxA2 and/or HIF-1α, the fraction of cells expressing FoxA2 and/or HIF-1α, and/or the level of FoxA2, the level of HIF-1α, or a combination. The correlation of the expression of FoxA2 and HIF-1α to metastasis and to NED-associated cancer can be used in a variety of ways. For example, the correlation of the expression of FoxA2 and HIF-1α to metastasis and to NED-associated cancer can be used for detecting the presence of or a risk of metastasis of cancer in a subject, treating a subject at risk of metastasis of cancer, identifying an inhibitor of HIF 1 a:FoxA2 function or complex formation, detecting the presence of or a risk of neuroendocrine differentiation (NED)-associated cancer, determining a prognosis of a cancer, determining a treatment for a cancer, monitoring or determining the effect of treatment of a NED-associated cancer, treating NED-associated cancer, identifying an inhibitor of HIF1α:FoxA2 complex formation, detecting neuroendocrine differentiation (NED)-associated cancer, monitoring the risk of metastasis of cancer in a subject, and treating NED-associated cancer.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. HIF-1α

Hypoxia inducible factor-1 (HIF-1) plays a central role in the development and progression of tumors. HIF-1 controls the expression of more than 40 target genes whose protein products play crucial roles in allowing the survival of cells under adverse environmental conditions and in response to radiation or chemotherapy. These include the gene encoding VEGF, which is required for tumor angiogenesis, insulin-like growth factor 2 (IGF2), which promotes tumor cell survival, and glucose transporters 1 and 3, and glycolytic enzymes such as aldolase A and C, hexokinase 1 and 3, lactate dehydrogenase A and PGK.

HIF-1α is a subunit of HIF-1. HIF-1α protein is found in a wide variety of human primary tumors, including neuroendocrine tumors, but only at very low levels in normal tissue. The importance of HIF-1α to cancer is demonstrated by the high incidence of tumors such as renal cell carcinoma, pheochromocytoma and hemingioblastoma of the central nervous system in individuals with loss of function of both alleles of the VHL gene leading to elevated HIF-1α levels. In addition, most cases of sporadic renal cell carcinoma are associated with an early loss of function of the VHL gene and increased HIF-1α levels. Reintroduction of the intact VHL gene into cells derived from renal carcinomas restores HIF-1α to normoxic levels and decreases tumorigenicity. HIF-1α levels are also increased in cancer cells with mutant or deleted PTEN.

Important in the control of HIF abundance is their hydroxylation on proline residue(s) by prolyl hydroxylases (PHD), which is required for their association with and degradation by the pVHL-Elongin B, C ubiquitin ligase complex (Ivan et al. Science 292:464-468, 2001; Jaakkola et al. Science 292:468-472, 2001). At low oxygen concentrations, PHDs, usually exhibit between 50% and 10% of their hydroxylase activity toward their substrates (Stiehl D P, et al.(2006) J Biol Chem 281:23482-23491; Tuckerman J R, et al.(2004) FEBS Lett 576:145-150; Wirthner R, et al.(2007) Methods Enzymol 435:43-60). Consistent with these findings, active degradation of PHD1/3 by Siah2 increases HIF-1α stability under hypoxia (Nakayama K, et al.(2004) Cell 117:941-952). The tight regulation of PHD activity and stability under hypoxic conditions contributes to the availability of HIF-1α protein, enabling transactivation of its target genes, among which VEGF, c-met, CXCR4, and lysyl oxidase have been implicated in tumor growth and metastasis (Erler JT, et al.(2006) Nature 440:1222-1226; Pennacchietti S, et al.(2003) Cancer Cell 3:347-361; Semenza G L (2003) Nat Rev Cancer 3:721-732; Staller P, et al.(2003) Nature 425:307-311).

Many human tumors have been shown to overexpress HIF-1α protein as a result of intratumoral hypoxia and genetic alterations affecting key oncogenes and tumor suppressor genes. In addition, over-expression of HIF-1α correlates with treatment failure and mortality. However, loss of HIF-1 activity has dramatic negative effects on tumor growth, vascularization and energy metabolism in xenograft assays. Therefore inhibition of HIF-1, particularly HIF-1α, represents a promising new approach to cancer therapy since its inhibition may lead to the selective killing of tumor cells over normal cells.

### B. FoxA2

Fox (Forkhead box) proteins are a family of transcription factors that play important roles in regulating the expression of genes involved in cell growth, proliferation, differentiation, and longevity. FoxA2 is expressed in mouse prostate neuroendocrine carcinomas (Chiaverotti et al. 2008, Mirosevich et al. 2006).

The Fox proteins have been further sub-grouped, examples of which are FoxA1, FoxA2, and FoxA3 which were formally known as hepatocyte nuclear factor 3 (HNF3α, HNF3β, and HNF3γ respectively) (Lai et al. Genes Dev 5:416-427, 1991; Lai et al. Genes Dev 4:1427-1436, 1990). They were discovered as controlling liver-specific gene expression. Although the three genes are on different chromosomes (Kaestner et al. Genomics 20:377-385, 1994), they share about 85% sequence identity in the DNA-binding domain (Lai et al. Genes Dev 5:416-427, 1991), which exhibits 82% similarity with the Drosophila forkhead protein (Weigel et al. Cell 63:455-456,1990). The FoxA proteins bind to the same consensus binding site, but each exhibit different affinity. The forkhead domain displays a novel protein folding called “winged helix,” which contains three α-helices flanked by two loops, or “wings” (Clark et al. Nature 364:412-420, 1993). Helix 3 makes primary contacts with the DNA major groove, and wing 2 makes contacts with the minor groove (Clark et al. Nature 364:412-420, 1993).

FoxA2 contains two transactivation domains, N-terminal and C-terminal transactivation domains (N-TAD and C-TAD, respectively), and intrinsic chromatin remodeling activity in the C-terminus (Cirillo et al. 2002).

### C. Cancer

The disclosed methods, compounds, and compositions relate to the diagnosis, prognosis, assessment, monitoring, treatment, etc. of cancer. The disclosed methods, compounds, and compositions can be used in the context of any cancer. However, they are particularly relevant to cancers that can or do exhibit neuroendocrine phenotype and/or neuroendocrine differentiation. For example, the disclosed methods, compounds, and compositions are particularly useful in the context of prostate cancer, lung cancer, pancreatic cancer, or merkel cell carcinoma. Some forms of the disclosed methods involve cancer samples. Thus, useful cancer samples can include, for example, prostate cancer samples, lung cancer samples, pancreatic cancer samples, or merkel cell carcinoma samples. Furthermore, the subject can have prostate cancer, lung cancer, pancreatic cancer, or merkel cell carcinoma.

1. Prostate Cancer

Prostate cancer is most often discovered by physical examination or by screening blood tests, such as the PSA (prostate specific antigen) test. There is some current concern about the accuracy of the PSA test and its usefulness. Suspected prostate cancer is typically confirmed by removing a piece of the prostate (biopsy) and examining it under a microscope. Further tests, such as X-rays and bone scans, can be performed to determine whether prostate cancer has spread.

Prostate cancer can be treated with surgery, radiation therapy, hormonal therapy, occasionally chemotherapy, proton therapy, or some combination of these. The age and underlying health of the man as well as the extent of spread, appearance under the microscope, and response of the cancer to initial treatment are important in determining the outcome of the disease. Since prostate cancer is a disease of older men, many will die of other causes before a slowly advancing prostate cancer can spread or cause symptoms. This makes treatment selection difficult. The decision whether or not to treat localized prostate cancer (a tumor that is contained within the prostate) with curative intent is a patient trade-off between the expected beneficial and harmful effects in terms of patient survival and quality of life. The disclosed methods can be used to guide the selection of the appropriate therapy.

The only test which can fully confirm the diagnosis of prostate cancer is a biopsy, the removal of small pieces of the prostate for microscopic examination. However, prior to a biopsy, several other tools may be used to gather more information about the prostate and the urinary tract. Cystoscopy shows the urinary tract from inside the bladder, using a thin, flexible camera tube inserted down the urethra. Transrectal ultrasonography creates a picture of the prostate using sound waves from a probe in the rectum.

If cancer is suspected, a biopsy is offered. During a biopsy, a urologist obtains tissue samples from the prostate via the rectum. The tissue samples are then examined under a microscope to determine whether cancer cells are present, and to evaluate the microscopic features (or Gleason score) of any cancer found. Tissue samples can be stained for the presence of PSA and other tumor markers in order to determine the origin of malignant cells that have metastasized.

An important part of evaluating prostate cancer is determining the stage, or how far the cancer has spread. Knowing the stage helps define prognosis and is useful when selecting therapies. The most common system is the four-stage TNM system (abbreviated from Tumor/Nodes/Metastases). Its components include the size of the tumor, the number of involved lymph nodes, and the presence of any other metastases.

The most important distinction made by any staging system is whether or not the cancer is still confined to the prostate. In the TNM system, clinical T1 and T2 cancers are found only in the prostate, while T3 and T4 cancers have spread elsewhere. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans should reveal osteoblastic appearance due to increased bone density in the areas of bone metastasis - opposite to what is found in many other cancers that metastasize.

After a prostate biopsy, a pathologist identifies the grade of the tumor. The Gleason system can be used to grade prostate tumors from 2 to 10, where a Gleason score of 10 indicates the most abnormalities. The pathologist assigns a number from 1 to 5 for the most common pattern observed under the microscope, then does the same for the second most common pattern. The sum of these two numbers is the Gleason score. The Whitmore-Jewett stage is another method sometimes used. Proper grading of the tumor is critical, since the grade of the tumor is one of the major factors used to determine the treatment recommendation.

Treatment for prostate cancer can involve watchful waiting, surgery, radiation therapy, High Intensity Focused Ultrasound (HIFU), chemotherapy, cryosurgery, hormonal therapy, or some combination. Which option is generally decided based on the stage of the disease, the Gleason score, and the PSA level. The disclosed methods can be used to further discriminate between therapeutic options based on expression of HIF-1α and FoxA2. Expression of HIF-1α and FoxA2 can indicate the need for a neuroendocrine disease-associated treatment such as chemotherapy, cryoablation, drug therapy, nutritional modification, radiofrequency ablation or surgery.

If the cancer has spread beyond the prostate, treatment options significantly change, so most doctors who treat prostate cancer use a variety of nomograms to predict the probability of spread. Treatment by watchful waiting, HIFU, radiation therapy, cryosurgery, and surgery are generally offered to men whose cancer remains within the prostate. Hormonal therapy and chemotherapy are often reserved for disease which has spread beyond the prostate. However, there are exceptions: radiation therapy may be used for some advanced tumors, and hormonal therapy is used for some early stage tumors. Cryotherapy, hormonal therapy, and chemotherapy can also be offered if initial treatment fails and the cancer progresses.

Watchful waiting, also called “active surveillance,” refers to observation and regular monitoring without invasive treatment. Watchful waiting is often used when an early stage, slow-growing prostate cancer is found in an older man. Watchful waiting may also be suggested when the risks of surgery, radiation therapy, or hormonal therapy outweigh the possible benefits. Other treatments can be started if symptoms develop, or if there are signs that the cancer growth is accelerating (e.g., rapidly rising PSA, increase in Gleason score on repeat biopsy, or expression of HIF-1α and FoxA2 as disclosed herein).

Surgical removal of the prostate, or prostatectomy, is a common treatment either for early stage prostate cancer, or for cancer which has failed to respond to radiation therapy. The most common type is radical retropubic prostatectomy, when the surgeon removes the prostate through an abdominal incision. Another type is radical perineal prostatectomy, when the surgeon removes the prostate through an incision in the perineum, the skin between the scrotum and anus. Radical prostatectomy can also be performed laparoscopically, through a series of small (1 cm) incisions in the abdomen, with or without the assistance of a surgical robot.

Radiation therapy (radiotherapy), can be used to treat all stages of prostate cancer, or when surgery fails. Radiotherapy uses ionizing radiation to kill prostate cancer cells. When absorbed in tissue, Ionizing radiation such as Gamma and x-rays damage the DNA in cells, which increases the probability of apoptosis (cell death). Two different kinds of radiation therapy are used in prostate cancer treatment: external beam radiation therapy and brachytherapy.

External beam radiation therapy uses a linear accelerator to produce high-energy x-rays which are directed in a beam towards the prostate. A technique called Intensity Modulated Radiation Therapy (IMRT) can be used to adjust the radiation beam to conform with the shape of the tumor, allowing higher doses to be given to the prostate and seminal vesicles with less damage to the bladder and rectum. External beam radiation therapy is generally given over several weeks, with daily visits to a radiation therapy center. New types of radiation therapy can have fewer side effects then traditional treatment, one of these is Tomotherapy.

Permanent implant brachytherapy is a popular treatment choice for patients with low to intermediate risk features, can be performed on an outpatient basis, and is associated with good 10-year outcomes with relatively low morbidity. It involves the placement of about 100 small “seeds” containing radioactive material with a needle through the skin of the perineum directly into the tumor while under spinal or general anesthetic. These seeds emit lower-energy X-rays which are only able to travel a short distance. Although the seeds eventually become inert, they remain in the prostate permanently. The risk of exposure to others from men with implanted seeds is generally accepted to be insignificant.

Cryosurgery is another method of treating prostate cancer. It is less invasive than radical prostatectomy, and general anesthesia is less commonly used. Under ultrasound guidance, metal rods are inserted through the skin of the perineum into the prostate. Highly purified Argon gas is used to cool the rods, freezing the surrounding tissue at −196° C. (-320° F.). As the water within the prostate cells freeze, the cells die. The urethra is protected from freezing by a catheter filled with warm liquid.

Hormonal therapy uses medications or surgery to block prostate cancer cells from getting dihydrotestosterone (DHT), a hormone produced in the prostate and required for the growth and spread of most prostate cancer cells. Blocking DHT often causes prostate cancer to stop growing and even shrink. However, hormonal therapy rarely cures prostate cancer because cancers which initially respond to hormonal therapy typically become resistant after one to two years. Hormonal therapy is therefore usually used when cancer has spread from the prostate. It can also be given to certain men undergoing radiation therapy or surgery to help prevent return of their cancer.

Hormonal therapy for prostate cancer targets the pathways the body uses to produce DHT. A feedback loop involving the testicles, the hypothalamus, and the pituitary, adrenal, and prostate glands controls the blood levels of DHT. First, low blood levels of DHT stimulate the hypothalamus to produce gonadotropin releasing hormone (GnRH). GnRH then stimulates the pituitary gland to produce luteinizing hormone (LH), and LH stimulates the testicles to produce testosterone. Finally, testosterone from the testicles and dehydroepiandrosterone from the adrenal glands stimulate the prostate to produce more DHT. Hormonal therapy can decrease levels of DHT by interrupting this pathway at any point.

There are several forms of hormonal therapy. Orchiectomy is surgery to remove the testicles. Because the testicles make most of the body's testosterone, after orchiectomy testosterone levels drop. Now the prostate not only lacks the testosterone stimulus to produce DHT, but also it does not have enough testosterone to transform into DHT.

Antiandrogens are medications such as flutamide, bicalutamide, nilutamide, and cyproterone acetate which directly block the actions of testosterone and DHT within prostate cancer cells. Medications which block the production of adrenal androgens such as DHEA include ketoconazole and aminoglutethimide. Because the adrenal glands only make about 5% of the body's androgens, these medications are generally used only in combination with other methods that can block the 95% of androgens made by the testicles. These combined methods are called total androgen blockade (TAB). TAB can also be achieved using antiandrogens.

GnRH action can be interrupted in one of two ways. GnRH antagonists suppress the production of LH directly, while GnRH agonists suppress LH through the process of downregulation after an initial stimulation effect. Abarelix is an example of a GnRH antagonist, while the GnRH agonists include leuprolide, goserelin, triptorelin, and buserelin. Initially, GnRH agonists increase the production of LH. However, because the constant supply of the medication does not match the body's natural production rhythm, production of both LH and GnRH decreases after a few weeks.

HIFU for prostate cancer utilizes high intensity focused ultrasound (HIFU) to ablate/destroy the tissue of the prostate. During the HIFU procedure, sound waves are used to heat the prostate tissue thus destroying the cancerous cells. Essentially, ultrasonic waves are precisely focused on specific areas of the prostate to eliminate the prostate cancer with minimal risks of affecting other tissue or organs. Temperatures at the focal point of the sound waves can exceed 100° C. HIFU procedure for prostate cancer is performed using a transrectal probe.

2. Lung Cancer

The vast majority of primary lung cancers are carcinomas of the lung, derived from epithelial cells. Lung cancer, the most common cause of cancer-related death in men and the second most common in women, is responsible for 1.3 million deaths worldwide annually. The most common symptoms are shortness of breath, coughing (including coughing up blood), and weight loss.

The main types of lung cancer are small cell lung carcinoma and non-small cell lung carcinoma. This distinction is important because the treatment varies; non-small cell lung carcinoma (NSCLC) is sometimes treated with surgery, while small cell lung carcinoma (SCLC) usually responds better to chemotherapy and radiation. The most common cause of lung cancer is long term exposure to tobacco smoke. The occurrence of lung cancer in non-smokers, who account for fewer than 10% of cases, appears to be due to a combination of genetic factors, radon gas, asbestos, and air pollution, including second-hand smoke.

Lung cancer can be seen on chest x-ray and computed tomography (CT scan). The diagnosis is confirmed with a biopsy. This is usually performed via bronchoscopy or CT-guided biopsy. Treatment and prognosis depend upon the histological type of cancer, the stage (degree of spread), and the patient's performance status. Possible treatments include surgery, chemotherapy, and radiotherapy. With treatment, the five-year survival rate is 14%.

The vast majority of lung cancers are carcinomas-malignancies that arise from epithelial cells. There are two main types of lung carcinoma, categorized by the size and appearance of the malignant cells seen by a histopathologist under a microscope: non-small cell (80.4%) and small-cell (16.8%) lung carcinoma. This classification, based on histological criteria, has important implications for clinical management and prognosis of the disease.

The non-small cell lung carcinomas are grouped together because their prognosis and management are similar. There are three main sub-types: squamous cell lung carcinoma, adenocarcinoma and large cell lung carcinoma. Accounting for 31.1% of lung cancers, squamous cell lung carcinoma usually starts near a central bronchus. Cavitation and necrosis within the center of the cancer is a common finding. Well-differentiated squamous cell lung cancers often grow more slowly than other cancer types. Adenocarcinoma accounts for 29.4% of lung cancers. It usually originates in peripheral lung tissue. Most cases of adenocarcinoma are associated with smoking However, among people who have never smoked (“never-smokers”), adenocarcinoma is the most common form of lung cancer. A subtype of adenocarcinoma, the bronchioloalveolar carcinoma, is more common in female never-smokers, and may have different responses to treatment. Accounting for 10.7% of lung cancers, large cell lung carcinoma is a fast-growing form that develops near the surface of the lung. It is often poorly differentiated and tends to metastasize early.

Small cell lung carcinoma (SCLC, also called “oat cell carcinoma”) is less common. It tends to arise in the larger airways (primary and secondary bronchi) and grows rapidly, becoming quite large. The “oat” cell contains dense neurosecretory granules (vesicles containing neuroendocrine hormones) which give this an endocrine/paraneoplastic syndrome association. While initially more sensitive to chemotherapy, it ultimately carries a worse prognosis and is often metastatic at presentation. Small cell lung cancers are divided into

Limited stage and Extensive stage disease. This type of lung cancer is strongly associated with smoking

The lung is a common place for metastasis from tumors in other parts of the body. These cancers are identified by the site of origin, thus a breast cancer metastasis to the lung is still known as breast cancer. They often have a characteristic round appearance on chest x-ray. Primary lung cancers themselves most commonly metastasize to the adrenal glands, liver, brain, and bone.

Lung cancer staging is an assessment of the degree of spread of the cancer from its original source. It is an important factor affecting the prognosis and potential treatment of lung cancer. Non-small cell lung carcinoma is staged from IA (“one A”, best prognosis) to IV (“four”, worst prognosis). Small cell lung carcinoma is classified as limited stage if it is confined to one half of the chest and within the scope of a single radiotherapy field. Otherwise it is extensive stage.

Performing a chest x-ray is the first step if a patient reports symptoms that may be suggestive of lung cancer. This can reveal an obvious mass, widening of the mediastinum (suggestive of spread to lymph nodes there), atelectasis (collapse), consolidation (pneumonia), or pleural effusion. If there are no x-ray findings but the suspicion is high (such as a heavy smoker with blood-stained sputum), bronchoscopy and/or a CT scan can provide the necessary information. Bronchoscopy or CT-guided biopsy is often used to identify the tumor type. Treatment for lung cancer depends on the cancer's specific cell type, how far it has spread, and the patient's performance status. Common treatments include surgery, chemotherapy, and radiation therapy. If investigations confirm lung cancer, CT scan and often positron emission tomography (PET) can be used to determine whether the disease is localized and amenable to surgery or whether it has spread to the point where it cannot be cured surgically. Blood tests and spirometry (lung function testing) can be used to assess whether the patient is well enough to be operated on. If spirometry reveals poor respiratory reserve (often due to chronic obstructive pulmonary disease), surgery may be contraindicated.

Surgery itself has an operative death rate of about 4.4%, depending on the patient's lung function and other risk factors. Surgery is usually only an option in non-small cell lung carcinoma limited to one lung, up to stage IIIA. This can be assessed with medical imaging (computed tomography, positron emission tomography). A sufficient pre-operative respiratory reserve must be present to allow adequate lung function after the tissue is removed.

Procedures include wedge resection (removal of part of a lobe), segmentectomy (removal of an anatomic division of a particular lobe of the lung), lobectomy (one lobe), bilobectomy (two lobes) or pneumonectomy (whole lung). In patients with adequate respiratory reserve, lobectomy is generally the preferred option, as this minimizes the chance of local recurrence. If the patient does not have enough functional lung for this, wedge resection can be performed. Radioactive iodine brachytherapy at the margins of wedge excision can reduce recurrence to that of lobectomy.

Small cell lung carcinoma can be treated primarily with chemotherapy and radiation. Primary chemotherapy can also be given in metastatic non-small cell lung carcinoma.

The combination regimen depends on the tumor type. Non-small cell lung carcinoma can be treated with cisplatin or carboplatin, in combination with gemcitabine, paclitaxel, docetaxel, etoposide or vinorelbine. In small cell lung carcinoma, cisplatin and etoposide can be used. Combinations with carboplatin, gemcitabine, paclitaxel, vinorelbine, topotecan and irinotecan can also be used.

Adjuvant chemotherapy refers to the use of chemotherapy after surgery to improve the outcome. During surgery, samples are taken from the lymph nodes. If these samples contain cancer, then the patient has stage II or III disease. In this situation, adjuvant chemotherapy can improve survival by up to 15%. For example, the patient can be treated with platinum-based chemotherapy (including either cisplatin or carboplatin).

Radiotherapy is often given together with chemotherapy, and can be used with curative intent in patients with non-small cell lung carcinoma who are not eligible for surgery. This form of high intensity radiotherapy is called radical radiotherapy. A refinement of this technique is continuous hyperfractionated accelerated radiotherapy (CHART), where a high dose of radiotherapy is given in a short time period. For small cell lung carcinoma cases that are potentially curable, in addition to chemotherapy, chest radiation is often recommended.

For both non-small cell lung carcinoma and small cell lung carcinoma patients, smaller doses of radiation to the chest may be used for symptom control (palliative radiotherapy). Unlike other treatments, it is possible to deliver palliative radiotherapy without confirming the histological diagnosis of lung cancer.

Patients with limited stage small cell lung carcinoma are usually given prophylactic cranial irradiation (PCI). This is a type of radiotherapy to the brain, used to reduce the risk of metastasis. More recently, PCI has also been shown to be beneficial in those with extensive small cell lung cancer. In patients whose cancer has improved following a course of chemotherapy, PCI has been shown to reduce the cumulative risk of brain metastases within one year from 40.4% to 14.6%.

Extracranial stereotactic radiation can be used in the treatment of early-stage lung cancer. In this form of radiation therapy, very high doses are delivered in a small number of sessions using stereotactic targeting techniques. Its use is primarily in patients who are not surgical candidates due to medical comorbidities.

Radiofrequency ablation can be used in the treatment of bronchogenic carcinoma. It is done by inserting a small heat probe into the tumor to kill the tumor cells.

Various molecular targeted therapies have been developed for the treatment of advanced lung cancer. Gefitinib (Iressa) is one such drug, which targets the tyrosine kinase domain of the epidermal growth factor receptor (EGF-R) which is expressed in many cases of non-small cell lung carcinoma. Erlotinib (Tarceva), another tyrosine kinase inhibitor, has been shown to increase survival in lung cancer patients and has recently been approved by the FDA for second-line treatment of advanced non-small cell lung carcinoma. The angiogenesis inhibitor bevacizumab (in combination with paclitaxel and carboplatin) can be used to improve the survival of patients with advanced non-small cell lung carcinoma.

Other treatments include cyclo-oxygenase-2 inhibitors, the apoptosis promoter exisulind, proteasome inhibitors, bexarotene, vaccines, ras proto-oncogene inhibition, phosphoinositide 3-kinase inhibition, histone deacetylase inhibition, and tumor suppressor gene replacement.

3. Merkel Cell Cancer

Merkel cell cancer, also called neuroendocrine carcinoma of the skin or trabular cancer, is a highly aggressive, rare cancer. Merkel cell carcinoma is a very rare disease in which malignant (cancer) cells form in the skin. It arises from hormone-producing cells just beneath the skin and in the hair follicles, and it occurs in the head and neck region. Merkel cd Is are found in the top layer of the skin. These cells are very close to the slag endings that receive the sensation of touch. Merkel cell carcinoma starts most often in areas of skin exposed to the sun, especially the head and neck, as well as the arms, legs, and trunk. Merkel cell carcinoma tends to grow quickly and to metastasize at an early stage. It usually spreads first to nearby lymph nodes and then may spread to lymph nodes or skin in distant parts of the body, lungs, brain, bones, or other organs.

The prognosis and treatment options are dependent on several factors such as the stage of the cancer (based on size and metastasis) and location. How deeply the tumor has grown into the skin can be a factor as well. Like most cancers, early diagnosis and treatment are key to preventing metastasis. Upon diagnosis, most patients are treated surgically to remove the cancerous cells and some surrounding healthy cells. Sometimes the nearby lymph nodes will be removed during surgery as well due to the possibility that they, too, could contain cancer cells.

Surgery is usually not sufficient to control Merkel cell cancer by itself

Radiotherapy is a common part of treatment of Merkel cell cancers. The radiotherapy fields used are usually very large to cover large areas of skin because of Merkel cell cancer's unusual behavior in spreading through the skin and spreading to lymph nodes. Adjuvant radiotherapy has been shown to be effective in reducing recurrence and increasing five year survival of patients with Merkel Cell Carcinoma. Patients who present with no metastases and a negative sentinel lymph node biopsy have a good prognosis when treated with surgery and radiotherapy. These patients have approximately a 90% survival at five years.

Merkel cell cancer that has metastasized may respond to treatment with chemotherapy and/or radiation. Curing the disease is not likely but this therapy can be effective in shrinking the tumor prior to surgery or shrinking the tumor as the first treatment if surgery is not an option. Chemotherapy uses drugs to kill the cancer cells or stop them from dividing. Depending on the type of drug, chemotherapy may be injected, swallowed or applied to your skin.

Sentinel lymph node biopsy detects Merkel Cell cancer spread in one third of patients whose tumors would have otherwise been clinically and radiologically understaged and who may not have received treatment to the involved node bed. There is a significant benefit of adjuvant nodal therapy when the biopsy is positive. Thus, sentinel lymph node biopsy is important for both prognosis and therapy and should be performed routinely for patients with Merkel Cell cancer.

4. Pancreatic Cancer

Pancreatic cancer is the fourth leading cause of cancer death in men and in women and each year ˜28,000 Americans die of the disease (Greenlee, et al., C A Cancer J. Clin. 50:7-33, 2000). The optimal treatment for pancreatic cancer is usually surgery. Many times, pancreatic cancer is not diagnosed early on because there may not be immediate symptoms in the early stages. Because pancreatic cancer is often advanced by the time it is diagnosed, very few pancreatic tumors can be removed by surgery.

Treatment options often depend on whether or not metastasis has occurred. When the tumor has not spread out of the pancreas but cannot be removed, radiation therapy and chemotherapy together may be recommended. When the tumor has spread (metastasized) to other organs such as the liver, chemotherapy alone is usually used. The standard chemotherapy drug is gemcitabine, but other drugs may be used. Gemcitabine can help approximately 25% of patients.

The prognosis for patients with pancreatic cancer that can be surgically removed can often times be very good. Chemotherapy and radiation are often given after surgery to increase the cure rate (this is called adjuvant therapy). However, in more than 80% of patients the tumor has already spread and cannot be completely removed at the time of diagnosis. For pancreatic cancer that cannot be removed completely with surgery, or cancer that has spread beyond the pancreas, a cure is not possible and the average survival is usually less than 1 year. Clinical trials may be recommended to these patients. Unfortunately, 95% of the people diagnosed with pancreatic cancer have less than 5 years to live.

5. Neuroendocrine Differentiation-Associated (NED) Cancer

The disclosed methods, compounds, and compositions also relate to the diagnosis, prognosis, assessment, monitoring, treatment, etc. of NED-associated cancer. NED-associated cancer refers to a cancer that exhibits neuroendocrine phenotype, that is a form of neuroendocrine carcinoma, and/or that exhibits neuroendocrine differentiation.

The neuroendocrine system is a combination of the neurocrine system and the endocrine system and thus neuroendocrine cells can receive neuronal input (neurotransmitters released by nerve cells) and, as a consequence of this input, release message molecules (hormones) to the blood. Tumor cells from different tissues can begin to differentiate or transform into a neuroendocrine phenotype resulting in NED-associated tumors or cancer. The neuroendocrine phenotype results from the presence of neuroendocrine secretory granules in the cells.

Provided herein are methods of detecting the presence of or a risk of neuroendocrine differentiation (NED)-associated cancer, the method comprising detecting one or more cells in a sample that express FoxA2 and HIF-1α, wherein detection of cells that express FoxA2 and HIF-1α indicates the presence of or a risk of NED-associated cancer. The detection of cells can be performed with the methods disclosed herein. Also disclosed are methods of detecting neuroendocrine differentiation (NED)-associated cancer, the method comprising detecting one or more cells in a cancer sample that express FoxA2 and HIF-1α, wherein detection of cells that express FoxA2 and HIF-1α indicate NED-associated cancer. FoxA2 and HIF-1α can both be present at or above respective reference levels in the cells that express FoxA2 and HIF-1α, wherein the presence of FoxA2 and HIF-1α at or above the respective reference levels indicate NED-associated cancer.

D. Antibodies

Disclosed herein are antibodies that specifically bind to antigens, immunogens, and epitopes of interest. For example, disclosed are antibodies that specifically bind to HIF-1α or FoxA2. Such antibodies can be used in and with the disclosed methods and compositions. For example, HIF-1α and FoxA2 antibodies can be used to detect HIF-1α and FoxA2, respectively. Of particular interest, such antibodies can be used to detect HIF-1α and FoxA2 in cancer cells. For example, disclosed are polyclonal antibodies specific for HIF-1α raised in a mammal using HIF-1α protein, or an immunogenic fragment thereof as the immunogen.

For example, disclosed are polyclonal antibodies specific for HIF-1α that were, for example, raised in rabbits using the affinity purified recombinant GST-HIF-1α protein as the immunogen. Also disclosed is anti-HIF-1α serum generated, for example, in rabbits using a synthetic peptide corresponding to HIF-1α or fragments thereof. Also disclosed are monoclonal antibodies specific for HIF-1α. Also disclosed are polyclonal antibodies specific for FoxA2 that were, for example, raised in rabbits using the affinity purified recombinant GST-FoxA2 protein as the immunogen. Also disclosed is anti-FoxA2 serum generated, for example, in rabbits using a synthetic peptide corresponding to FoxA2 or fragments thereof. Also disclosed are monoclonal antibodies specific for FoxA2.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with HIF-1α or FoxA2. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding extracellular fragments of HIF-1α or FoxA2 expressed as a fusion protein with human IgG1 or an epitope tag is injected into the host animal according to methods known in the art (e.g., Kilpatrick KE, et al. Hybridoma. 1998 December; 17(6):569-76; Kilpatrick KE et al. Hybridoma. 2000 August; 19(4):297-302, which are incorporated herein by referenced in full for the methods of antibody production) and as described in the examples.

An alternate approach to immunizations with either purified protein or DNA is to use antigen expressed in Baculovirus. The advantages to this system include ease of generation, high levels of expression, and post-translational modifications that are highly similar to those seen in mammalian systems. Use of this system involves expressing the immunogenic fragment of HIF-1α or FoxA2 as fusion proteins with a signal sequence fragment. The antigen is produced by inserting a gene fragment in-frame between the signal sequence and the mature protein domain of HIF-1α or FoxA2 nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against HIF-1α or FoxA2. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones can be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

The term “antibody” as used herein is meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)₂, Fab′, Fab and the like, including hybrid fragments. Thus, fragments of the antibodies that retain the ability to bind their specific antigens are provided. For example, fragments of antibodies which maintain HIF-1α or FoxA2 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or Baculovirus expression system. For example, one can determine the active domain of an antibody from a specific hybridoma that can cause a biological effect associated with the interaction of the antibody with HIF-1α or FoxA2. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller MJ et al. Nucl. Acids Res. 10:6487-500 (1982).

Techniques can also be adapted for the production of single-chain antibodies specific to an antigenic protein of the present disclosure (see e.g., U. S. Pat. No. 4,946,778). In addition, methods can be adapted for the construction of F (ab) expression libraries (see e.g., Huse, et al., 1989 Science 246: 1275-1281) to allow rapid and effective identification of monoclonal F (ab)fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2)fragment produced by pepsin digestion of an antibody molecule; (ii) an Fab fragment generated by reducing the disulfide bridges of an F ((ab′))(2)fragment; (iii) an F (ab)fragment generated by the treatment of the antibody molecule with papain and a reducing agent and (iv) F (v), fragments.

Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding (Bedzyk et al., 1990; Chaudhary et al., 1990). The linker is chosen to permit the heavy chain and light chain to bind together in their proper conformational orientation. See, for example, Huston, J. S., et al., Methods in Enzym. 203:46-121 (1991), which is incorporated herein by reference. These Fvs lack the constant regions (Fc) present in the heavy and light chains of the native antibody.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

The encoded antibodies can be anti-idiotypic antibodies (antibodies that bind other antibodies) as described, for example, in U.S. Pat. No. 4,699,880. Such anti-idiotypic antibodies could bind endogenous or foreign antibodies in a treated individual, thereby to ameliorate or prevent pathological conditions associated with an immune response, e.g., in the context of an autoimmune disease.

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

The antibody can be bound to a substrate or labeled with a detectable moiety or both bound and labeled. The detectable moieties contemplated with the present compositions include fluorescent, enzymatic and radioactive markers.

### E. Inhibitors

Inhibitors can be used to inhibit gene expression or protein function/activity. The inhibition can be complete inhibition or partial inhibition Inhibition can be a result of direct or indirect inhibition meaning the inhibitors acts directly on the target (protein or gene to be inhibited) or the inhibitor can act indirectly via a different protein or gene upstream of the target. An inhibitor can be a small molecule, compound, protein or nucleic acid.

Useful inhibitors for use in the disclosed methods include, for example, HIF-1α inhibitors, FoxA2 inhibitors, Siah2 inhibitors, inhibitors of HIF-1α:FoxA2 complex, inhibitors of HIF-1α:FoxA2 complex function, inhibitors of HIF-1α:FoxA2 complex formation, inhibitors of HIF-1α:FoxA2-regulated genes, and inhibitors of metastasis. “HIF-1α:FoxA2-regulated genes” refers to the subset of HIF-1α-regulated genes that are regulated through cooperation of HIF-1α and FoxA2. Examples include but are not limited to Hes6, Sox9, Jmjd1a and Plod2.

“Inhibit,” “inhibiting,” and “inhibition” and like terms mean to decrease an activity, response, condition, disease, or other biological parameter. This can include but is not limited to the complete ablation of the expression, activity, response, condition, or disease. This may also include, for example, a 10% reduction in the expression, activity, response, condition, or disease as compared to the native or control level. Thus, the reduction can be a 10, 20, 30, 40, 50, 60, 70, 80, 90, 100%, or any amount of reduction in between as compared to native or control levels.

1. HIF-1α Inhibitor

The compositions in the disclosed methods of treatment can comprise an inhibitor of HIF-1α. The compositions can also comprise multiple inhibitors of HIF-1α. For example, one, two or three inhibitors can be used to treat a subject at risk of metastasis of cancer. The inhibitors can be used simultaneously or consecutively. HIF-1α inhibitors would be known by one of skill in the art. ““HIF-1a inhibitor” refers to any molecule or composition that counteracts, reduces, suppresses, inhibits, blocks, or hinders the activity of a HIF-1α molecule or a fragment thereof. The inhibitor can act directly or indirectly. This includes Siah2 inhibitors, which reduce, suppress, inhibit, block, or hinder the activity of HIF-1α.

The inhibitor of HIF-1α can comprise a Siah2 inhibitor. A Siah2 inhibitor is an example of an indirect inhibitor of HIF-1α:FoxA2 regulated genes. Siah2 is a molecule upstream of HIF-1α and therefore Siah2 can inhibit HIF-1α which results in inhibition of one or more HIF-1α:FoxA2 regulated genes. Siah2 inhibitors can comprise a variety of molecules, for example a PHYL peptide. Other Siah2 inhibitors include, but are not limited to, menadione and a Siah RING mutant which acts as a dominant-negative form of the protein. “Siah2 inhibitor” refers to any molecule or composition that counteracts, reduces, suppresses, inhibits, blocks, or hinders the activity of a Siah2 molecule or a fragment thereof. The inhibitor can act directly or indirectly on Siah2.

The HIF-1α inhibitor can comprise a prolyl hydroxylase (PHD) inhibitor. PHDs act upstream of HIF-1α and regulate the oxygen-dependent degradation of HIF-1α. Examples of inhibitors of prolyl hydroxylation include, without limitation, 2-oxoacid molecules and derivatives thereof and 2-oxoglutarate analogs, such as N-oxalyiglyeinc (NOG) or dimethyloxalylglycine (DMOG). For example, see WO2005/094236 and Warnecke et al. (2003 FASEB J. 17, 1186-1188), both of which are incorporated herein by reference. Other HIF-1α inhibitors include, but are not limited to, PX-478, NSC 644221, EZN-2968, FIH-1 and 2-methoxyestradiol.

2. FoxA2 Inhibitor

The compositions in the disclosed methods of treatment can comprise an inhibitor of FoxA2. The inhibitor of FoxA2 can be shRNA. The shRNA will inhibit gene expression of FoxA2 thus preventing the interaction of FoxA2 with HIF-1α. “FoxA2 inhibitor” refers to any molecule or composition that counteracts, reduces, suppresses, inhibits, blocks, or hinders the activity of a FoxA2 molecule or a fragment thereof. The inhibitor can act directly or indirectly on FoxA2.

Compositions for use in the disclosed methods can comprise, for example, a compound or composition that inhibits p300 or reduces p300 recruitment. p300 is a co-activator of HIF transcriptional activity. Inhibitors of FoxA2 can result in inhibition or reduction of p300 recruitment. FoxA2 is involved in the recruitment of p300 to the HIF complex and therefore the inhibition of FoxA2 can result in the inhibition of p300 recruitment. p300 recruitment can refer to, for example, an environment or conditions that allow p300 to move, either on its own or under the influence of other molecule(s), towards a molecule or component of interest (for example, the HIF-1α:FoxA2 complex).

3. HIF-1α:FoxA2 Inhibitor

The compositions in the disclosed methods of treatment can comprise an inhibitor of the HIF-1α:FoxA2 complex. These inhibitors may be the same or different from the disclosed inhibitors of HIF-1α and FoxA2 individually. The HIF-1α:FoxA2 complex comprises a HIF-1α:FoxA2 interaction domain. One useful factor to inhibiting HIF-1α:FoxA2 is the HIF-1α:FoxA2 interaction domain. The HIF-1α:FoxA2 interaction domain is the point of contact between HIF-1α and FoxA2. The interaction domain, in the very least, comprises some if not all of the N-terminal transactivation domain (N-TAD) of FoxA2 and the basic helix-loop-helix—PAS (bHLH-PAS) domain of HIF-1α (amino acids 1-390).

The HIF-1α:FoxA2 inhibitors can also inhibit the function of the HIF-1α:FoxA2 complex. The HIF-1α:FoxA2 complex function comprises any activity of the complex including but not limited to transcription activation of Hes6, Sox9, Jmjdl a or Plod2 or the recruitment of p300 to the complex.

The HIF-1α:FoxA2 inhibitors can also disrupt formation of HIF-1α:FoxA2 complex. The HIF-1α:FoxA2 complex can be disrupted in a variety of ways known to those of skill in the art. For example, the inhibitors can disrupt formation of HIF-1α:FoxA2 by competing for the HIF-1α:FoxA2 interaction domain. Competing for the interaction domain can be accomplished, for example, with peptides containing the sequence of the interaction action. The HIF-1α:FoxA2 inhibitors can also inhibit the formation of a HIF-1α:FoxA2 complex. Inhibiting formation of the HIF-1α:FoxA2 complex can be accomplished, for example, by blocking the interaction domain thereby preventing interaction of HIF-1α and FoxA2 thus inhibiting formation of the HIF-1α:FoxA2 complex. Blocking the interaction domain can be accomplished, for example, with antibodies to the interaction domain which prevent formation of the HIF-1α:FoxA2 complex. There are other ways to block the interaction domain such as with peptides, compounds or nucleic acids that can be used in the disclosed methods. Formation of a HIF-1α:FoxA2 complex can also be inhibited by mutating the HIF-1α and/or FoxA2 protein. A mutation in the HIF-1α:FoxA2 interaction domain can result in the inability to interact and form a complex.

### F. Pharmaceutical Compositions and Carriers

The disclosed methods, such as the disclosed methods for treatment, can use pharmaceutical compositions and carriers. Many such pharmaceutical compositions and carriers are known, as are principles of their formulation and use. The disclosed compositions can be administered in vivo either alone or in a pharmaceutically acceptable carrier. By “pharmaceutically acceptable” is meant a material that is not biologically or otherwise undesirable, i.e., the material can be administered to a subject, along with the composition disclosed herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into microparticles, liposomes, or cells).

1. Pharmaceutically Acceptable Carriers

The compositions disclosed herein can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

2. Nanoparticles, Microparticles, and Microbubbles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, nanoworms, fullerene-like materials, inorganic nanotubes, dendrimers (such as with covalently attached metal chelates), nanofibers, nanohoms, nano-onions, nanorods, nanoropes and quantum dots.

Microspheres (or microbubbles) can also be used with the methods disclosed herein. Microspheres containing chromophores have been utilized in an extensive variety of applications. the monodispersity of the microspheres can be important.

Nanoparticles, such as, for example, metal nanoparticles, metal oxide nanoparticles, or semiconductor nanocrystals can be incorporated into microspheres. The nanoparticle can be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Nanoshells can be formed with a core of a dielectric or inert material such as silicon, coated with a material such as a highly conductive metal which can be excited using radiation such as near infrared light (approximately 800 to 1300 nm). Upon excitation, the nanoshells emit heat. The resulting hyperthermia can kill the surrounding cell(s) or tissue. The combined diameter of the shell and core of the nanoshells ranges from the tens to the hundreds of nanometers. Near infrared light is advantageous for its ability to penetrate tissue. Other types of radiation can also be used, depending on the selection of the nanoparticle coating and targeted cells. Examples include x-rays, magnetic fields, electric fields, and ultrasound.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

3. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

4. Constructs, Vectors and Expression Systems

The disclosed methods, such as the disclosed methods for treatment, can use nucleic acids and expression systems. For example, in some forms, the disclosed methods of treating a subject comprise administering to a subject a composition that inhibits expression of one or more HIF-1α:FoxA2-regulated genes, wherein the composition comprises a vector. Inhibition of expression can be accomplished in a variety of ways. Some ways of inhibiting expression make use of nucleic acids. For example, shRNA, siRNA, antisense RNA, nucleic acids encoding inhibitors or suppressors of expression can be used.

The disclosed compositions can be used with any suitable expression system. Recombinant expression is usefully accomplished using a vector, such as a plasmid. The vector can include a promoter operably linked to nucleic acid encoding an inhibitor of HIF-1α:FoxA2-regulated genes. The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5’ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

i. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes; they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma, I.M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

ii. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIll E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T.F., et al., Mol. Cell Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

iii. Markers

The vectors can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

G. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for identifying compounds that inhibit HIF-1α:FoxA2 complex formation, the kit comprising HIF-1α, FoxA2, antibodies to HIF-1α and/or FoxA2 and reagents to detect binding. The kits also can contain beads or other solid substrates.

I. Mixtures

Disclosed are mixtures formed by performing or preparing to perform the disclosed method. For example, disclosed are mixtures comprising compounds and HIF-1α or FoxA2.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

J. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising cells, compounds, and instruments for detecting binding (i.e. detecting signals from labeled probes).

K. Data Structures and Computer Control

Disclosed are data structures used in, generated by, or generated from, the disclosed method. Data structures generally are any form of data, information, and/or objects collected, organized, stored, and/or embodied in a composition or medium.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods, compounds, and compositions are applicable to numerous areas including, but not limited to, prognosing, diagnosing, monitoring, treating, monitoring treatment of diseases such as cancers, particularly metastatic and neuroendocrine-associated cancer, and cancer with potential for such. Other uses include identification of compounds to inhibit HIF-1α pathway. Other uses are disclosed, apparent from the disclosure, and/or will be understood by those in the art.

### Methods

Disclosed are methods for detection, diagnosis, prognosis, monitoring, treatment, monitoring treatment, and selecting treatment of cancer and metastasis, and for identifying compounds and compositions for such uses. For example, disclosed are methods for detecting the presence of or a risk of metastasis of cancer in a subject, treating a subject at risk of metastasis of cancer, identifying an inhibitor of HIF 1 a:FoxA2 function or complex formation, detecting the presence of or a risk of neuroendocrine differentiation (NED)-associated cancer, determining a prognosis of a cancer, determining a treatment for a cancer, monitoring or determining the effect of treatment of a NED-associated cancer, treating NED-associated cancer, identifying an inhibitor of HIF1α:FoxA2 complex formation, detecting neuroendocrine differentiation (NED)-associated cancer, monitoring the risk of metastasis of cancer in a subject, and treating NED-associated cancer.

For example, disclosed are methods for detecting the presence of or a risk of metastasis of cancer in a subject. Such methods can comprise detecting one or more cells in a cancer sample from the subject that express FoxA2 and HIF-1α, wherein detection of cells that express FoxA2 and HIF-1α indicate the presence of or a risk of metastasis of cancer in the subject. Also disclosed are methods of detecting the presence of or a risk of neuroendocrine differentiation (NED)-associated cancer. Such methods can comprise detecting one or more cells in a sample that express FoxA2 and HIF-1α, wherein detection of cells that express FoxA2 and HIF-1α indicate the presence of or a risk of NED-associated cancer.

Also disclosed are methods of monitoring the risk of metastasis of cancer in a subject. Such methods can comprise detecting one or more cells in a cancer sample that express FoxA2 and HIF-1α, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has reduced the risk of metastasis in the subject. The cancer sample can be a treated cancer sample, wherein the treated cancer sample can be from a subject with cancer that has been treated.

Also disclosed are methods of detecting neuroendocrine differentiation (NED)-associated cancer. Such methods can comprise detecting one or more cells in a cancer sample that express FoxA2 and HIF-1α, wherein detection of cells that express FoxA2 and HIF-1α indicate NED-associated cancer.

Also disclosed are methods of determining a prognosis of a cancer. Such methods can comprise detecting one or more cells in a cancer sample that express FoxA2 and HIF-1α, wherein detection of cells that express FoxA2 and HIF-1α indicate a poor prognosis of the cancer.

Also disclosed are methods of identifying an inhibitor of HIF1α:FoxA2 function or complex formation. Such methods can comprise contacting a compound with HIF-1α or FoxA2; assaying binding of the compound to HIF-1α or FoxA2; and determining if the compound inhibits HIF-1α:FoxA2 function or complex formation. Also disclosed are methods, compounds, and compositions of identifying an inhibitor of HIF1α:FoxA2 complex formation. Such methods can comprise producing a peptide having at least 85% sequence identity to the HIF-1α:FoxA2 interaction domain, and assaying the peptide for the ability to inhibit the formation of a HIF-1α:FoxA2 complex.

Also disclosed are methods of determining a treatment for a cancer. Such methods can comprise detecting one or more cells in a sample that express FoxA2 and HIF-1α, wherein detection of cells that express FoxA2 and HIF-1α indicate as the treatment a NED-associated cancer treatment.

Also disclosed are methods of treating a subject at risk of metastasis of cancer. Such methods can comprise administering to a subject a composition that inhibits expression of one or more HIF-1α:FoxA2-regulated genes. Also disclosed are methods of treating NED-associated cancer. Such methods can comprise administering to a subject a composition that can inhibit the formation of a HIF-1α:FoxA2 complex. Also disclosed are methods of treating NED-associated cancer. Such methods can comprise administering to a subject a composition that inhibits expression of one or more HIF-1α:FoxA2-regulated genes.

Also disclosed are methods of monitoring or determining the effect of treatment of a NED-associated cancer. Such methods can comprise detecting one or more cells in a cancer sample that express FoxA2 and HIF-1α, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has had a positive effect. The cancer sample can be a treated cancer sample, wherein the treated cancer sample can be from a subject with a NED-associated cancer that has been treated,

In some forms of the disclosed methods, FoxA2 and HIF-1α can both be present at or above respective reference levels in the cells that express FoxA2 and HIF-1α, where the presence of FoxA2 and HIF-1α at or above the respective reference levels indicate the presence of or a risk of metastasis of cancer in the subject, the presence of or a risk of neuroendocrine differentiation (NED)-associated cancer, or a combination. In some forms of the disclosed methods, FoxA2 and HIF-1α can both be present at or above respective reference levels in the cells that express FoxA2 and HIF-1α, where the presence of FoxA2 and HIF-1α at or above the respective reference levels indicate the presence of or a risk of metastasis of cancer in the subject. In some forms of the disclosed methods, FoxA2 and HIF-1α can both be present at or above respective reference levels in the cells that express FoxA2 and HIF-1α, where the presence of FoxA2 and HIF-1α at or above the respective reference levels indicates NED-associated cancer. In some forms of the disclosed methods, detection of cells that express FoxA2 and HIF-1α indicates a poor prognosis of the cancer.

In some forms, the disclosed methods can further comprise determining if one or more of the cells that express FoxA2 and HIF-1α also express Hes6, Sox9, Jmjd1a, Plod2, or a combination. In some forms, Hes6, Sox9, Jmjd1a, Plod2, or a combination can be present at or above respective reference levels in the cells that express Hes6, Sox9, Jmjd1a, Plod2, or a combination.

In some forms, the cancer sample can be a prostate sample, a lung sample, a pancreatic sample, or a merkel cell sample. In some forms of the disclosed methods, the cancer can be prostate cancer, lung cancer, pancreatic cancer, or merkel cell carcinoma.

In some forms, the disclosed methods can further comprise treating the subject with a cancer treatment. In some forms, the cancer treatment can be a neuroendocrine differentiation (NED)-associated cancer treatment. In some forms, the subject can have prostate cancer, lung cancer, pancreatic cancer, or merkel cell carcinoma.

A. Detection

Some forms of the disclosed methods make use of detection of certain molecules, components, states, conditions, etc. For example, in some forms, the disclosed methods involve detecting the presence of or a risk of metastasis of cancer in a subject, the method comprising detecting one or more cells in a cancer sample from the subject that express FoxA2 and HIF-1α, wherein detection of cells that express FoxA2 and HIF-1α indicate, for example, a risk of or the presence of metastasis of cancer in the subject, a risk of or the presence of NED-associated cancer in the subject, and/or a poor prognosis of the cancer in the subject. In this example, both cells expressing FoxA2 and HIF-1α and the risk of metastasis of cancer (based on the detection of cells expressing FoxA2 and HIF-1α) are detected. As used herein, “detect” or “detecting” or like terms refer to identifying, determining the presence of, determining the level of, and/or otherwise sensing the presence, non-presence, and/or level and/or amount of an object of detection. Detection generally, but need not, involves detection relative to a specific object of detection distinct from other items, conditions, states, etc. that may be present. At a minimum, all that is required is that the object of detection is detected.

“Risk” or like terms refer to the increased chance, likelihood or the probability that something will occur. A risk can result in a positive or negative event. Some risks can be avoided or altered if certain steps or procedures are followed. As used herein, a risk of metastasis of cancer refers to a risk that a given cancer will or has metastasized that is higher than the average or general risk or probability of such metastasis in the same type of cancer in general. For example, if the average risk or probability of metastasis of prostate cancer in general is a certain level, a risk of metastasis of a cancer detected or identified by the disclosed methods means that the cancer has been identified as having a higher risk or probability of metastasizing. Put another way, the disclosed methods identify cancers where the risk or probability of metastasis is higher than the risk or probability of metastasis in the same type of cancer in general can be anything that increases the likelihood of the cancer spreading. Thus, the risk assessed by the disclosed methods is a higher risk relative to average or base risk.

In some forms of the disclosed methods, FoxA2 and/or HIF-1α are detected. Detection of FoxA2 and/or HIF-1α in a cell or cells indicate that the cell or cells express FoxA2 and/or HIF-1α. Although in the context of the disclosed methods, FoxA2 and HIF-1α are not expressed or are expressed at only low levels in cancers and cancers cells that are distinguished from cancers and cancer cells of interest (those that are at risk of metastasis, neuroendocrine differentiation, etc., for example), expression levels of FoxA2 and HIF-1α that are indicative can also be merely higher than a reference level of FoxA2 and HIF-1α expression. For example, the FoxA2 and HIF-1α can both be present at or above respective reference levels in the cells that express FoxA2 and HIF-1α. The presence of FoxA2 and HIF-1α at or above the respective reference levels indicates, for example, a risk of or the presence of metastasis of cancer in the subject, a risk of or the presence of NED-associated cancer in the subject, and/or a poor prognosis of the cancer in the subject.

As used herein, a “reference level” refers to any level of an item, component, state, condition, etc. that is a base or useful comparison for a test level of the item, component, state, condition, etc. A test level refers to the level measured in, for example, cancers and cancer cells that are being assayed or assessed. In other words, test levels are the “real” levels that are to be compared to a reference level. Respective reference levels refer to reference levels related to the item, component, state, condition, etc. that is being measured. For example, a respective reference level of a test level of FoxA2 is a reference level of

FoxA2. Similarly, a respective reference level of a test level of HIF-1α is a reference level of HIF-1α. Thus, respective reference level is used to match the test level to a reference level relevant to the item, component, state, condition, etc. that is being measured.

The general concept of reference levels is similar to control levels, which can be a form of reference level. What is used as a reference level generally can depend on what is being measured, what is being compared, and/or what is the purpose of the comparison. For example, in the context of the disclosed methods, levels found in healthy cells from the same tissue of the subject as the cancer cells in which test levels are measured, levels found in some cancer cells of the subject other than the cancer cells used for the test level, levels found in cancer cells of the subject at a different time than the cancer cells used for the test level, and levels found in cancer cells in general of the same type as the cancer cells used for the test level can be used as reference levels. As another example, reference levels can be levels found in different tissue of the subject from the cancer cells. As another example, reference levels can be levels found in non-cancerous cells of similar tissue from one or more unrelated individuals. Reference levels can also be those levels found in the general population.

Reference levels can be average levels for any of the suitable sources (such as those discussed above and elsewhere herein). Different reference levels can be particularly useful for some forms of the disclosed methods. For example, levels found in cancer cells of the subject at a different time than the cancer cells used for the test level are a useful type of reference level for assessing the effectiveness of an intervening treatment, to monitor the progress of the cancer, to monitor the progress of treatment, and to assess the prognosis or changing prognosis of the cancer.

The term “prognosis” encompasses predictions about the likely course of disease or disease progression, particularly with respect to likelihood of disease remission, disease relapse, tumor recurrence, metastasis, and death. “Good prognosis” refers to the likelihood that the subject will have a less aggressive cancer (less differentiation and metastasis, for example), and/or a more positive or better outcome or result. “Poor prognosis” refers to the likelihood that the subject will suffer a relapse, recurrence, and/or worsening of the underlying cancer or tumor, metastasis, death, and/or other negative or worse outcome or result. Prognosis is assessed using some forms of the disclosed methods and is thus determined at the time when the assessment is made; for example, when the relevant assay or method is performed, such as before treatment, during treatment, and after treatment. The time frame for which the prognosis and outcome is relevant can be any suitable time frame. For example, the time frame for assessing prognosis and outcome can be less than one year, one, two, three, four, five, six, seven, eight, nine, ten, fifteen, twenty or more years. In some forms, the relevant time for assessing prognosis or disease-free survival time begins with the surgical removal of the tumor or suppression, mitigation, or inhibition of tumor growth. Thus, in some forms, a “good prognosis” refers to the likelihood, for example, that the cancer of the subject will not metastasize for a period of at least one, two, three, four, five, or ten. As another example, in some forms, a “good prognosis” refers to the likelihood, for example, that a prostate cancer patient will remain free of the underlying cancer or tumor for a period of at least one, two, three, four, five, or ten years. In some forms, a “bad prognosis” refers to the likelihood, for example, that the cancer of the subject will metastasize within less than one, two, three, four, five, or ten. As another example, a “bad prognosis” refers to the likelihood, for example, that a lung cancer patient will experience disease relapse, tumor recurrence, metastasis, and/or death within less than one, two, three, four, five, or ten. Time frames for assessing prognosis and outcome provided above are illustrative and are not intended to be limiting.

Upon determining if one or more of the cells express FoxA2 and HIF-1α, it can also be determined if the cells express, for example, Hes6, Sox9, Jmjd1a, Plod2, or a combination. In this context, Hes6, Sox9, Jmjd1a, Plod2, or a combination can be present at or above respective reference levels in the cells that express Hes6, Sox9, Jmjd1a, Plod2, or a combination. Hes6, Sox9, Jmjd1a, and Plod2 are examples of HIF-1α:FoxA2 regulated genes. HIF-1α:FoxA2 regulated genes are the subset of genes that are regulated by the HIF-1α transcription factor in which transcription is further stimulated by FoxA2.

The item, component, state, condition, etc. that is being measured (HIF-1α and FoxA2, for example) can be detected, measured, or assessed using any suitable method or technique. For example, HIF-1α and FoxA2 can be detected using any suitable method, technique and compositions. Many general assays and assay compositions are known and can be used or adapted to detect cells that express HIF-1α and FoxA2. Certain methods and compositions may be better suited to detecting HIF-1α and FoxA2 expression, HIF-1α and FoxA2 expression levels, and/or cells expressing HIF-1α and FoxA2. Those of skill in the art are aware of how to apply known techniques for these purposes. Some useful methods, techniques and compositions are discussed below.

By sample or like terms is meant a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components. A cancer sample is a sample made up of or derived from a cancer or cancer cells. A cell can be in vitro. Alternatively, a cell can be in vivo and can be found in a subject. A “cell” can be a cell from any organism including, but not limited to, a bacterium.

1. Labels

Detection, and other aspects of the disclosed methods, compounds, and compositions, can be aided by the use of labels. Useful labels include any molecule that can be associated with nucleic acid, protein, or other appropriate molecule, directly or indirectly, and which results in a measurable, detectable signal, either directly or indirectly. Many such labels are known to those of skill in the art. Examples of labels suitable for use in the disclosed method include radioactive isotopes, fluorescent molecules, phosphorescent molecules, enzymes, antibodies, and ligands.

Labels can include, for example, a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is useful as they can be detected at very low amounts. Furthermore, in the case where multiple antigens are reacted with a single array or assay, each antigen can be labeled with a distinct fluorescent compound for simultaneous detection. Labeled spots on an array or in an assay can be detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4- Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4- I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis- BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson- ; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade B1ue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD- Lipophilic Tracer; DiD (Di1C18(5)); DIDS; Dihydorhodamine 123 (DHR); Dil (Di1C18(3)); I Dinitrophenol; DiO (DiOC18(3));

DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; ; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO- I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl)quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO- PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as radionuclides can be incorporated into or attached directly to any of the compounds described herein by, for example, halogenation. Examples of useful radionuclides include, but are not limited to, tritium, iodine-125, iodine-131, iodine-123, iodine-124, astatine-210, carbon-11, carbon-14, nitrogen-13, fluorine-18. Radionuclides can also be attached to a linking group or bound by a chelating group, which is then attached to the compound directly or by means of a linker. Examples of radionuclides useful in this aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In the context of antibody detection methods, direct labeling can involve the detecting antibody (the antibody for the molecule of interest) or detecting molecule (the molecule that can be bound by an antibody to the molecule of interest) including a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Direct and indirect labeling can be used for other modes of labeling and detection. The relationships between the molecule to be detected, the molecule that binds the molecule to be detected, and the molecule that carries the label, can be organized by analogy to the description above and elsewhere herein regarding antibody detection. For example, in direct labeling, the label can be attached to the molecule that binds the molecule to be detected. In indirect labeling, the label can be attached to a molecule that can bind to or interact with, directly or indirectly, the molecule that binds the molecule to be detected.

2. Immunohistochemistry

Disclosed herein are immunohistochemistry methods that can be used to detect, for example, HIF-1α and FoxA2 in cancer cells for use in the methods disclosed herein. In some aspects, the method can comprise immunolabeling HIF-1α and FoxA2 in cancer tissue and creating a digital image of the immunolabeling. In some aspects, the digital image can be analyzed to count the percentage of immunolabled cells. Other adaptations of standard immunohistochemistry methods are known and can be used in the disclosed methods.

Immunohistochemistry or IHC refers to the process of localizing proteins in cells of a tissue section exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used in basic research to understand the distribution and localization of biomarkers and differentially expressed proteins in different parts of a biological tissue.

Visualizing an antibody-antigen interaction can be accomplished in a number of ways. In the most common instance, an antibody is conjugated to an enzyme, such as peroxidase, that can catalyze a color-producing reaction (see immunoperoxidase staining) Alternatively, the antibody can also be tagged to a fluorophore, such as FITC, rhodamine, Texas Red, Alexa Fluor, or DyLight Fluor(see immunofluorescence). The latter method is of great use in confocal laser scanning microscopy, which is highly sensitive and can also be used to visualize interactions between multiple proteins.

There are two strategies used for the immunohistochemical detection of antigens in tissue, the direct method and the indirect method. In both cases, the tissue is treated to rupture the membranes, usually by using a kind of detergent such asTriton X-100. Some antigen also need additional step for unmasking, resulting in better detection results.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. (The secondary antibody must be against the IgG of the animal species in which the primary antibody has been raised.) This method is more sensitive due to signal amplification through several secondary antibody reactions with different antigenic sites on the primary antibody. The second layer antibody can be labeled with a fluorescent dye or an enzyme.

3. Immunoassay

Disclosed herein are immunoassays that can be used, for example, to detect HIF-1α and FoxA2 in cancer cells for use in the methods disclosed herein. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/ FLAP).

In general, immunoassays involve contacting a sample suspected of containing a molecule of interest (such as the disclosed cancer samples) with an antibody to the molecule of interest or contacting an antibody to a molecule of interest (such as antibodies to HIF-1α and FoxA2) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed HIF-1α and FoxA2 or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

Immunoassays that involve the detection of a substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

An immunoassay that uses electrophoresis that can be used with the disclosed methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Fan Assay, Precipitin Assay, Radioimmune Precipitin Assay; Radioimmunoprecipitation Analysis; Radioimmunoprecipitation Analysis, and Radioimmunoprecipitation Analysis.

Also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995;U.S. Pat. No. 4,376,110, each of which is incorporated herein by reference in its entirety and specifically for teachings regarding ELISA methods.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISAs, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Conditions effective to allow immunecomplex (antigen/antibody) formation refers to conditions that include, for example, diluting the antigens and antibodies with solutions such as BSA, bovine gamma globulin (BGG) and phosphate buffered saline (PBS)/Tween so as to reduce non-specific binding and to promote a reasonable signal to noise ratio.

Effective or suitable conditions generally also include that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents—which are usually antibodies but can also be alternative protein scaffolds, peptides or nucleic acid aptamers—can be used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays can be used to quantitate and compare the levels of proteins in different samples in health and disease, i.e. protein expression profiling. Proteins other than specific ligand binders can be used in the array format for in vitro functional interaction screens such as protein-protein, protein-DNA, protein-drug, receptor-ligand, enzyme-substrate, etc. The capture reagents themselves can be selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g., where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include color coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization can be used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used and can be sued with the disclosed methods. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labeling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

4. Separation Methods

Disclosed herein are separation methods that can be used, for example, to detect the number or percentage of cells with detectable HIF-1α and FoxA2 expression for use in the methods disclosed herein. Cells with detectable HIF-1α and FoxA2 expression can be isolated by a fluorescence activated cell sorting (FACS), protein-conjugated magnetic bead separation, specific gene expression patterns (using RT-PCR), or specific antibody staining

Cells can be selected based on, for example, light-scatter properties as well as their expression of various cell surface antigens. Various techniques can be employed to separate the cells with detectable HIF-1α and/or FoxA2 expression. Monoclonal antibodies are particularly useful. The antibodies can be attached to a solid support to allow for crude separation. The separation techniques employed should maximize the retention of viability of the fraction to be collected.

Procedures for separation can include, for example, magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique.

The antibodies can be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. If the viability of the cells is important, the technique to be employed can be one that is not unduly detrimental to the viability of the remaining cells.

5. Nucleic Acid Detection

Disclosed herein are methods for detecting and determining the abundance of, for example, HIF-1α and FoxA2 nucleic acids, such as, for example, mRNA in, for example, a total or poly(A) RNA sample from cancer cells. Any suitable technique can be used for such detection. For example, specific mRNAs can be detected using Northern blot analysis, nuclease protection assays (NPA), in situ hybridization, or reverse transcription-polymerase chain reaction (RT-PCR). Myriad other techniques have been developed and are known for detecting, identifying, quantitating, etc. specific nucleic acids. Such techniques can be used to detect nucleic acids in the disclosed methods.

Each of these techniques can be used to detect specific RNAs and to precisely determine their expression level. Northern analysis is useful for providing information about transcript size. NPAs are useful to simultaneously examine multiple messages. In situ hybridization is useful for localizing expression of a particular gene within a tissue or cell type. RT-PCR is useful for sensitive detection and quantitation of gene expression.

Northern analysis is useful for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

RT-PCR makes it possible to detect the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR. As with NPAs, RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) can vary in expression. Additionally, most common internal controls are expressed at much higher levels than the mRNA being studied. Preferably, all products of the PCR reaction can be analyzed in the linear range of amplification, which becomes difficult for transcripts of widely different levels of abundance.

Competitive RT-PCR can be used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

i. Primers and Probes

The disclosed methods can make use of nucleic acids for various purposes. For example, probes and primers can be used in detection and quatitation techniques. For example, disclosed are nucleic acids that can be used to detect, for example, HIF-1α and FoxA2 in cancer cells for use in the methods disclosed herein. For example, disclosed are compositions including primers and probes, which are capable of interacting with HIF-1α or FoxA2 transcripts.

Primers can be used to support DNA (e.g., cDNA) replication and/or amplification reactions. Typically the primers will be capable of being extended in a sequence-specific manner. Extension of a primer in a sequence-specific manner includes any methods where the sequence and/or composition of the nucleic acid molecule to which the primer is hybridized or otherwise associated directs or influences the composition or sequence of the product produced by the extension of the primer. Extension of the primer in a sequence-specific manner can include, but is not limited to, PCR, DNA sequencing, DNA extension, DNA polymerization, RNA transcription, or reverse transcription. Techniques and conditions that amplify the primer in a sequence specific manner are particularly useful. In certain embodiments the primers can be used for DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids can be, for example, any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some forms, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Primers for the HIF-1α or FoxA2 genes or transcripts can be used, for example, to produce an amplified DNA product that contains a region of the HIF-1α or FoxA2 genes or transcripts or the complete genes or transcripts. In general, typically the size of the product will be such that the size can be accurately determined to within 3, or 2 or 1 nucleotides.

This amplified product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some forms, the amplified product can be less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The disclosed nucleic acids can be made up of, for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### B. Risk of Metastasis and Prognosis of Cancer

Disclosed are methods of detecting, determining, identifying, monitoring, treating, etc. risk, such as risk of metastasis and risk of NED-associated cancer. Also disclosed are methods of detecting, determining, identifying, monitoring, etc. prognosis of a subject, cancer, NED-associated cancer, or metastasis. It has been discovered that expression of FoxA2 and HIF-1α is a determinant of risk of metastasis and NED-associated cancer and thus to a poorer prognosis for the subject and the cancer. Relevant to this, also disclosed are methods of detecting, determining, monitoring, etc. expression of FoxA2 and HIF-1α, HIF1α:FoxA2-regulated genes, or a combination. Also disclosed are methods of detecting, determining, identifying, monitoring, etc. metastasis, cancer, NED-associated cancer, HIF1α:FoxA2 complex, HIF1α:FoxA2 function, HIF1α:FoxA2 complex formation, or a combination.

As an example, disclosed are methods of determining a prognosis of a cancer, the method comprising detecting one or more cells in a cancer sample that express FoxA2 and HIF-1α, where detection of cells that express FoxA2 and HIF-1α indicates a poor prognosis of the cancer.

Such detection, determination, identification, selection, monitoring, etc. can be accomplished by detecting, determining, identifying, monitoring, etc. relevant proteins, genes, nucleic acids, compounds, compositions, molecules, etc. Components and effects of the expression of FoxA2 and HIF-1α, such as HIF1α:FoxA2 complex, HIF1α:FoxA2 function, HIF1α:FoxA2 complex formation, and HIF1α:FoxA2-regulated genes can be detected, determined, identified, monitored, etc. to make or support detecting, determining, identifying, monitoring, etc. risk and prognosis as for expression of FoxA2 and HIF-1α.

Metastasis is a well-know concept and feature in cancer. Without being limited, metastasis of cancer can refer to the spreading of cancer. For example, the cancer spreads from the original tissue to an different tissue/organ in the body. For instance, metastasis of prostate cancer can occur when the cancer spreads to the lymph nodes. Generally, and consistent with the discoveries and disclosures herein, metastasis can occur, can be based on, and can involve genetic, biochemical, and/or physiological changes in cancer cells that allow or support cancer cells migrating to other parts of the subject's body and forming new foci of cancer.

### C. Identifying

Disclosed are methods of identifying, selecting, detecting, determining, etc. inhibitors of HIF1α, FoxA2, HIF1α expression, FoxA2 expression, HIF1α:FoxA2 complex, HIF1α:FoxA2 function, and/or HIF1α:FoxA2 complex formation. Also disclosed are methods of identifying, selecting, detecting, determining, etc. compounds, proteins, compositions, molecules, etc. for affecting metastasis, cancer, NED-associated cancer, HIF1α, FoxA2, HIF1α expression, FoxA2 expression, HIF1α:FoxA2 complex, HIF1α:FoxA2 function, and/or HIF1α:FoxA2 complex formation. Also disclosed are methods of identifying, selecting, detecting, determining, etc. compounds, proteins, compositions, molecules, etc. for treating metastasis, cancer, NED-associated cancer, HIF1α, FoxA2, HIF1α expression, FoxA2 expression, HIF1α:FoxA2 complex, HIF1α:FoxA2 function, and/or HIF1α:FoxA2 complex formation. Such inhibitors, compounds, proteins, compositions, molecules, etc. can be used for treating subjects and cancers at risk of metastasis or NED-associated cancer.

For example, disclosed are methods of identifying an inhibitor of HIF1α:FoxA2 function or complex formation, the method comprising contacting a compound with HIF-1α or FoxA2; assaying binding of the compound to HIF-1α or FoxA2; and determining if the compound inhibits HIF-1α:FoxA2 function or complex formation. The disclosed compound can be, for example, a peptide having at least 60%, 65%, 70%, 75%, 80%, 85%, 90%, 92%, 94%, 95%, 96%, 97%, 98%, or 99% sequence identity to HIF-1α:FoxA2 interaction domain. Also disclosed are methods of identifying an inhibitor of HIF1α:FoxA2 complex formation, the method comprising producing a peptide having, for example, at least 85% sequence identity to the HIF-1α:FoxA2 interaction domain, and assaying the peptide for the ability to inhibit the formation of a HIF-1α:FoxA2 complex.

### D. Treatment

Disclosed are methods of treating metastasis, cancer, NED-associated cancer, HIF1α, FoxA2, HIF1α expression, FoxA2 expression, HIF1α:FoxA2 complex, HIF1α:FoxA2 function, and/or HIF1α:FoxA2 complex formation. Also disclosed are methods of identifying, selecting, detecting, monitoring, determining, etc. treatments for metastasis, cancer, NED-associated cancer, HIF1α, FoxA2, HIF1α expression, FoxA2 expression, HIF1α:FoxA2 complex, HIF1α:FoxA2 function, and/or HIF1α:FoxA2 complex formation. In general, detection, determination, identification, etc. that a subject or cancer has or is at risk of metastasis or NED-associated cancer, treatment of the subject or cancer can be identified, selected, chosen, etc. to be suited to metastatic cancer or NED-associated cancer. As another example, determination that a subject or cancer has or is at risk of metastasis or NED-associated cancer, a more aggressive treatment or treatment regime can be used.

For example, disclosed are methods of treating a subject having or at risk of metastasis of cancer, the method comprising administering to a subject a composition that inhibits expression of one or more HIF-1α:FoxA2-regulated genes. HIF-1α:FoxA2 regulated genes can include but are not limited to Hes6, Sox9, jmjd1a or Plod2. These are genes that require the transcription factors HIF-1α and FoxA2 for expression.

For example, disclosed are methods of treating NED-associated cancer, the method comprising administering to a subject a composition that inhibits expression of one or more HIF-1α:FoxA2-regulated genes. Also disclosed are methods of a subject having or at risk of treating NED-associated cancer, the method comprising administering to the subject a composition that inhibits expression of one or more HIF-1α:FoxA2-regulated genes. The NED-associated cancer can comprise one or more cells that express FoxA2 and HIF-1α. The subject can have a risk of metastasis of the NED-associated cancer. The risk of metastasis can be indicated by, for example, one or more cancer cells that express FoxA2 and HIF-1α. The risk of metastasis can also be indicated by one or more of the cancer cells that express FoxA2 and HIF-1α also expressing Hes6, Sox9, Jmjd1α, Plod2, or a combination.

The subjects and cancers to be treated as disclosed herein can be identified are selected in a variety of ways. For example, the disclosed methods of treatments can comprise the subject being diagnosed with a NED-associated cancer prior to treatment. As another example, the subject can have, for example, prostate cancer, lung cancer, pancreatic cancer or merkel cell carcinoma. As another example, the subject can have been identified as having a risk of metastasis by, for example, detection of one or more cancer cells that express FoxA2 and HIF-1α. As another example, the subject can have suffered from cellular hypoxia such as mild cellular hypoxia. This can occur, for example, in larger tumor masses. Cellular hypoxia refers to less than the normal physiologic levels of oxygen supplied to a cell or tissue. Mild cellular hypoxia refers to 2-6% oxygen.

As another example, the cancer to be treated can comprise, for example, one or more cells that express FoxA2 and HIF-1α. As another example, the cancer to be treated can comprise, for example, one or more cancer cells that express FoxA2 and HIF-1α can also express Hes6, Sox9, Jmjd1a, Plod2, or a combination.

Also disclosed are methods of determining a treatment for a cancer. For example, the method can comprise detecting one or more cells in a sample that express FoxA2 and HIF-1α, where detection of cells that express FoxA2 and HIF-1α indicates as the treatment a NED-associated cancer treatment. NED-associated cancer treatments are those treatments suited for or targeted to characteristics of NED-associated cancer. For example, NED-associated cancer treatments can be cancer treatments particularly suited for or targeted to features or aspects of NED-associated cancer. For example, treatments particularly suited for or targeted to metastasis, metastatic cancer, neuroendocrine differentiation, neuroendocrine phenotype, Siah2, dependence on Siah2, FoxA2, FoxA2 expression, HIF-1α, HIF-1α expression, HIF-1α:FoxA2 complex, formation of HIF-1α:FoxA2 complex, HIF-1α:FoxA2-regulated genes, expression of HIF-1α:FoxA2-regulated genes, Hes6, Sox9, Jmjd1a, Plod2, expression of Hes6, expression of Sox9, expression of Jmjd1a, expression of Plod2, and other HIF-1α:FoxA2-regulated genes and the like can be NED-associated cancer treatments. Forms of NED-associated cancer treatments can include but are not limited to radiotherapy, chemotherapy, surgery, drug therapy and hormone therapy.

By “treating” or “treatment” is meant the medical management of a patient with the intent to cure, ameliorate, stabilize, or prevent a disease, pathological condition, or disorder. These terms include active treatment, that is, treatment directed specifically toward the improvement of a disease, pathological condition, or disorder, and also includes causal treatment, that is, treatment directed toward removal of the cause of the associated disease, pathological condition, or disorder. These terms can mean that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertently cause harm. In addition, these terms include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. These terms do not require that the treatment in fact be effective to produce any of the intended results. It is enough that the results are intended.

As used herein, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

### E. Monitoring

The association of expression of FoxA2 and HIF-1α with a risk of metastasis and a poor prognosis make these and related features useful for monitoring the progress and prognosis of the subject and cancer. In the same way, expression of FoxA2 and HIF-1α and related features are useful for monitoring the progress and prognosis of treatment of the subject and cancer. Thus, for example, disclosed are methods of monitoring or determining the effect of treatment of a NED-associated cancer, the method comprising detecting one or more cells in a cancer sample that express FoxA2 and HIF-1α, where the cancer sample is a treated cancer sample, where the treated cancer sample is from a subject with a NED-associated cancer that has been treated, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has had a positive effect.

The effect of a treatment can be positive or negative or the treatment can have no effect. The effect of a treatment can be measured or determined in a variety of ways. A positive effect can be any effect which shows an improvement in the subject or the cancer compared to that feature pre-treatment or at an earlier time during treatment. For example, a positive effect can be reduction in tumor size, reduction in rate/speed of metastasis, reduction of levels of HIF-1α or FoxA2, reduction in number of cells expressing HIF-1α and/or FoxA2. Negative effects of a treatment can be, but are not limited to, any effect which shows a detriment in the subject or the cancer compared to that feature pre-treatment or at an earlier time during treatment. For example, an increase in tumor size, increase in number of cells expressing HIF-1α and/or FoxA2, increase in levels of HIF-1α and/or FoxA2 or increase in metastasis can be negative effects and/or indicators of negative effects. A treatment can also have no effect (that is, no positive or negative effect). This can be shown, for example, by the absence of positive and negative effects. For example, no effect of treatment can be indicated by, for example, the number of cells expressing HIF-1α and/or FoxA2, level of HIF-1α and/or FoxA2, etc. remaining the same after treatment as compared to prior to treatment, tumor size remaining the same after treatment compared to prior to treatment or at an earlier time during treatment.

Also disclosed are methods of monitoring the risk of metastasis of cancer in a subject, the method comprising detecting one or more cells in a cancer sample that express FoxA2 and HIF-1α, where the cancer sample is a treated cancer sample, where the treated cancer sample is from a subject with cancer that has been treated, and comparing the number of cells, fraction of cells, level of FoxA2, level of HIF-1α, or a combination in the treated cancer sample with the number of cells, fraction of cells, level of FoxA2, level of HIF-1α, or a combination in a cancer sample from the same subject prior to or earlier during the treatment, wherein a reduction in the number of cells, fraction of cells, level of FoxA2, level of HIF-1α, or a combination indicates that the treatment has reduced the risk of metastasis in the subject.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a cancer sample” includes a plurality of such cancer samples, reference to “the cancer sample” is a reference to one or more cancer samples and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

Those skilled in the art will recognize, or be able to ascertain using no more than routine experimentation, many equivalents to the specific embodiments of the method and compositions described herein. Such equivalents are intended to be encompassed by the following claims.

### Examples

### A. Siah2-Dependent Concerted Activity of HIF & FoxA2 Regulates Formation of Neuroendocrine Phenotype & Neuroendocrine Prostate Tumors

Neuroendocrine (NE) phenotype, seen in >30% of prostate adenocarcinomas (PCa), and NE prostate tumors are implicated in aggressive prostate cancer. Formation of NE prostate tumors in the TRAMP mouse model was suppressed in mice lacking the ubiquitin ligase Siah2, which regulates HIF-1α availability. Cooperation between HIF-1α and FoxA2, a transcription factor expressed in NE tissue, promotes recruitment of p300 to transactivate select HIF-regulated genes, Hes6, Sox9 and Jmjd1a. These HIF-regulated genes are highly expressed in metastatic PCa and required for hypoxia-mediated NE phenotype, metastasis in PCa and the formation of NE tumors. Tissue-specific expression of FoxA2 combined with

Siah2-dependent HIF-1α availability enables a transcriptional program required for NE prostate tumor development and NE phenotype in PCa.

Prostate adenocarcinomas (PCa) with neuroendocrine (NE) phenotype and NE prostate tumors are associated with poor prognosis and androgen independence. Demonstrated herein is that formation of NE tumors and metastasis of PCa require the ubiquitin ligase Siah2. Through its role in the control of HIF-1α availability, Siah2 enables cooperation between HIF-1α and the NE-specific transcription factor FoxA2. Genes induced by HIF-1α/FoxA2 cooperation are expressed in NE lesions and in metastatic human PCa and required for formation of the NE phenotype, for metastasis of human PCa, and for the development of NE tumors. Tissue-specific cooperation between transcription factors that promote NE phenotype and prostate tumor development offers a paradigm for the development, progression and potential targeting of aggressive prostate tumors.

1. Results

i. Attenuated Formation of Prostate NE Carcinoma in Siah2-Null TRAMP Mice.

The TRAMP mouse model, in which prostate-specific expression of SV40 T antigen results in prostate tumors that metastasize to lymph nodes, lung and liver (Gingrich et al., 1996), was employed to assess the possible role of Siah in tumor growth and metastasis. Analysis of 8-month-old TRAMP mice with different Siah2 background (TRAMP/Siah2^{-/-}, TRAMP/Siah2^{+/-}, and TRAMP/Siah2^{+/+}) revealed that the majority developed prostate masses (FIG. 1A). Most primary masses were composed of benign proliferations of stroma and epithelium with atypical epithelial hyperplasia (AH) in TRAMP/Siah2^{-/-} mice, compared with a preponderance of NE carcinoma in the TRAMP/Siah2^{-/-} and TRAMP/Siah2^{+/+} mice (FIG. 1C). Although TRAMP AH have been referred to as adenocarcinoma in some literature (e.g., Gingrich et al., 1996), they are also referred to as TRAMP AH in lieu of clear discrimination, detailed in Chiaverotti et al., (2008). NE carcinomas were identified by morphology and expression of the well-established NE markers synaptophysin, neuron-specific enolase (NSE) and FoxA2, consistent with the notion that the TRAMP tumors are primarily of NE origin (Chiaverotti et al., 2008; Mirosevich et al., 2006). In clear contrast, FoxA2, synaptophysin and NSE were undetectable in normal prostate glands or in AH.

Since Siah1 also contributes to the regulation of PHD and consequently HIF availability (Nakayama et al., 2004; Qi et al., 2008), Siahl a function in prostate tumor formation was also evaluated. Siah2 and Siah1a doubly homozygous mutant mice are non-viable (Frew et al., 2003). A TRAMP/Siah1a^{+/-}Siah2^{-/-} mouse line was established. TRAMP/Siah1a^{+/-}Siah2^{-/-} mice showed a prostate tumor incidence similar to that seen in TRAMP/Siah2^{-/-} mice (FIG. 1A) but lacked NE tumors (FIG. 1C).

In the TRAMP mice, the early NE tumor lesions develop in the ventral prostate after 3 months and are recognized as foci that express NE markers. To examine the effect of Siah2 on the development of early stage NE tumors, 5-month-old mice were analyzed. NE foci were identified in 3/6 control mice but in none of the 10 TRAMP/Siah2^{-/-} mice, the difference is statistically significant. These data reveal that in the TRAMP model Siah is required for development of NE carcinomas.

AH predominantly develops in the dorsal lateral lobe of prostate and is identifiable in 1-month-old TRAMP mice (Chiaverotti et al., 2008). To evaluate whether Siah also involved in the progression of AH, the dorsal prostate lobes from 1- and 3-month-old mice were analyzed and found that lack of Siah2 delayed the progression from a normal prostate gland to the early and medium stages AH in 1-month-old mice and into the late stage AH in the 3-month-old mice (FIG. 8).

ii. Altered HIF-1α Expression Correlates with Reduced Cell Proliferation and Enhanced Cell Death in Primary NE Tumor from TRAMP/Siah^{-/-} Mice

Consistent with Siah2 regulates HIF-1α availability, HIF-1α level is reduced in the very few NE carcinomas observed and in AH from TRAMP/Siah2^{-/-} mice compared with TRAMP/Siah2^{+/-} mice. HIF-2α staining was also reduced in AH from TRAMP/Siah2^{-/-} mice, whereas HIF-2α was undetectable in NE carcinoma from any genotype. These findings are also consistent with the observation that HIF-1α but not HIF-2α is co-expressed with NE markers in prostate cancers (Monsef et al., 2007).

Potential changes in proliferation, apoptosis and angiogenesis were evaluated using PCNA, TUNEL/active caspase-3 and CD31, respectively. NE tumors, but not AH, from TRAMP/Siah2^{-/-} mice showed reduced cell proliferation and increased apoptosis compared with those seen in TRAMP/Siah2^{+/-} mice (FIG. 1C, Table 1). Vascular density was similar in the two strains in both the NE tumor (Table 1) and AH (FIG. 1C, Table 1). Hence, loss of Siah and consequent downregulation of HIF levels appear to specifically govern proliferation and cell survival of NE tumors.

TABLE 1
(related to FIG. 1) Quantification of IHC staining.

| NE carcinoma | Siah2+/− | Siah2−/− | p value |
|---|---|---|---|
| PCNA (%) | 67.8 ± 11.3 | 12.8 ± 6.3 | <1 × 10⁻⁵ |
| Caspase-3 (%) | 1.5 ± 0.7 | 9.8 ± 4.1 | <0.005 |
| CD31 (#/field) | 7.8 ± 1.5 | 6.0 ± 1.6 | >0.05 |
| AH | | | |
| PCNA (%) | 85.4 ± 5.7 | 90.4 ± 4.0 | >0.1 |
| TUNEL | 0.6 ± 0.5 | 0.8 ± 0.8 | >0.1 |
| (#/field) | | | |
| CD31 (#/field) | 9.6 ± 2.0 | 11 ± 2.7 | >0.1 |

NE and AH sections derived from TRAMP mice on the background Siah2+/− or Siah2−/− were subjected to IHC staining of PCNA, active caspase-3, TUNEL or CD31.
The percentage of PNCA (+) or active caspse-3 (+) nucleus was calculated using the nuclear quantification algorism of Scanscope on 10 random high-power fields.
The # of CD31 (+) vessels or TUNEL (+) cells was manually counted on 10 random high-power fields.

To directly assess a possible role for Siah2 and HIF-1α in tumorigenesis of TRAMP cells, TRAMP-C cells derived from TRAMP tumors were analyzed (Foster et al., 1997). These cells likely represent NE tumor-derived cells as they express multiple NE specific transcription factors (FIG. 4A). The PHYL peptide binds to Siah's substrate recognition site (House et al., 2003) and attenuates Siah2's effect on PHD1/3, thus reduces HIF-1α levels under hypoxia (Moller et al., 2009; Qi et al., 2008). Expression of the PHYL peptide in TRAMP-C cells effectively abolished their ability to form tumors (FIGS. 1D, 1E), consistent with the finding that NE tumors do not form in TRAMP/Siah1a^{+/-} Siah2^{-/-} mice (FIG. 1C). Forced HIF-1α expression in TRAMP-C cells expressing PHYL peptide by transfection (FIG. 1B) partially recovered their ability to form tumors (FIGS. 1D, 1E). These data support the role of Siah2, in part through its regulation of HIF-1α levels, in formation of NE prostate tumors.

iii. Reduced Metastasis in TRAMP/Siah^{-/-} Mice

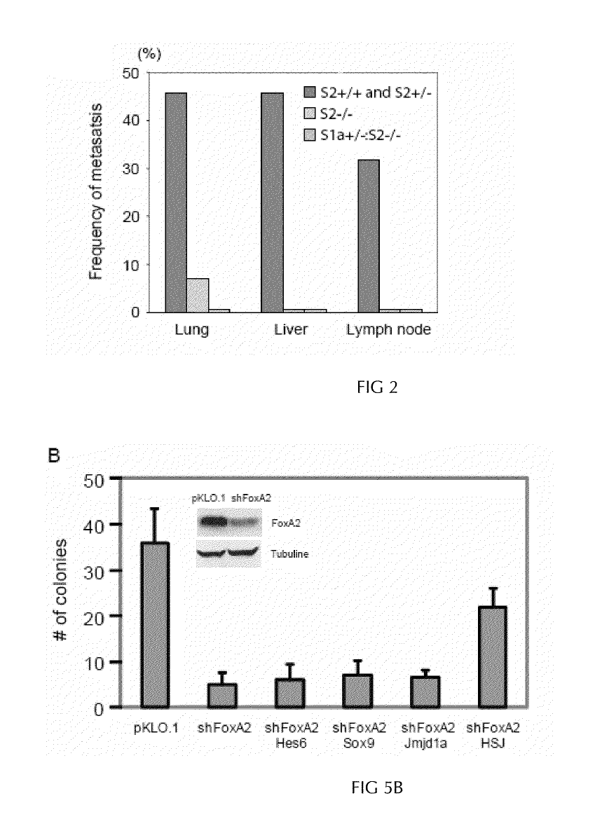

Metastatic lesions in liver, lung and lymph nodes of TRAMP mice were identified as NE carcinomas, based on morphology and FoxA2/synaptophysin expression. However, both the frequency and size of metastatic lesions were significantly reduced (6 fold) in the lung and were not found in liver and lymph nodes of TRAMP/Siah2^{-/-} mice, compared with TRAMP/Siah2^{+/-} or TRAMP/Siah2^{-/-} animals (FIG. 2). Furthermore, no metastases in TRAMP/Siah1a^{+/-} Siah2^{-/-} mice was found (FIG. 2). The very few lung metastases observed in TRAMP/Siah2^{-/-} mice were smaller, showed reduced cell proliferation and enhanced cell death. These findings point to the role of Siah2 in TRAMP tumor metastasis.

iv. FoxA2 Stimulates HIF Transcriptional Activity

Since the NE-specific transcription factor FoxA2 is co-expressed with HIF-1α protein in nuclei of NE carcinoma cells, the possibility that these transcription factors cooperate with each other was tested. While expression of exogenous FoxA2 in TRAMP-C cells did not alter the expression of an HRE-linked luciferase construct (HRE-Luc), expression of exogenous HIF-1α alone elicited a modest increase in HRE-Luc activity as expected (FIG. 3A). Significantly, co-expression of HIF-1α and FoxA2 led to a 6-fold increase in luciferase activity over expression of HIF-1α alone (FIG. 3A). Co-expression of IPAS, a spliced form of HIF-3α with potent dominant-negative activity towards all HIFs (Makino et al., 2002) or inhibiting HIF-1α expression by expressing S2RM (a dominant-negative form of Siah2) or PHYL abolished FoxA2-potentiated HRE-Luc activity (FIG. 3A). Importantly, knockdown of FoxA2 caused an approximately 40% reduction in HRE-Luc activity under hypoxia, although the degree of inhibition was lower than that seen using HIF-1α or HIF-1β siRNA (FIGS. 3B, 9A). These data establish a role for FoxA2 in enhancing HIF-mediated transcriptional activity. In contrast, HIF-1α did not stimulate FoxA2 transcription activity, indicating that HIF/FoxA2 transcriptional synergy may be restricted to the context of an HRE. Notably, FoxA1 did not increase HRE-Luc activity in the presence of HIF-1α.

The FoxA2 domains required for cooperation with HIF-1α were then mapped. FoxA2 fragments consisting of the N-terminal transactivation domain (N-TAD), the central forkhead domain, or the C-terminal transactivation domain (C-TAD) were generated and evaluated for their effect on FOXA- and HIF-1α-dependent transcription activity (FIG. 9B). FoxA2 mutants lacking either the N-TAD or C-TAD exhibited FOXA-dependent transcriptional activity similar to that of the wild-type FoxA2 (FIG. 3C), indicating that one transactivation domain is sufficient for FoxA2 transcriptional activity. FoxA2 mutants lacking the C-TAD promoted HIF-dependent transcriptional activity to a level similar to wild-type FoxA2, whereas the FoxA2 mutant lacking the N-TAD was much less effective (FIG. 3D). The FoxA2 C-terminus, which contains intrinsic chromatin remodeling activity (Cirillo et al., 2002), was dispensable for HRE activation (construct m-2 in FIGS. 3D, 9B) thereby excluding the role of chromatin remodeling activity in FoxA2 cooperation with HIF. These data indicate that the N-TAD and forkhead domains of FoxA2 are required to stimulate HIF-mediated transcriptional activity.

v. HIF Interacts with FoxA2.

Co-expression of HA-FoxA2 or HA-FoxA1 with Flag-HIF-1α in 293T cells followed by immunoprecipitation of Flag-HIF-1α identified HA-FoxA2,but not HA-FoxA1, as an HIF-associated protein (FIG. 3E), consistent with the effect of FoxA2 but not FoxA1 on HIF-dependent transcriptional activity. Importantly, endogenous FoxA2 was co-precipitated with endogenous HIF-1α (FIG. 3F) or HIF-1β (FIG. 3G) in TRAMP-C cells maintained under hypoxia. The association of HIF-1β with FoxA2 was abolished following HIF-1α knockdown (FIG. 3G), indicating that HIF-1α recruits FoxA2 to the HIF complex under hypoxia. Furthermore, in vitro binding between purified His-FoxA2 and in vitro translated Flag-HIF-1α confirmed their direct interaction (FIG. 3H).

In vitro binding using truncation mutants of HIF-1α (FIG. 9C) and FoxA2 (FIG. 9B) identified the bHLH-PAS domain of HIF-1α (FIG. 3H) and the N-TAD of FoxA2 (FIG. 31) as the minimal regions mediating the HIF-1α-FoxA2 interaction. Although the bHLH-PAS domain of HIF-1α was found to interact with FoxA2 and the PAS domain of HIF-1α is known to interact with HIF-1β (Erbel et al., 2003), alternation of the FoxA2 level in vitro or in TRAMP-C cells, had no apparent effect on the interaction of HIF-1β with HIF-1α (FIGS. 3J and 3K).

vi. A Subset of HIF Target Genes is Cooperatively Regulated by FoxA2/HIF-1α.

Transcript levels of the HIF targets VEGFA and Glut-1 were not altered following FoxA2 expression or co-expression of HIF-1α and FoxA2 in TRAMP-C, PC3, and HeLa cells indicating that FoxA2/HIF-1α cooperation selectively affects HIF-regulated genes. Gene expression profiles of TRAMP-C cells transfected with pcDNA control vector, or expression vectors encoding FoxA2, HIF-1α, or HIF-1α plus FoxA2 were compared. Approximately 140 genes in TRAMP-C cells were upregulated by hypoxia (Table 2). Comparison of genes expressed in each condition identified 47 genes upregulated in the HIF-1α+FoxA2 group under hypoxia, in comparison with the other three groups (Table 4). Of these and other hypoxia-induced genes, 30 genes were further assessed for FoxA2-dependent expression, selected based on prostate- and NE tumor-specific expression. To confirm FoxA2-dependent transcription of this gene set, changes in their expression in TRAMP-C cells expressing FoxA2 siRNA were monitored. qRT-PCR analysis of selected genes identified Hes6, Sox9, Jmjd1a and Plod2 among those that displayed FoxA2-dependent transcription under hypoxia (FIG. 4A). Sox9, Jmjd1a and Plod2 are known HIF target genes (Amarilio et al., 2007; Beyer et al., 2008; Hofbauer et al., 2003). In contrast, transcription of Glut-1 and VEGFA was not altered following FoxA2 knockdown (FIG. 4A). Overall, these results indicate that in TRAMP-C cells FoxA2 cooperates with HIF-1α to regulate a subset of HIF targets under hypoxia.

TABLE 2
(related to FIG. 4) TRAMP-C cells transfected with HIF-1a and
FoxA2 were treated with 1% oxygen for 12 h.

| Gene Symbol | Genebank | Fold Increase |
|---|---|---|
| Cdkn1a | NM_007669.2 | 16.4 |
| Zbtb8 | NM_153541.1 | 11.9 |
| Adm | NM_009627.1 | 11.8 |
| Stc1 | NM_009285 | 11.5 |
| Galr2 | NM_010254.2 | 10.5 |
| Ccng2 | NM_007635.2 | 10.4 |
| Ndrl | NM_008681 | 10.2 |
| Ppp1r3b | NM_177741.2 | 9.9 |
| Plod2 | NM_011961 | 9.9 |
| Krt1-19 | NM_008471.1 | 9.2 |
| Ndrg1 | NM_010884.1 | 8.8 |
| Egln3 | NM_028133.1 | 8.8 |
| Hk1 | NM_010438 | 8.8 |
| Ppp1r3c | NM_016854.1 | 7.8 |
| Espn | NM_207690.2 | 7.1 |
| Cdc42ep2 | NM_026772 | 7 |
| Pfkp | NM_019703 | 6.8 |
| Ndrg2 | NM_013864 | 6.7 |
| Ak4 | NM_009647.1 | 6.7 |
| Wdt3 | XM_282971 | 6.5 |
| Nos3as | NM_001002897.2 | 5.9 |
| Egln1 | NM_053207.1 | 5.8 |
| Als2cr13 | NM_001037725.1 | 5.7 |
| Aldoc | NM_009657.2 | 5.6 |
| Gpr146 | NM_030258.2 | 5.4 |
| Pdk1 | NM_172665.1 | 5.3 |
| Triobp | NM_001039155.1 | 5.3 |
| Slc27a3 | XM_130954.3 | 5.2 |
| P4ha2 | NM_011031.1 | 4.9 |
| Ero1l | XM_122699.1 | 4.7 |
| Gm129 | NM_001033302.1 | 4.7 |
| Rora | NM_013646.1 | 4.7 |
| Vldlr | NM_013703.1 | 4.6 |
| Ampd3 | NM_009667 | 4.6 |
| Slc41a2 | NM_177388.2 | 4.5 |
| Rnf126 | NM_144528.1 | 4.5 |
| Slc2a1 | NM_011400.1 | 4.4 |
| Vegfa | NM_001025250.2 | 4.3 |
| Mxi1 | NM_010847.1 | 4.2 |
| Ddit4 | NM_029083.1 | 4.2 |
| Sox9 | NM_011448.2 | 4.1 |
| Pfkl | NM_008826.2 | 4.1 |
| Hig1 | NM_019814.2 | 4 |
| Lpin1 | NM_015763 | 4 |
| Jmjd1a | XM_194279.2 | 3.9 |
| Cryab | NM_009964.1 | 3.9 |
| Dixdc1 | NM_178118 | 3.9 |
| Jmjd2b | NM_172132.1 | 3.8 |
| Arrdc3 | NM_178917.2 | 3.8 |
| Pcyt1b | NM_211138.1 | 3.6 |
| Rhobtb1 | XM_125637 | 3.5 |
| Gls2 | NM_001033264.1 | 3.5 |
| Bhlhb2 | NM_011498.2 | 3.5 |
| Kcnb1 | NM_008420.3 | 3.4 |
| Hmox1 | NM_010442.1 | 3.4 |
| Mvd | NM_138656.1 | 3.4 |
| Sqle | NM_009270.2 | 3.4 |
| Abcb6 | NM_023732.2 | 3.4 |
| ler3 | NM_133662.1 | 3.4 |
| Plekha2 | NM_031257.2 | 3.3 |
| Nek1 | XM_356077.1 | 3.3 |
| Nat6 | NM_019750.2 | 3.3 |
| Map3k1 | NM_011945.2 | 3.2 |
| Sh3yl1 | NM_013709.2 | 3.2 |
| Pgf | NM_008827.2 | 3.2 |
| Fzd1 | NM_021457.2 | 3.1 |
| Gpr120 | NM_181748.1 | 3.1 |
| Gm22 | XM_111398.4 | 3.1 |
| Pcdh21 | NM_130878.2 | 3 |
| Col12a1 | NM_007730.2 | 3 |
| Nrn1 | NM_153529.1 | 3 |
| Lox | NM_010728.1 | 3 |
| Pdxp | NM_020271.1 | 3 |
| Car9 | NM_139305.1 | 2.9 |
| Centb1 | NM_153788.2 | 2.9 |
| Pkp2 | NM_026163.1 | 2.9 |
| Mpp2 | NM_016695.1 | 2.9 |
| Sertad 1 | NM_018820.3 | 2.9 |
| Spg21 | NM_138584.1 | 2.9 |
| Vhlh | NM_009507 | 2.8 |
| Mt2 | NM_008630.1 | 2.8 |
| Me2 | NM_145494 | 2.8 |
| Gtf2e2 | NM_026584.2 | 2.8 |
| Rnf19 | NM_013923.1 | 2.8 |
| Lss | NM_146006 | 2.8 |
| Cyp2s1 | NM_028775.2 | 2.8 |
| Ptdsr | NM_033398.1 | 2.8 |
| Igfbp3 | NM_008343 | 2.7 |
| Gpi1 | NM_008155 | 2.7 |
| Grhpr | NM_080289.1 | 2.7 |
| CRG-L1 | NM_139306.1 | 2.7 |
| Reep1 | NM_178608.2 | 2.7 |
| Plod 1 | NM_011122.1 | 2.6 |
| Homer1 | NM_152134.1 | 2.6 |
| Cd109 | NM_153098.2 | 2.6 |
| Nsdhl | NM_010941.3 | 2.6 |
| Dyrk1b | NM_001037957.1 | 2.6 |
| Ostf1 | NM_017375.1 | 2.6 |
| Efna 1 | NM_010107.2 | 2.6 |
| Tnfsf9 | NM_009404.1 | 2.6 |
| Hk2 | NM_013820.1 | 2.6 |
| Cyp51 | NM_020010 | 2.6 |
| Ctns | NM_031251.2 | 2.5 |
| Slc19a2 | NM_054087.1 | 2.5 |
| Itpk1 | NM_172584.1 | 2.5 |
| Dusp1 | NM_013642.1 | 2.5 |
| Trerf1 | NM_172622.1 | 2.5 |
| Irx2 | NM_010574.2 | 2.5 |
| Hpse | NM_152803.3 | 2.4 |
| RasI12 | NM_001033158.1 | 2.4 |
| Bnip3 | NM_009760.2 | 2.4 |
| Hes6 | NM_019479.2 | 2.4 |
| Mboat2 | NM_026037.2 | 2.4 |
| Capn5 | NM_007602.2 | 2.4 |
| Sap30 | NM_021788.1 | 2.4 |
| Acat2 | NM_009338.1 | 2.4 |
| Foxo3 | NM_019740 | 2.4 |
| Gbe 1 | AK050423 | 2.4 |
| Whdc1 | NM_001004185.2 | 2.4 |
| Pgm2 | NM_028132.2 | 2.3 |
| P4ha1 | NM_011030.1 | 2.3 |
| Elmo1 | NM_198093.2 | 2.3 |
| Insig1 | NM_153526.2 | 2.3 |
| Kif21b | NM_019962.2 | 2.3 |
| Aldoa | NM_007438.2 | 2.3 |
| Hdac5 | NM_001077696.1 | 2.3 |
| Bcl2l11 | NM_207680.1 | 2.3 |
| Acas2 | NM_019811.2 | 2.2 |
| Scd2 | NM_009128.1 | 2.2 |
| Casp6 | NM_009811.2 | 2.2 |
| Siah2 | NM_009174.2 | 2.2 |
| Pgam1 | NM_023418.2 | 2.2 |
| Pafah1b3 | NM_008776.1 | 2.2 |
| Hoxa11s | XM_149724.5 | 2.2 |
| Hmgcl | NM_008254.1 | 2.2 |
| Il13ra1 | NM_133990.3 | 2.2 |
| Kdelr3 | NM_134090 | 2.1 |
| Gpr35 | NM_022320.2 | 2.1 |
| Itga11 | NM_176922.4 | 2.1 |
| Gch1 | NM_008102.2 | 2 |
| Adipor2 | NM_197985.2 | 2 |
| Kcnk2 | NM_010607.1 | 2 |

RNA was isolated for microarray analysis as detailed in Materials and Methods.
Gene expression profiles under hypoxia and normoxia were compared, and genes that are >2-fold upregulated are shown.
Of these, 30 genes were selected for further characterization based on expression levels in prostate tumors.

TABLE 3
Hypoxia-downregulated genes in TRAMP-C cells
co-transfected with HIF-1α and FoxA2 (hypoxia vs normoxia).

| Gene Symbol | Genebank | Fold Increase |
|---|---|---|
| Pigt | NM_133779 | 4.7 |
| Cyr61 | NM_010516.1 | 3.3 |
| Rapgef6 | NM_175258.3 | 3.2 |
| Gemin4 | NM_177367.2 | 3.2 |
| Csf1 | NM_007778.1 | 3.2 |
| Ftsj3 | NM_025310.2 | 3.2 |
| Hist1h1c | NM_015786 | 3.2 |
| Sdpr | NM_138741.1 | 3.1 |
| Rasl11a | NM_026864.1 | 3.1 |
| Mat2a | NM_145569 | 3 |
| Gpr73 | NM_021381.3 | 3 |
| Brp16 | NM_021555.1 | 3 |
| Slc11a2 | AK049856 | 2.8 |
| Cd44 | AK045226 | 2.8 |
| Ankrd1 | NM_013468.2 | 2.8 |
| Ccl2 | NM_011333.1 | 2.8 |
| Ccl7 | NM_013654.2 | 2.8 |
| Fjx1 | NM_010218 | 2.7 |
| Chchd4 | NM_133928.1 | 2.7 |
| Tgm2 | NM_009373.2 | 2.6 |
| Fpgs | NM_010236.1 | 2.5 |
| Sprr2h | XM_147310.1 | 2.5 |
| Timm13a | NM_013899.1 | 2.5 |
| Rpo1-4 | AK031689 | 2.4 |
| Ddx21 | AK012125 | 2.3 |
| Ase1 | NM_145822.1 | 2.3 |
| Socs2 | NM_007706.1 | 2.3 |
| Pinx1 | NM_028228.1 | 2.3 |
| Qtrtd 1 | XM_148491.1 | 2.3 |
| Ddx4 | NM_010029.1 | 2.3 |
| Gbif | NM_019683.2 | 2.3 |
| Egr1 | NM_007913.2 | 2.3 |
| Arv1 | NM_026855.1 | 2.3 |
| Rock2 | NM_009072.1 | 2.2 |
| Ccnd1 | NM_007631.1 | 2.2 |
| Usp1 | NM_146144.2 | 2.2 |
| Dbt | NM_010022.1 | 2.2 |
| Twistnb | NM_172253.1 | 2.2 |
| Pprc1 | XM_359412.1 | 2.2 |
| Mki67ip | NM_026472 | 2.2 |
| Setd6 | NM_001035123.1 | 2.2 |
| Dp1l1 | NM_139292.1 | 2.1 |
| Tdrkh | XM_131021.5 | 2.1 |
| Zdhhc21 | NM_026647.2 | 2.1 |
| Hspbap1 | NM_175111 | 2.1 |
| Rpo1-2 | NM_009086.1 | 2 |
| Speer6-ps1 | NR_001581.1 | 2 |

TRAMP-C cells transfected with HIF-1a and FoxA2 were treated with 1% oxygen for 12 h.
RNA was isolated for microarray analysis as detailed in Materials and Methods.
Gene expression profiles under hypoxia and normoxia were compared, and genes that are >2-fold downregulated are shown.

TABLE 4
(related to FIG. 4) Upregulated genes in TRAMP-C cells
co-transfected with HIF-1α and FoxA2 in comparison with
those transfected with pcDNA, HIF-1α, or FoxA2 alone.

| Gene Symbol | Genebank | Fold Increase |
|---|---|---|
| Itga5 | NM_010577.2 | 3.3 |
| Sepw1 | NM_009156.1 | 2.9 |
| Mpp2 | NM_016695.1 | 2.7 |
| Zfp57 | NM_009559.2 | 2.7 |
| Ihpk2 | NM_029634.1 | 2.6 |
| Ppm1g | NM_008014.2 | 2.6 |
| Prss35 | NM_178738.1 | 2.5 |
| Cnot3 | NM_146176.1 | 2.4 |
| Rai3 | NM_181444 | 2.4 |
| Hk1 | NM_010438 | 2.3 |
| Lbh | NM_029999.3 | 2.3 |
| Fn1 | XM_129845.3 | 2.3 |
| Drpla | NM_007881.3 | 2.3 |
| Aes | NM_010347 | 2.2 |
| F2rl1 | NM_007974.2 | 2.2 |
| Mark2 | NM_007928.2 | 2.2 |
| Actn4 | NM_021895.2 | 2.2 |
| Fscn1 | NM_007984.1 | 2.2 |
| Catns | NM_007615 | 2.2 |
| Lsm2 | NM_030597.2 | 2.2 |
| Srm | NM_009272.2 | 2.2 |
| Cited2 | NM_010828 | 2.1 |
| Pcqap | NM_033609.1 | 2.1 |
| Fath | XM_134149 | 2.1 |
| Trpc4ap | NM_019828.1 | 2.1 |
| Carm1 | NM_021531.1 | 2.1 |
| Slc16a3 | NM_001038653.1 | 2.1 |
| Dusp10 | NM_022019.2 | 2.1 |
| Ngfb | NM_013609.1 | 2.1 |
| Jund1 | NM_010592.3 | 2.1 |
| Actn4 | NM_021895.2 | 2.1 |
| Jmjd1a | NM_173001.1 | 2 |
| Rnf126 | NM_144528.1 | 2 |
| Mybbp1a | NM_016776 | 2 |
| Plod2 | NM_011961.1 | 2 |
| Bcl2l1 | NM_009743.2 | 2 |
| Abcf2 | NM_013853.1 | 2 |
| Co16a2 | NM_146007.1 | 2 |

Transfected TRAMP-C cells were maintained in 1% oxygen for 12, and RNA was isolated for microarray analysis.
The gene expression profile of TRAMP-C cells co-transfected with HIF-1α and FoxA2 was compared with those transfected with pcDNA control, HIF-1α or FoxA2 alone.
Genes upregulated >2-fold are shown.

vii. HIF-1α and FoxA2 Cooperate to Activate Hes6 Transcription.

The transcription factor Hes6 is reportedly highly upregulated in human metastatic prostate cancers with a NE phenotype (Vias et al., 2008). Upregulation of Hes6 transcripts under hypoxia indicates that Hes6 is a HIF target gene. Indeed, knockdown of HIF-1α or HIF-1β reduced hypoxia-induced Hes6 transcription (FIG. 4B). Three potential HREs were identified and the corresponding 1.25 kb of the mouse Hes6 promoter region upstream of a luciferase reporter was cloned. This construct was activated 2.5 fold by the hypoxia mimic DMOG (FIG. 10A) and 1.8 fold by hypoxia. Deletion analysis of the Hes6 promoter revealed that the HRE at −66 bp was required for hypoxia-induced reporter activity (FIG. 10A). Mutation of this HRE in the full-length 1.25 kb fragment resulted in loss of the response to DMOG (FIG. 4C). FoxA2 knockdown repressed Hes6 transcription (FIGS. 4A, 4B), while FoxA2 overexpression increased Hes6 promoter activity following addition of DMOG, an effect not seen using the HRE-mutant promoter construct (FIG. 4D). These observations indicate that FoxA2-induced Hes6 promoter activity requires an HRE and thus HIF activity. Chromatin immunoprecipitation (ChIP) confirmed that HIF-1α and HIF-1β bound the −66 bp HRE but not the other two putative HREs in the HES6 promoter under hypoxia (FIG. 10B). ChIP analysis confirmed the binding of FoxA2 to the same −66 bp HRE, which was impaired following HIF-1α knockdown (FIG. 10C), indicating that FoxA2 is recruited to the promoter through HIF-1α. Similarly, FoxA2 bound to HRE-containing promoter regions of Sox9 (FIG. 10C) and Jmdj1a in a HIF-1α-dependent manner. These results indicate that FoxA2 regulates a subset of HIF target genes possibly through direct binding to HIF.

viii. FoxA2-Dependent Tecruitment of p300 to the Promoters of HIF Target Genes.

To analyze mechanisms underlying selectivity of FoxA2 cooperation with HIF-1α, whether FoxA2 expression changed HIF-1α levels, its asparagine hydroxylation, or its binding to the Hes6 promoter was investigated but none were found. To directly assess the contribution of FoxA binding sites to FoxA2/HIF-1α cooperation, possible changes in FoxA2/HIF-1α-mediated transactivation was determined using Hes6 promoter mutants (FIG. 10D). While a single FoxA site was sufficient to retain full responsiveness to FoxA2/HIF-1α cooperation, a mutation within this element attenuated such cooperation (FIG. 10D). Notably, analysis of the effect of FoxA2 on recruitment of p300, a co-activator of HIF transcriptional activity (Arany et al., 1996), revealed that binding of p300 to the Hes6 promoter was attenuated following FoxA2 knockdown (FIG. 4E). FoxA2 knockdown also reduced the binding of p300 to HRE-containing promoters of Jmjd1a and VEGFA in both TRAMP-C cells and Rv1 cells (FIG. 4F). However, p300 knockdown only reduced the transcript levels of Hes6 and Jmjdla but not VEGFA (FIG. 4G), which resembled changes seen upon knockdown of FoxA2 (FIG. 4A) on the hypoxia-induced transcription of these genes. These results indicate that p300 serves a distinct FoxA2-dependent HIF transcriptional program. Consistent with this possibility, HIF-induced promoter activity of Hes6 was more sensitive to increased p300 levels than that of VEGFA (FIG. 4H). Notably, p300, but not p300ΔCH1 (a p300 mutant that cannot interact with HIF-1α), could further increase the degree of HIF/FoxA2 effect on Hes6 promoter activity (FIG. 4I). In contrast, FoxA2 and p300 showed no apparent effect on HIF-dependent activation of the VEGFA promoter (FIG. 4J). These results indicate that recruitment of p300 is, in part, responsible for the degree and the selectivity of the transcriptional program elicited by HIF/FoxA2 cooperation.

In vitro protein binding showed that FoxA2 enhanced HIF-1α/p300 interaction but had no effect on p300 binding to the HIF-1α 531-822 mutant that cannot associate with FoxA2 (FIGS. 9C, 3H, 4K). p300 lacking the CH1 domain neither interacted with the HIF/FoxA2 complex (FIG. 4L) nor enhanced Hes6 transcription by HIF/FoxA2 (FIG. 4I). These results indicate that FoxA2 promotes HIF and p300 interaction unlikely require HIF-1α N-TAD and p300 CH3 domain (Ruas et al., 2010).

ix. Genes Regulated by HIF/FoxA2 are Important for Tumorigenesis of TRAMP-C Cells.

To determine whether genes regulated by HIF/FoxA2 cooperation are important for tumorigenesis retroviral vectors were employed to re-express Hes6, Sox9 and Jmjd1a individually or in combination in TRAMP-C cells stably expressing PHYL or FoxA2 shRNA. While the endogenous expression of these genes was attenuated upon expression of PHYL peptide or FoxA2 shRNA, their transcript levels were restored following ectopic expression (FIGS. 11A, 11B). TRAMP-C cells expressing control (pBabe vector) or N×N, a mutant PHYL that cannot interact with Siah, but not those expressing PHYL or shFoxA2 could form colonies on soft agar (FIGS. 5A, 5B). Significantly, co-expression of Hes6, Sox9 and Jmjd1a effectively rescued the ability of TRAMP-C cells expressing either PHYL or shFoxA2 to form colonies in soft agar but re-expression of each individually was insufficient (FIG. 5A, 5B). Consistent with the effect of PHYL being Siah-dependent, reduction of Siah1a and Siah2 (FIG. 11C) reduced levels of HIF-1α protein (FIG. 11D) and Hes6, Sox9 and Jmjd1a transcripts (FIG. 11E), and attenuated the ability of these cells to form colonies in soft agar under hypoxia (FIG. 11F). The reduced ability to form colonies in soft agar could be partially rescued upon re-expression of Hes6, Sox9 and Jmjd1a (FIG. 11F).

Similar to the findings in culture, control but not the PHYL-expressing TRAMP-C cells were able to form tumors upon injection into the prostate of mice (FIGS. 5D, 5E). Whereas re-expression of Hes6, Sox9 or Jmjd1a individually in PHYL-expressing TRAMP-C cells failed to rescue tumorigenesis, expression of all three was able to restore (4/5 mice) tumorigenicity and partially rescue tumor growth (30% of tumor size) (FIGS. 5D, 5E). Orthotopic injection of shFoxA2-expressing TRAMP-C cells into the prostate also showed significant reduction in tumor formation, which was almost fully rescued by co-expression of Hes6, Sox9 and Jmjd1a (FIG. 5F, 5C). These results further establish the importance of HIF/FoxA2 target genes, Hes6, Sox9 and Jmjd1a, in the development of NE prostate tumor.

x. Genes Co-Regulated by HIF and FoxA2 are Important for Hypoxia-Associated NE Phenotype and Metastasis of Human Prostate Adenocarcinoma Cells.

The importance of the pathway discovered using the TRAMP model in human Pca was assessed next. To this end CWR22Rv1 cells (Rv1) were selected, which were derived from a human prostate adenocarcinoma xenograft displaying an NE phenotype (Huss et al., 2004; Sramkoski et al., 1999). Rv1 cells grown under hypoxia showed upregulation of NE markers NSE and chromogranin B (ChgB) (FIGS. 6A, 6B) and demonstrated protrusion of neurite-like structures from cells located at the periphery of colonies. Concomitant with the induction of NE phenotype was the upregulation of Hes6, Sox9 and Jmjd1a transcripts (FIG. 6D). Significantly, inhibition of Siah or knockdown of FoxA2 attenuated hypoxia-induced upregulation of Hes6, Sox9 and Jmjd1a (FIGS. 12A, 12B). To determine whether Hes6, Sox9 and Jmjd1a are important for hypoxia-induced NE phenotype, these genes were re-expressed individually or in combination in PHYL- or shFoxA-expressing Rv1 cells (FIG. 12A, 12B) and only co-expression of all three could partially restore hypoxia-induced NSE upregulation and formation of neurite-like structures (FIGS. 6E, 6F, 6G). These results point to a requirement for Siah-HIF/FoxA2 regulated genes in the hypoxia-induced NE phenotype of human prostate adenocarcinoma cells.

To evaluate the biological significance of NE phenotype for human prostate cancer in vivo, the above Rv1 transfectants were injected into the prostate of nude mice. Surprisingly, unlike TRAMP-C cells, Rv1 cells expressing PHYL only showed about 30% reduction in the tumor size which could not be rescued by co-expression of Hes6, Sox9 and Jmjd1a (FIG. 6H). Similarly, Rv1 cells expressing shFoxA2 did not exhibit any notable decrease in tumor formation (FIG. 6H). These results indicate that Hes6, Sox9 and Jmjd1a are not involved in tumorigenesis of Rv1 cells. PHYL expression reduces the vessel density of Rvl allograft tumor. The 30% reduction of tumor size by PHYL was correlated with a 35% reduction in the tumor vessel density (Table 5) and 40% reduction of VEGFA transcript in PHYL-expressing Rv1 cells in vitro (FIG. 12D). Similarly, colony formation in the soft agar assay was comparable for PHYL-expressing Rvl cells and control cells (FIG. 12C). Significantly, the circulating PHYL- or shFoxA2-expressing Rv1 cells in the blood were reduced, indicating impaired intravasation of these cells, which was partially rescued by co-expression of Hes6, Sox9 and Jmjd1a (FIG. 6I). Although Rv1 cells exhibited limited ability to form liver metastasis (2 out of 10 mice injected with Rv1 cells), they were very efficient in periaortic lymph node metastases. Expression of PHYL or shFoxA2 abolished lymph node metastases of Rvl cells, which could be largely restored by co-expression of Hes6, Sox9 and Jmjd1a (FIG. 6C). These findings indicate that genes co-regulated by HIF and FoxA2 play a key role in metastasis of prostate adenocarcinoma cells.

TABLE 5
(related to FIG. 6) Quantification of PCNA,
active caspase-3 and CD31 in Rv1 allograft.

| | PCNA (%) | Caspase-3 (%) | CD31 (#/field) |
|---|---|---|---|
| pBabe | 91.0 ± 1.0 | 1.2 ± 1.0 | 19.5 ± 5.1 |
| PHYL | 89.0 ± 5.1 | 1.3 ± 1.2 | 13.2 ± 4.1 |
| PHYL + HSJ | 89.6 ± 7.1 | 1.3 ± 1.1 | 13.0 ± 3.1 |
| shFoxA2 | 89.4 ± 4.7 | 1.7 ± 1.3 | 20.0 ± 4.0 |
| shFoxA2 + | 88.4 ± 4.1 | 1.5 ± 1.1 | 18.6 ± 4.9 |
| HSJ | | | |
| p value | p > 0.1 between | p > 0.1 between | P < 0.01 (pBabe vs. |
| | any two groups | any two groups | PHYL) |
| | | | P < 0.05 pBabe vs. |
| | | | PHYL + HSJ |

Allograft tumor sections formed by intraprostatic injection of Rv1 transfectants indicated were subjected to IHC staining of PCNA, active caspase-3 and CD31.
The percentage of PNCA (+) or active caspase-3 (+) nucleus was calculated using the nuclear quantification algorism of Scanscope on 10 random high-power fields.
The # of CD31(+) vessels was manually counted on 10 random high-power fields.

IHC analyses of Rv1 allograft tumors revealed that HIF-1α and NSE staining were concentrated around the necrotic regions, which are known to be highly hypoxic, whereas FoxA2 staining was evenly distributed. As expected, expression of PHYL resulted in reduced HIF-1α staining and loss of NSE staining in the hypoxic regions. FoxA2 shRNA reduced the NSE staining in the hypoxic regions without affecting HIF-1α levels, consistent with the in vitro data (FIGS. 6E, 6F, 6G). Importantly, consistent with the in vitro results, co-expression of Hes6, Sox9 and Jmjd1a in PHYL- or shFoxA2-expressing Rvl cells restored the NSE staining in the hypoxic regions (FIGS. 6A, 6B, 6D, 6E, 6F, and 6G). Strong NSE staining was primarily seen within the more hypoxic regions, proximal to the necrosis, of the primary tumor and metastatic lesions in liver and lymph nodes, implying that the NE-differentiated Rv1 cells is responsible for the metastasis. These results establish that genes co-regulated by HIF and FoxA2 play a key role in hypoxia-induced NE phenotype of PCa in vitro and in vivo, and that NE phenotype is tightly associated with PCa metastasis.

xi. Expression of FoxA2-HIF-1α Target Genes in Prostate Tumors.

The issue of whether FoxA2/HIF-1α transcriptional targets were expressed in NE tumors was addressed. The very few NE tumors identified in a TRAMP/Siah2^{-/-} mice had lower transcript levels of Hes6, Sox9, Jmjd1a and Plod2 compared with TRAMP/Siah2^{-/-}-derived NE tumors (FIG. 7A), consistent with HIF-1α-dependent expression.

The NE phenotype seen in human PCa can be classified to three types based on IHC staining of NE markers (Cindolo et al., 2007; Hirano et al., 2005; Shimizu et al., 2007). Focal—where NE markers distinguish clusters of cells—found in low-grade and moderately differentiated PCa; general staining—where larger tumor areas are positive for NE markers—found in high-grade and poorly differentiated PCa; and single cells that are stained positively for NE markers (Hirano et al., 2005). Fifteen cases of human Pca were examined and 10 were found to exhibit NE phenotype. Two of the 10 samples displayed focal staining of NE marker NSE, where FoxA2, Hes6 and Sox9 were concentrated in the NE foci. Eight of the 10 specimens showed a general staining of NSE, with co-staining of FoxA2, Hes6 and Sox9 in 5/8 cases. Co-expression of FoxA2, Hes6 and Sox9 found in 70% of PCa specimens with NE phenotype was statistically significant (p<0.05; FIG. 7C). Expression of FoxA2, Hes6, Sox9 and HIF1-a was examined in Prostatic Intraepithelial Neoplasia (PIN) with NED foci. The PIN is characterized by solid growth with expansion of the glandular lumen and central cellular necrosis, partially involving the gland space. The neuroendocrine differentiation is confirmed by the staining of synaptophysin and NSE, which highlight nest of NE PIN cells undermining the adjacent normal epithelium in a “pagetoid” spread. The same cells can also be seen positive for the FoxA2, Sox9 and HIF-1α stains. Hes6 appears to stain a smaller subset of these NE PIN cells.

IHC staining of a human prostate ™ A was performed consisting of 79 cases representing prostatic intraepithelial neoplasia (PIN) and different Gleason stages of PCa. A higher NSE staining was found in high-grade PCa (G4 and G5), which also showed increased staining of Siah2, FoxA2, Hes6 and Sox9, compared with low-grade tumors (PIN and G3) (FIG. 7B). The difference in the expression of NSE, Siah2, FoxA2, Hes6 and Sox9 between high-grade and low-grade tumors is statistically significant and correlated with pathoclinical staging. In addition, IHC staining of 3 human prostate NE tumors revealed co-expression of synaptophysin, FoxA2, HIF-1α, Hes6 and Sox9 in all cases. These findings indicate that NE-positive tumors are associated with the more malignant human prostate cancers, in which the HIF/FoxA2 target genes are expected to play a role in the development of the NE phenotype. In agreement, gene expression data from human prostate adenocarcinoma identified a marked increase in Hes6, Plod2, Jmjd1a and FoxA2 expression in metastatic prostate tumors, compared with primary prostate tumors and normal prostate tissues (FIG. 7D). The expression of these genes correlates with the expression of NE markers, further illustrating the link between their expression, NED and metastatic prostate tumors (FIG. 7D).

Whereas Siah2 staining was higher in high-grade PCa, HIF-1α staining was found in both low-grade and high-grade tumors (FIG. 7B). Despite high level of HIF-1α expression in all grades of tumors, the level of Glut-1, a common readout for HIF transcriptional activity, was high only in high-grade PCa, pointing to a correlation between Siah2 expression and HIF-1α activity (FIG. 7B). In agreement, gene expression analyses revealed an increased Siah2 transcript and enhanced HIF activity in metastatic PCa as reflected by increased transcript of HIF target genes such as CA9, VEGFA and Glut-1, compared with primary PCa (FIG. 7D). These results substantiate the correlation between Siah2 expression and HIF activity in human PCa, consistent with the role of Siah in regulation of PHD3 and factor-inhibiting HIF-1 (FIH) stability, which control HIF-1α availability and activity (Nakayama et al., 2004; Fukuba et al., 2008). The cooperation between HIF and FoxA2 in determining NE phenotype can be attributed to a higher level of Siah2 which increases HIF stability and activity, and availability of FoxA2 in the high-grade PCa.

2. Discussion

The results provide insight into regulation and function of the FoxA2/HIF-1α complex in determining NE prostate tumor formation and NE phenotype, an important component of metastatic prostate adenocarcinomas. These results also point to a role for Siah2 in determining tumor differentiation. Siah2 loss has little effect on development and growth of the prostate luminal epithelium but decreases initiation of NE carcinomas and, consequently, the metastatic burden in the TRAMP model. Partial deletion of Siah1a on a Siah2-null background fully ablated NE tumor formation, indicating that both Siah2 and Siah1 are required to enable the development of prostate NE tumors.

As HIF-1α is stabilized under hypoxia and FoxA2 is expressed in NE tissues, these findings indicate conditional and spatial cooperation between these two factors under specific tissue and oxygen requirements. Siah2-dependent regulation of HIF coupled with NE-specific expression of FoxA2 provides a framework for a specific tumor differentiation program associated with a highly metastatic phenotype. Among the four FoxA2/HIF targets identified in this study, Hes6 is reportedly highly upregulated in metastatic prostate cancers displaying NE markers (Vias et al., 2008) and Plod2 is overexpressed in metastatic prostate NE tumors (Shah et al., 2004). Sox9 expression is increased in relapsed androgen-refractory prostate cancers and is associated with enhanced growth, invasion and angiogenesis (Wang et al., 2008). It is noteworthy that Hes6, Sox9 and Jmjd1a have been shown to regulate differentiation of stem/progenitor cells (Eun et al., 2008; Loh et al., 2007; Nowak et al., 2008), indicating that FoxA2/HIF cooperation initiates a transcriptional program that regulates neuroendocrine differentiation of normal and/or prostate cancer stem cells. In agreement, the NE phenotype of PCa is also associated with expression of the stem/progenitor markers, supporting the notion that the NE-like cells harbor prostate cancer stem cells (Bonkhoff, 1998; Helpap et al., 1999; Sotomayor et al., 2008).

Several plausible mechanisms could underlie HIF-1α/FoxA2 transcriptional synergy, the intrinsic chromatin remodeling activity of FoxA2 being important (Cirillo et al., 2002) and that FoxA2 displaces HIF-1β were ruled out. The data implicate FoxA2 in enhanced recruitment of p300 for the selective activation of a subset of HIF-target genes. Consistent with these findings, MEFs from mice expressing a p300/CBP mutant that cannot interact with HIF exhibited attenuated HIF activity in luciferase reporter assays but they showed attenuated expression of only a small subset of HIF target genes, not including VEGFA and Glut-1 (Kasper et al., 2005).

The importance of Hes6, Sox9 and Jmjd1a for NE phenotype is demonstrated in human PCa cells that exhibit NE phenotype under hypoxia. Inhibition of these genes attenuates the NE phenotype and PCa metastasis, whereas their co-expression rescues the NE phenotype and metastasis even upon knockdown of FoxA2 or Siah2—their upstream regulators. Notably, the requirement of hypoxia for NE phenotype was confirmed by IHC analyses of PCa in vivo where level of NSE coincided with that of HIF, FoxA2 and their regulated genes. Of equal significance, inhibition of mouse prostate tumor growth by attenuating Siah2 activity could be overcome upon co-expression of Hes6, Sox9 and Jmjd1a, further illustrating the importance of their cooperation for prostate tumor development.

Overall, the study offers a paradigm underlying formation of NE phenotype and NE prostate tumor development. This pathway requires the ubiquitin ligase Siah2, which determines the level and activity of HIF-1α, which then cooperates with the NE-specific transcription factor FoxA2. It is the conditional and spatial regulation of these factors that transactivates a subset of genes critical for NE phenotype and metastasis of PCa as well as for development of NE prostate tumors. Common to NE prostate tumors and PCa harboring the NE phenotype is their strong propensity to metastasize, and the poor outcome associated with these more aggressive forms of prostate cancer. These findings unveil mechanisms underlying their development and progression, respectively, while identifying targets for therapy and markers for improved detection and monitoring of these tumors.

3. Experimental Procedures

Prostate tumor samples. Prostate tumor samples representing NE tumors and/or prostate adenocarcinomas were obtained as part of approved clinical studies from the University of California Davis (IRB #200312072), University of California Irvine (SPECS project, IRB #20005-4806) and Northwestern University (SPORE tissue banking protocol, IRB #NCI01X2: STU00009126). In all cases informed consent was obtained from all subjects.

Animal studies. All animals were housed in the Sanford-Burnham Institute's animal facility and the experiments with live animals were approved by the institute's animal committee (IACUC #04-135, 04-141, 07-132) and conducted following the Institute's animal policy in accordance with NIH guidelines.

Cell Lines. TRAMP-C cells were maintained in Dulbecco's modified Eagle's medium (DMEM) supplemented with 5% Nu-serum IV, 5% fetal bovine serum (FBS), 5 μg/ml insulin and antibiotics. CWR22 Rv1 cells were maintained in RPMI1640 medium with 5% FBS and antibiotics.

Generation of TRAMP mice in a Siah2 knockout background. Siah2^{-/-} mice (129 SVJ strain) were crossed with TRAMP transgenic mice (C57/B16 strain) to obtain Siah2 heterozygotes carrying the TRAMP transgene (C57/B16 and 129 SVJ mixed strain). Female Siah2^{+/-}/TRAMP mice were crossed with male Siah2+/− mice to generate male TRAMP mice of three genotypes (Siah2^{+/+, +/-, -/-}), which were predominantly of the 129 strain. Female Siah2^{+/-}/TRAMP mice were also crossed with male Siah1a^{+/-}Siah2^{+/-} mice to generate male TRAMP mice with a Siah1a^{+/-}Siah2^{-/-} genotype. Siah/TRAMP mice were analyzed at 8 months of age.

Antibodies and reagents. Antibodies to HIF-1α, HIF-2α and Sox9 (NOVUS), to Hes6, Jmjd1a, p300 and NSE (Abcam), to synaptophysin (BD Bioscience), to FoxA2, HIF-1β and CD31, Chromogranin B (Santa Cruz), to active caspase-3 (Chemicon), to PCNA (Cell Signaling), to FLAG, HA, α-tubulin and β-actin (Sigma) were used according to manufacturers' recommendations. An ApopTag peroxidase in situ apoptosis kit was obtained from Chemicon.

Statistical analysis. Student's t-test or Fisher's exact test was used for the statistical analyses.

Accession number. Microarray data was deposited in the GEO database (GSE18478).

Plasmids, cloning and mutagenesis. Flag-tagged mouse Siah2 RING mutant in pcDNA3 vector, HA-tagged PHYL peptide in pKH3 vector, mouse HIF-1α and IPAS in pCVM-Flag vectors, and pT81-HRE-Luc constructs have been described (Qi et al., 2008). HA-PHYL peptide and HA-NXN peptide (mutant PHYL that does not bind Siah) were subcloned into pBabe puro vector. Mouse HIF-1α and HIF-1α 531-822 were subcloned into T vector for in vitro translation of proteins without Tag. Flag-HIF-1α truncation mutants in T vector were obtained from Dr. Lorenz Poellinger (Karolinska Institute, Stockholm, Sweden) and used for in vitro protein translation. HA-tagged rat FoxA1, FoxA2, and 6XFOXA-Luc construct in pcDNA3 vectors have also been described (Kim et al., 2004). Mouse FoxA2 sequence was obtained by RT-PCR, and cloned into pcDNA3 vector with a Flag-tag or a Myc-tag. FoxA2 truncation mutants were generated by PCR, and cloned into pcDNA3 vector with a Myc-tag. Mouse FoxA2 was subcloned into pET15b vector for expression and purification of His-tagged fusion protein. Flag-p300 construct was provided by Dr. Wei Gu (Columbia University, New York). p300 A CH1 (deletion of aa 326-379 of human p300) was generated by PCR subcloning, according to a published report (Kasper et al., 2005). Mouse HIF-1β sequence was purchased from Open Biosystem. Mouse and human Hes6, Sox9 and Jmjd1a sequences were purchased from Open Biosystem, and subcloned into pBabe hygro vector. Mouse Siah2 shRNAs, mouse Siah1a shRNAs, mouse and human FoxA2 shRNAs in pKLO.1 vector were purchased from Open Biosystems. The mouse 1.25-kb Hes6 and 1-kb VEGFA promoters were obtained by PCR using genomic DNA as template, and cloned into pGL-3b vector. Mutagenesis of the HRE or FOXA site on the Hes6 promoter was performed using QuickChange II XL mutagenesis kit (Invitrogen).

Immunohistochemistry. Sections prepared from tumors, organs or human prostate ™ A were rehydrated and processed for immunohistochemistry. Antigen retrieval was performed using Dako target retrieval solution, followed by peroxidase block for 30 min with 3% hydrogen peroxide. Specimens were incubated with primary antibody diluted in Dako antibody diluent overnight at 4° C. After incubation, slides were washed three times with PBS/Tween-20 and incubated with Dako Labelled Polymer-HRP (anti-rabbit, anti-mouse) for 1 h at RT. For the goat primary antibody, slides were incubated with Biotinylated donkey anti-goat secondary antibody followed by incubation with streptavidin-HRP, each for 1 h at RT. Slides were then washed 4 times with PBS/Tween-20, developed with DAB, and counterstained with hematoxylin. To quantify CD31 reactivity, the number of CD31-positive blood vessels was counted in 10 high-power fields. The results were presented as an average of CD31-positive vessels per high-power field. For PCNA or active caspase-3 staining, the percentage of positively stained nuclei was calculated in 10 high-power fields using the Scanscope algorithm for nuclear staining quantification. For TUNEL staining, the number of TUNEL-positive nuclei was counted in 10 high-power fields. IHC staining performed in the absence of primary antibodies was used as negative controls. For quantification of prostate TMA stained for HIF-1α, Glut-1, FoxA2, Hes6, Sox9 and NSE, the intensity of staining was divided into 4 scales: 0 (no staining), 1 (weak staining), 2 (medium staining) and 3 (strong staining) ™ A cores with staining scale of 2 and 3 were scored as positive, whereas those with staining scale of 0 and 1 were scored as negative.

Immunofluorescence Staining. Rv1 cells were grown on the 6-well plate under 1% O₂ for 6 days. Cells were fixed with 3.5% paraformaldehyde in PBS for 15 min, permeabilized with 0.1% Triton X-100 for 2 min, and blocked in 1% BSA for 1 h. Cells were incubated with NSE or ChgB rabbit polyclonal antibody (1:300) for 1 h, washed, and then incubated with Alexa 488 goat-anti-rabbit IgG (1:500) for 1 h. After washing, cells were incubated with 0.5 μg/ml of DAPI solution for 15 min to counterstain nuclei. Images were taken under a Leica fluorescence microscope.

Quantification of metastases. Livers were examined for visible surface tumors and then fixed using the Z-fix solution. Livers were grossly sectioned (˜1 mm thick) using a razor to determine whether there were any tumors present in the interior that were not visible when examining the liver surface. The fixed livers were embedded in paraffin blocks, and sliced to enable analyses of at least 5 serial sections per liver lobe. Similarly, fixed and embedded lung tissues were also sliced to obtain 5 serial sections per lung. Sections were stained with H&E to quantify the number and size of metastases under a histology microscope. All sections were analyzed for possible metastatic lesions.

Xenograft of TRAMP-C cells. TRAMP-C cells that stably express PHYL and HIF-1α were generated by transfecting specified vectors followed by selection with G418 (2 mg/ml) and puromycin (1 μg/ml). Expression of the ectopic proteins in these clones was verified with Western blotting. The stable transfectants (3×10⁶ cells/100 μl of 1:1 PBS and Matrigel) were injected subcutaneously to the flank of 8-week-old male nude mice. Tumor growth was monitored weekly for up to 8 weeks. Tumors were dissected and their size measured. Tumor volume was calculated by length×width×thickness.

Allograft of TRAMP-C or Rv1 cells. Intraprostatic injection was performed by the members of the animal facility at Sanford-Burnham Medical Research Institute. Briefly, 8-week-old male nude mice were prepared for surgery with anesthesia using Avertin (0.6 cc/30 g, i.p.). The skin was sterilized with a betadine wash solution. A transverse incision was then made in the skin of lower abdomen (approx. 1.0 cm in length) and then in the abdominal wall. The urinary bladder was isolated with a blunt forceps and the prostate identified under a dissection microscope. Cell suspension (1×10⁶ cells/30 μl of 1:1 PBS and Matrigel) was injected into the prostate with a 30 G needle to achieve a ballooning of the prostate to assure intraprostatic injection. The abdominal wall was then sutured close with absorbable sutures and the outer dermal layer clamped with wound clips. A single dose of buprenorphine was given S.C (1.0 mg/kg) immediately after the procedure for pain relief. All the animals recovered readily and were viable after the procedure. Wound Clips were removed after 1 week. Tumor growth was monitored weekly by palpitation and ultrasound. Animals were euthanized 8 week (TRAMP-C) or 4 week (Rv1) after intraprostatic injection, when the tumor size reached about 1 cm (TRAMP-C) or 2 to 3 cm (Rv1) in diameter. The GU tract was dissected and size of prostate tumors measured by a caliper. The tumor volume was calculated as length×width×thickness.

Blood tumor cell burden (intravasation assay). Measurement of tumor cells present in blood was performed as described (Qi et al., 2008). Nude mice with 4-week-old

Rvl allograft tumors were anesthetized with Avertin and blood was drawn from the right ventricle of the heart using a heparin-coated 22-gauge needle and a 1 ml syringe. Blood was added to a 10-cm plate containing Rvl cell growth medium and incubated overnight. 24 h later, plates were washed with PBS 3 times, and replaced with selection medium containing puromycin (1.0 μg/ml), allowing removal of blood cells and selection of puromycin-resistant colonies (transfected with ectopic expression vectors). The number of colonies was counted after two weeks.

Establish stable cell lines co-expressing 3 genes. The method for simultaneous co-expression of 3 genes in cells has been reported by Joan Massague's group (Minn et al., 2005): briefly, three expression vectors carrying the same drug resistace gene were co-packaged into retroviral particles, followed by their transduction into cells, drug selection and confirmation for co-expression. Genes encoding Hes6, Sox9 and Jmjd1a have been subcloned into pBabe hygro retroviral vectors, which were then (15 μg each) co-transfected into packaging cells using Lipofectamine 2000, thus packaging the 3 genes into viral particles. Virus-containing supernatants were harvested 48 and 96 h post-transfection.

TRAMP-C or CWR22Rv1 cells were first infected with pBabe puro retroviral vector encoding HA-PHYL or pKLO.1 lentiviral vector encoding FoxA2 shRNA, and selected with 1 μg/ml of puromycin to obtain stable clones. Expression of HA-PHYL or knockdown of FoxA2 was validated by western blot. Cells expressing PHYL or shFoxA2 were subsequently infected with viral particles containing Hes6, Sox9 and Jmjd1a, selected with 200 μg/ml of hygromycin B to obtain stable clones. Stable expression of the 3 genes was validated by quantitative RT-PCR.

Soft Agar Assay. TRAMP-C (1×10⁵) or Rv1 (1×10⁴) cells were mixed with agar to a final concentration of 0.4% and layered on top of 0.8% agar in 6-well plates. Triplicate plates were continuously treated with 1% O2 for 3 weeks and replaced with hypoxia-equalibriated fresh growth media every day in a hypoxia chamber (InVivo 400). The total number of colonies on each plate was counted after 3 weeks.

siRNA transfections. siRNAs purchased from Ambion were: Negative control siRNA, mouse HIF-1α siRNAs, mouse HIF-1β siRNAs, mouse FoxA2 siRNAs, mouse and human p300 siRNAs. 150 nM of siRNAs was transfected into 5 million cells using Amax Nucleofector according to the manufacturer's protocol. To effectively knock down the target genes, 3 respective specific siRNAs (50 nM each) were pooled for transfection. Cells were analyzed 48 or 72 h post-transfection.

Immunoprecipitation and Western blotting. For immunoprecipitation, cells were harvested in lysis buffer containing 50 mM Tris-HCl pH7.5, 150 mM NaCl, 0.5% NP40, 1 mM EDTA, 1 mM sodium orthovanadate, 1mM PMSF, 10 μg/ml aprotinin, 10 μg/ml leupeptine and 10 μg/ml pepstatin A. To immunoprecipitate Flag-tagged proteins, lysates were incubated with M2 beads (Sigma) overnight, the beads were washed 3 times, and precipitated proteins were eluted with 1 mg/ml of flag peptide. To immunoprecipitate endogenous proteins, cells lysates were incubated with 3 μg of primary antibodies overnight, and followed by incubation with Protein G beads for 1 h. After washing 3 times, the precipitated proteins were eluted with SDS loading buffer. To extract whole cell lysate, cells were harvested using RIPA buffer (50 mM Tris-HCl (pH7.5), 150 mM NaCl, 1% Triton X-100, 0.1% SDS, 0.1% Na-deoxycholate, 1 mM EDTA, 1 mM sodium orthovanadate, 1 mM PMSF, 10 μg/ml aprotinin, and 10 μg/ml leupeptine). Cell lysates were subjected to SDS-PAGE and proteins transferred onto a nitrocellulose membrane (Osmonics Inc.). The membrane was probed with primary antibodies followed by a secondary antibody conjugated with fluorescent dye and detected by the Odyssey detecting system (Amersham Bioscience).

In Vitro Binding Experiment. His-FoxA2 was purified from bacteria using Ni-NTA Agarose (Qiagen). Flag-HIF-1αs, Flag-p300, myc-FoxA2, HIF-1α and HIF-1β were translated in vitro using a TNT-coupled reticulocyte lysate system (Promega), in the presence or absence of ³⁵S. Ni-NTA agarose bound with his-FoxA2, or M2 beads bound with Flag-HIF-1α or Flag-p300 were incubated with other in vitro translated proteins at 4° C. for 2 h. The beads were extensively washed with PBS containing 0.5% NP40 and 150 mM NaCl three times before subjection to SDS-PAGE followed by autoradiography and western blotting.

Hypoxia treatment. Cells were exposed to hypoxia (1% O2) in a hypoxia work station (In Vivo 400; Ruskin Corp.) and then processed immediately on ice.

Luciferase assay. Cells in 12-well plates were transfected with 500 ng of HRE-Luc vector and 100 ng of β-Gal vector using lipofectamine in triplicate. When other co-transfection was performed, the amount of each plasmid used is 500 ng. After 24 h, cells were exposed to hypoxia (1% O2) for 10 h prior to collection of cell lysate using 200 μl of reporter lysis buffer (Promega). 10 μl of cell lysates were loaded onto a 96-well plate and luciferase activity was measured using a Veritas Microplate Luminometer (Turner Biosystems) according to manufacturers' instruction. β-Gal assay was used to normalize transfection efficiency. For the β-Gaassay, 10 μl of cell lysate was incubated in 250 μl of reaction buffer (100 mM NaH2PO4 (pH7.5), 0.1% O-Nitrophenyl-β-D-galactopyranoside (ONPG), 1.2 mM MgCl2 and 50 mM β-mercaptoethanol) at 37° C. for 20 min, and the reaction was stopped by addition of 750 μl of 1M Na2CO3. The OD at 420 nm measured with a spectrophotometer was used to reflect β-Gal activity. The luciferase activity of each sample was divided by the β-Gal activity to calculate the relative luciferase activity. Data were presented as mean±SD (n=3).

Laser capture microdissection (LCM). Tumor tissues were collected, and immediately imbedded in OCT and frozen in isopentane. 8-gm sections were cut with a cryostat at −20° C., and collected on membrane slides. Each slide was stained for hematoxylin and eosin (H&E), and pure NE tumor cells were isolated using a laser capture microdisection system (MMI) according to the manufacturers' protocol.

Chromatin immunoprecipitation (ChIP) assay. ChIP assay was performed using a Magna CHIP™ kit (Upstate). Triplicate biological samples were performed and analyzed. Briefly, cells (1.5×10⁷) were maintained in normoxia or hypoxia (1% O₂) for 6 h, and crosslinked using 1% formaldehyde for 10 min at RT. The crosslinking was stopped by 5M glycine. Cells were lysed and sonicated to get 200-1000 bp chromatin fragments. Chromatin was immunoprecipitated with 5 μg of antibodies and 20 μl of protein A/G magnetic beads in a total volume of 0.5 ml overnight at 4° C. After 4 washes, crosslinking of protein/DNA complex was reversed, DNA was purified using spin column, and subjected to ordinary PCR or QPCR analysis. The PCR primers for ChIP assay are as the following: mouse Hes6 HRE1: (SEQ ID NO:1) forward, 5′-caagaacgctggagcagag-3′ and (SEQ ID NO:2) reverse, 5′-tgtctagctggctttgtcct-3′; mouse Hes6 HRE2: (SEQ ID NO:3) forward, 5′-gcctgcaggaaccaagaata-3′ and (SEQ ID NO:4) reverse, 5′-agttcctccgcatcctcttt-3′; mouse Hes6 HRE3: (SEQ ID NO:5) forward, 5′-ctaagtggcaggaggtctgg-3′ and (SEQ ID NO:6) reverse, 5′-acatgtcaatgcaccgattg-3′; mouse Sox9: (SEQ ID NO:7) forward, 5′-gggttgtggagggtcctagt-3′ and (SEQ ID NO:8) reverse, 5′-tgtgaaccgatgtgtgtgtg-3′; mouse Jmjdla: (SEQ ID NO:9) forward, 5′-tcaaaatggcggacctagac-3′ and (SEQ ID NO:10) reverse, 5′-ggacagcactgggacgtg-3′; mouse VEGFA: (SEQ ID NO:11) forward, 5′-gccagactacacagtgcata-3′ and (SEQ ID NO:12) reverse, 5′-gcttatctgagcccttgtctg-3′; human Hes6: (SEQ ID NO:13) forward, 5′-aggggactagagggagatgg-3′ and (SEQ ID NO:14) reverse, 5′-ctgagtttcttccggactcg-3′; human Jmjdla: (SEQ ID NO:15) forward, 5′-tctcaatcccactttggagaa-3′ and (SEQ ID NO:16) reverse, 5′-taggctgctgggcgaaat-3′; human VEGFA: (SEQ ID NO:17) forward, 5′-tcagttccctggcaacatct-3′ and (SEQ ID NO:18) reverse, 5′- caccaagtttgtggagctga-3′.

Microarray analysis. TRAMP-C cells were transfected with pcDNA, or FoxA2, or HIF-1α, or HIF-1α+FoxA2 using Amax Nucleofector in duplicate. 48 hour post transfection, cells were treated with normoxia or 1% hypoxia for 12 h before isolation of total RNA. 500 ng of total RNA was used for synthesis of biotin-labeled cRNA using an RNA amplification kit (Ambion). The biotinylated cRNA is labeled by incubation with streptavidin-Cy3 to generate probe for hybridization with the Mouse-6 Expression BeadChip (Illumina) that represents 48K mouse gene transcripts. The BeadChips were analyzed using the manufacturers BeadArray Reader and collected primary data using the supplied Scanner software. Data analysis was done in three stages. First, expression intensities were calculated for each gene probed on the array for all hybridizations using illumina's Beadstudio#2 software. Second, intensity values were quality controlled and normalized: quality control was carried out by using the Beadstudio detection P-value set to <0.05 as a cutoff. This removed genes which were never detected in the arrays. After this step, the initial ˜46,000 genes were reduced to 26,000. All the arrays were then normalized using the normalize.quantiles routine from the Affy package in R-Bioconductor. This procedure accounted for any variation in hybridization intensity between the individual arrays. Finally, these normalized data were imported into GeneSpring and analyzed for differentially expressed genes. The groups of biological replicates were described to the software and significantly differentially expressed genes determined on the basis of the Welch t-tests and fold difference changes in expression level.

qRT-PCR analysis. Total RNA was extracted using a total RNA miniprep kit (Sigma) and digested with DNase I. cDNA was synthesized using oligo-dT and random hexomer primers for SYBR Green QPCR analysis. 18S rRNA was used as an internal control. Triplicate biological samples were used for the QPCR analysis. The PCR primers were designed using Primer3 and their specificity was checked using BLAST. The PCR products were limited to 100-200 bp. The primers used for QPCR analysis were as the following: mVEGFA: (SEQ ID NO:19) forward, 5′-atcttcaagccgtcctgtgt-3′ and (SEQ ID NO:20) reverse, 5′-gcattcacatctgctgtgct-3′; hVEGFA: (SEQ ID NO:21) forward, 5′-ggggaggaggaagaagagaa-3′, and (SEQ ID NO:22) reverse, 5′-acttggcatggtggaggtag-3′; mGlut-1: (SEQ ID NO:23) forward, 5′-aaacatggaaccaccgctac-3′, and (SEQ ID NO:24) reverse, 5′-aggccaacaggttcatcatc-3′; hGlut-1: (SEQ ID NO:25) forward, 5′-cttcactgtcgtgtcgctgt-3′, and (SEQ ID NO:26) reverse, 5′-ccaggacccacttcaaagaa-3′; mouse FoxA2: (SEQ ID NO:27) forward, 5′-taagcgagctaaagggagca-3′, and (SEQ ID NO:28) reverse, 5′-gtggttgaaggcgtaatggt-3′; human FoxA2: (SEQ ID NO:29) forward, 5′-ctacgccaacatgaactcca-3′, and (SEQ ID NO:30) reverse, 5′-gaggtccatgatccactggt-3′; mouse Hes6: (SEQ ID NO:31) forward, 5′-catcgatgccactgtctcag-3′, and (SEQ ID NO:32) reverse, 5′-cggtttagttcagcctctgg-3′; human Hes6: (SEQ ID NO:33) forward, 5′-ccctgaggctgaactgagtc-3′, and (SEQ ID NO:34) reverse, 5′-taccccaccacatctgaacc-3′; mouse Sox9: (SEQ ID NO:35) forward, 5′-cgactacgctgaccatcaga-3′, and (SEQ ID NO:36) reverse, 5′-agactggttgttcccagtgc-3′; human Sox9: (SEQ ID NO:37) 5′-atcaagacggagcagctgag-3′, and (SEQ ID NO:38) reverse, 5′-tggtggtcggtgtagtcgta-3′; mouse Jmjdla: (SEQ ID NO:39) forward, 5′-gagccacagtcggagacttc-3′, and (SEQ ID NO:40) reverse, 5′-ttggccatcagatcatcaaa-3′; mouse Plod2: (SEQ ID NO:41) forward, 5′-tccctcccaaggttacactg-3′, and (SEQ ID NO:42) reverse, 5′-gttcctggcttctgcttgac-3′; human Jmjdla: (SEQ ID NO:43) forward, 5′-caggagctccacatcaggtt-3′, and (SEQ ID NO:44) reverse, 5′-tgcatctttcactgcatggt-3′; mouse p300: (SEQ ID NO:45) forward, 5′-gaggagagaggccctgagtt-3′, and (SEQ ID NO:46) reverse, 5′-cggtaaagtgcctccaatgt-3′; human p300: (SEQ ID NO:47) forward, 5′-tcagccaagcggcctaaac-3′, and (SEQ ID NO:48) reverse, 5′-tcaccaccattggttagtccc-3′; mouse Siah2: (SEQ ID NO:49) forward, 5′-gtatgctaccacgggctgtt-3′, and (SEQ ID NO:50) reverse, 5′-atgcatgagatgggacatca-3′; mouse Siah1a: (SEQ ID NO:51) forward, 5′-ccagaaaggcaaggtagcag-3′, and (SEQ ID NO:52) reverse, 5′-gccccagacatttgaagaga-3′; 18S rRNA: (SEQ ID NO:53) forward, 5′-gagcgaaagcatttgccaag-3′, and (SEQ ID NO:54) reverse, 5′-ggcatcgtttatggtcggaa-3′.

Histological analyses. Tumors and organs collected from TRAMP mice were fixed in Z-fix (buffered zinc formalin fixatives, Anatech) overnight. After fixation, organs were washed twice with PBS and processed for paraffin embedding. Organs embedded in paraffin blocks were sliced at 5 μm and stained with hematoxylin and eosin or used for immunohistochemistry. H&E stained sections as well as sections that were subjected to IHC analysis were analyzed by two pathologists.

Cluster analysis of human prostate cancers. The Homo sapiens prostate cancer dataset (GSE3325) was downloaded from Gene Expression Ontology (website ncbi.nlm.nih.gov/geo/query/acc.cgi?acc=GSE3325). R-Bioconductor MAS was used to extract the data from the Affymetrix CEL files for the HG-U133 plus 2 arrays. The normalize.quantiles routine from the bioconductor affy package was used to normalize the arrays. The subset of genes and data of interest were extracted and clustered using GenePattern's (website genepattern.org) hierarchical clustering tool. The resulting matrix was displayed using the heatmap from GenePattern's hierarchical clustering viewer.

### REFERENCES

Ahmed, A. U., Schmidt, R. L., Park, C. H., Reed, N. R., Hesse, S. E., Thomas, C.

F., Molina, J. R., Deschamps, C., Yang, P., Aubry, M. C., and Tang, A. H. (2008). Effect of disrupting seven-in-absentia homolog 2 function on lung cancer cell growth. J Natl Cancer Inst 100, 1606-1629.

Amarillo, R., Viukov, S. V., Sharir, A., Eshkar-Oren, I., Johnson, R. S., and Zelzer, E. (2007). HIFlalpha regulation of Sox9 is necessary to maintain differentiation of hypoxic prechondrogenic cells during early skeletogenesis. Development 134, 3917-3928.

Aragones, J., Fraisl, P., Baes, M., and Carmeliet, P. (2009). Oxygen sensors at the crossroad of metabolism. Cell Metab 9, 11-22.

Arany, Z., Huang, L. E., Eckner, R., Bhattacharya, S., Jiang, C., Goldberg, M. A., Bunn, H. F., and Livingston, D. M. (1996). An essential role for p300/CBP in the cellular response to hypoxia. Proc Natl Acad Sci U S A 93, 12969-12973.

Beyer, S., Kristensen, M. M., Jensen, K. S., Johansen, J. V., and Staller, P. (2008). The Histone Demethylases JMJD1A and JMJD2B Are Transcriptional Targets of

Hypoxia-inducible Factor HIF. J Biol Chem 283, 36542-36552.

Bonkhoff, H. (1998). Neuroendocrine cells in benign and malignant prostate tissue: morphogenesis, proliferation, and androgen receptor status. Prostate Suppl 8, 18-22.

Chiaverotti, T., Couto, S. S., Donjacour, A., Mao, J. H., Nagase, H., Cardiff, R. D., Cunha, G. R., and Balmain, A. (2008). Dissociation of epithelial and neuroendocrine carcinoma lineages in the transgenic adenocarcinoma of mouse prostate model of prostate cancer. Am J Pathol 172, 236-246.

Cindolo, L., Cantile, M., Vacherot, F., Terry, S., and de la Taille, A. (2007). Neuroendocrine differentiation in prostate cancer: from lab to bedside. Urol Int 79, 287-296.

Cirillo, L. A., Lin, F. R., Cuesta, I., Friedman, D., Jarvik, M., and Zaret, K. S. (2002). Opening of compacted chromatin by early developmental transcription factors HNF3 (FoxA) and GATA-4. Mol Cell 9, 279-289.

Clark K L, Halay E D, Lai E, Burley S K 1993 Co-crystal structure of the HNF-3/fork head DNA-recognition motif resembles histone H5. Nature 364:412-420.

Deeble, P. D., Murphy, D. J., Parsons, S. J., and Cox, M. E. (2001). Interleukin-6- and cyclic AMP-mediated signaling potentiates neuroendocrine differentiation of LNCaP prostate tumor cells. Mol Cell Biol 21, 8471-8482.

Deng, X., Liu, H., Huang, J., Cheng, L., Keller, E. T., Parsons, S. J., and Hu, C. D. (2008). Ionizing radiation induces prostate cancer neuroendocrine differentiation through interplay of CREB and ATF2: implications for disease progression. Cancer Res 68, 9663-9670.

Emerling, B. M., Weinberg, F., Liu, J. L., Mak, T. W., and Chandel, N. S. (2008). PTEN regulates p300-dependent hypoxia-inducible factor 1 transcriptional activity through Forkhead transcription factor 3a (FOXO3a). Proc Natl Acad Sci USA 105, 2622-2627.

Erbel, P. J., Card, P. B., Karakuzu, O., Bruick, R. K., and Gardner, K. H. (2003). Structural basis for PAS domain heterodimerization in the basic helix—loop—helix-PAS transcription factor hypoxia-inducible factor. Proc Natl Acad Sci USA 100, 15504-15509.

Erler J T, et al.(2006) Lysyl oxidase is essential for hypoxia-induced metastasis. Nature 440:1222-1226.

Eun, B., Lee, Y., Hong, S., Kim, J., Lee, H. W., Kim, K., Sun, W., and Kim, H. (2008). Hes6 controls cell proliferation via interaction with cAMP-response element-binding protein-binding protein in the promyelocytic leukemia nuclear body. J Biol Chem 283, 5939-5949.

Foster, B. A., Gingrich, J. R., Kwon, E. D., Madias, C., and Greenberg, N. M. (1997). Characterization of prostatic epithelial cell lines derived from transgenic adenocarcinoma of the mouse prostate (TRAMP) model. Cancer Res 57, 3325-3330.

Frew, I. J., Hammond, V. E., Dickins, R. A., Quinn, J. M., Walkley, C. R., Sims, N. A., Schnall, R., Della, N. G., Holloway, A. J., Digby, M. R., et al. (2003). Generation and analysis of Siah2 mutant mice. Mol Cell Biol 23, 9150-9161.

Fukuba, H., Takahashi, T., Jin, H. G., Kohriyama, T., and Matsumoto, M. (2008). Abundance of aspargynyl-hydroxylase FIH is regulated by Siah-1 under normoxic conditions. Neurosci Lett 433, 209-214.

Gingrich, J. R., Barrios, R. J., Morton, R. A., Boyce, B. F., DeMayo, F. J., Finegold, M. J., Angelopoulou, R., Rosen, J. M., and Greenberg, N. M. (1996). Metastatic prostate cancer in a transgenic mouse. Cancer Res 56, 4096-4102.

Gordan, J. D., Bertout, J. A., Hu, C. J., Diehl, J. A., and Simon, M. C. (2007). HIF-2alpha promotes hypoxic cell proliferation by enhancing c-myc transcriptional activity. Cancer Cell 11, 335-347.

Gustafsson, M. V., Zheng, X., Pereira, T., Gradin, K., Jin, S., Lundkvist, J., Ruas, J. L., Poellinger, L., Lendahl, U., and Bondesson, M. (2005). Hypoxia requires notch signaling to maintain the undifferentiated cell state. Dev Cell 9, 617-628.

Hirano, D., Jike, T., Okada, Y., Minei, S., Sugimoto, S., Yamaguchi, K., Yoshikawa, T., Hachiya, T., Yoshida, T., and Takimoto, Y. (2005). Immunohistochemical and ultrastructural features of neuroendocrine differentiated carcinomas of the prostate: an immunoelectron microscopic study. Ultrastruct Pathol 29, 367-375.

House, C. M., Frew, I. J., Huang, H. L., Wiche, G., Traficante, N., Nice, E., Catimel, B., and Bowtell, D. D. (2003). A binding motif for Siah ubiquitin ligase. Proc Natl Acad Sci USA 100, 3101-3106.

Hu, Y., Ippolito, J. E., Garabedian, E. M., Humphrey, P. A., and Gordon, J. I. (2002). Molecular characterization of a metastatic neuroendocrine cell cancer arising in the prostates of transgenic mice. J Biol Chem 277, 44462-44474.

Huss, W. J., Gray, D. R., Tavakoli, K., Marmillion, M. E., Durham, L. E., Johnson, M. A., Greenberg, N. M., and Smith, G. J. (2007). Origin of androgen-insensitive poorly differentiated tumors in the transgenic adenocarcinoma of mouse prostate model. Neoplasia 9, 938-950.

Huss, W. J., Gregory, C. W., and Smith, G. J. (2004). Neuroendocrine cell differentiation in the CWR22 human prostate cancer xenograft: association with tumor cell proliferation prior to recurrence. Prostate 60, 91-97.

Ivan M,et al.(2001) HIFalpha targeted for VHL-mediated destruction by proline hydroxylation: implications for 02 sensing. Science 292:464-468.

Jaakkola P, et al.(2001) Targeting of HIF-alpha to the von Hippel-Lindau ubiquitylation complex by O2-regulated prolyl hydroxylation. Science 292:468-472.

Kaestner K H, Hiemisch H, Luckow B, Schutz G 1994 The HNF-3 gene family of transcription factors in mice: gene structure, cDNA sequence, and mRNA distribution. Genomics 20:377-385.

Kaidi, A., Williams, A. C., and Paraskeva, C. (2007). Interaction between beta-catenin and HIF-1 promotes cellular adaptation to hypoxia. Nat Cell Biol 9, 210-217.

Kasper, L. H., Boussouar, F., Boyd, K., Xu, W., Biesen, M., Rehg, J., Baudino, T. A., Cleveland, J. L., and Brindle, P. K. (2005). Two transactivation mechanisms cooperate for the bulk of HIF-1-responsive gene expression. EMBO J 24, 3846-3858.

Kim, J. Y., Kim, H. J., Kim, K. T., Park, Y. Y., Seong, H. A., Park, K. C., Lee, I. K., Ha, H., Shong, M., Park, S. C., and Choi, H. S. (2004). Orphan nuclear receptor small heterodimer partner represses hepatocyte nuclear factor 3/Foxa transactivation via inhibition of its DNA binding. Mol Endocrinol 18, 2880-2894.

Lai E, Prezioso V R, Smith E, Litvin O, Costa R H, Darnell Jr J E 1990 HNF-3A, a hepatocyte-enriched transcription factor of novel structure is regulated transcriptionally. Genes Dev 4:1427-1436.

Lai E, Prezioso V R, Tao W F, Chen W S, Darnell Jr J E 1991 Hepatocyte nuclear factor 3α belongs to a gene family in mammals that is homologous to the Drosophila homeotic gene fork head. Genes Dev 5:416-427.

Loh, Y. H., Zhang, W., Chen, X., George, J., and Ng, H. H. (2007). Jmjd1a and Jmjd2c histone H3 Lys 9 demethylases regulate self-renewal in embryonic stem cells. Genes Dev 21, 2545-2557.

Makino, Y., Kanopka, A., Wilson, W. J., Tanaka, H., and Poellinger, L. (2002). Inhibitory PAS domain protein (IPAS) is a hypoxia-inducible splicing variant of the hypoxia-inducible factor-3alpha locus. J Biol Chem 277, 32405-32408.

Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436,518-524.

Moller, A., House, C. M., Wong, C. S., Scanlon, D. B., Liu, M. C., Ronai, Z., and Bowtell, D. D. (2009). Inhibition of Siah ubiquitin ligase function. Oncogene 28, 289-296.

Monsef, N., Helczynski, L., Lundwall, A., and Pahlman, S. (2007). Localization of immunoreactive HIF-1alpha and HIF-2alpha in neuroendocrine cells of both benign and malignant prostate glands. Prostate 67, 1219-1229.

Nakayama K, et al. (2004) Siah2 regulates stability of prolyl-hydroxylases, controls HIF1alpha abundance, and modulates physiological responses to hypoxia. Cell 117:941-952.

Nakayama, K., Frew, I. J., Hagensen, M., Skals, M., Habelhah, H., Bhoumik, A., Kadoya, T., Erdjument-Bromage, H., Tempst, P., Frappell, P. B., et al. (2004). Siah2 regulates stability of prolyl-hydroxylases, controls HIF l alpha abundance, and modulates physiological responses to hypoxia. Cell 117, 941-952.

Nakayama, K., Qi, J., and Ronai, Z. (2009). The ubiquitin ligase Siah2 and the hypoxia response. Mol Cancer Res 7, 443-451.

Nowak, J. A., Polak, L., Pasolli, H. A., and Fuchs, E. (2008). Hair follicle stem cells are specified and function in early skin morphogenesis. Cell Stem Cell 3, 33-43.

Pennacchietti S, et al.(2003) Hypoxia promotes invasive growth by transcriptional activation of the met protooncogene. Cancer Cell 3:347-361.

Qi, J., Nakayama, K., Gaitonde, S., Goydos, J. S., Krajewski, S., Eroshkin, A., Bar-Sagi, D., Bowtell, D., and Ronai, Z. (2008). The ubiquitin ligase Siah2 regulates tumorigenesis and metastasis by HIF-dependent and -independent pathways. Proc Natl Acad Sci USA 105, 16713-16718.

Ruas, J. L., Berchner-Pfannschmidt, U., Malik, S., Gradin, K., Fandrey, J., Roeder, R. G., Pereira, T., and Poellinger, L. (2010). Complex regulation of the transactivation function of hypoxia-inducible factor-1 alpha by direct interaction with two distinct domains of the CREB-binding protein/p300. J Biol Chem 285, 2601-2609.

Schmidt, R. L., Park, C. H., Ahmed, A. U., Gundelach, J. H., Reed, N. R., Cheng, S., Knudsen, B. E., and Tang, A. H. (2007). Inhibition of RAS-mediated transformation and tumorigenesis by targeting the downstream E3 ubiquitin ligase seven in absentia homologue. Cancer Res 67, 11798-11810.

Sella, A., Konichezky, M., Flex, D., Sulkes, A., and Baniel, J. (2000). Low PSA metastatic androgen- independent prostate cancer. Eur Urol 38, 250-254.

Semenza G L (2003) Targeting HIF-1 for cancer therapy. Nat Rev Cancer 3:721-732.

Semenza, G. L. (2003). Targeting HIF-1 for cancer therapy. Nat Rev Cancer 3, 721-732.

Shah, R. B., Mehra, R., Chinnaiyan, A. M., Shen, R., Ghosh, D., Zhou, M., Macvicar, G. R., Varambally, S., Harwood, J., Bismar, T. A., et al. (2004). Androgen-independent prostate cancer is a heterogeneous group of diseases: lessons from a rapid autopsy program. Cancer Res 64, 9209-9216.

Shimizu, S., Kumagai, J., Eishi, Y., Uehara, T., Kawakami, S., Takizawa, T., and Koike, M. (2007). Frequency and number of neuroendocrine tumor cells in prostate cancer: no difference between radical prostatectomy specimens from patients with and without neoadjuvant hormonal therapy. Prostate 67, 645-652.

Sotomayor, P., Godoy, A., Smith, G. J., and Huss, W. J. (2008). Oct4A is expressed by a subpopulation of prostate neuroendocrine cells. Prostate 69, 401-410.

Sramkoski, R. M., Pretlow, T. G., 2nd, Giaconia, J. M., Pretlow, T. P., Schwartz, S., Sy, M. S., Marengo, S. R., Rhim, J. S., Zhang, D., and Jacobberger, J. W. (1999). A new human prostate carcinoma cell line, 22Rv1. In Vitro Cell Dev Biol Anim 35, 403-409.

Staller P, et al.(2003) Chemokine receptor CXCR4 downregulated by von Hippel-Lindau tumour suppressor pVHL. Nature 425:307-311.

Stiehl D P, et al.(2006) Increased prolyl 4-hydroxylase domain proteins compensate for decreased oxygen levels. Evidence for an autoregulatory oxygen-sensing system. J Biol Chem 281:23482-23491.

Tuckerman J R, et al.(2004) Determination and comparison of specific activity of the HIF-prolyl hydroxylases. FEBS Lett 576:145-150.

Vias, M., Massie, C. E., East, P., Scott, H., Warren, A., Zhou, Z., Nikitin, A. Y., Neal, D. E., and Mills, I. G. (2008). Pro-neural transcription factors as cancer markers. BMC Med Genomics 1, 17.

Wang, H., Leav, I., Ibaragi, S., Wegner, M., Hu, G. F., Lu, M. L., Balk, S. P., and Yuan, X. (2008). SOX9 is expressed in human fetal prostate epithelium and enhances prostate cancer invasion. Cancer Res 68, 1625-1630.

Weigel D, Jackie H 1990 The fork head domain: a novel DNA binding motif of eukaryotic transcription factors? Cell 63:455-456.

Wirthner R, et al.(2007) Determination and modulation of prolyl-4-hydroxylase domain oxygen sensor activity. Methods Enzymol 435:43-60.

Yuan, T. C., Veeramani, S., Lin, F. F., Kondrikou, D., Zelivianski, S., Igawa, T., Karan, D., Batra, S. K., and Lin, M. F. (2006). Androgen deprivation induces human prostate epithelial neuroendocrine differentiation of androgen-sensitive LNCaP cells. Endocr Relat Cancer 13, 151-167.

Zhou, Z., Flesken-Nikitin, A., Corney, D. C., Wang, W., Goodrich, D. W., Roy-Burman, P., and Nikitin, A. Y. (2006). Synergy of p53 and Rb deficiency in a conditional mouse model for metastatic prostate cancer. Cancer Res 66, 7889-7898.

1-3. (canceled)

4. The method of claim 49 further comprising determining if one or more of the cells that express FoxA2 and HIF-1α also express Hes6, Sox9, Jmjd1a, Plod2, or a combination.

5. The method of claim 4, wherein Hes6, Sox9, Jmjd1a, Plod2, or a combination are present at or above respective reference levels in the cells that express Hes6, Sox9, Jmjd1a, Plod2, or a combination.

6. The method of claim 49, wherein the cancer sample is a prostate sample, a lung sample, a pancreatic sample, or a merkel cell sample.

7. The method of claim 49, wherein the cancer is prostate cancer, lung cancer, pancreatic cancer, or merkel cell carcinoma.

8. The method of claim 49 further comprising treating the subject with a cancer treatment.

9. The method of claim 8, wherein the cancer treatment is a neuroendocrine differentiation (NED)-associated cancer treatment.

10. The method of claim 49, wherein the subject has prostate cancer, lung cancer, pancreatic cancer, or merkel cell carcinoma.

11. A method of treating a subject at risk of metastasis of cancer, the method comprising
administering to a subject a composition that inhibits expression of one or more HIF-1α:FoxA2-regulated genes.

12. The method of claim 52, wherein the composition comprises an inhibitor of HIF-1α.

13. The method of claim 12, wherein the inhibitor of HIF-1α comprises a Siah2 inhibitor.

14. The method of claim 13, wherein the Siah2 inhibitor is a PHYL peptide.

15. The method of claim 52, wherein the composition comprises an inhibitor of FoxA2.

16. The method of claim 15, wherein the inhibitor of FoxA2 is shRNA.

17. The method of claim 52, wherein the composition disrupts formation of HIF-1α:FoxA2 complex.

18. The method of claim 52, wherein the composition inhibits the formation of a HIF-1α:FoxA2 complex.

19. The method of claim 17, wherein the HIF-1α:FoxA2 complex comprises HIF-1α:FoxA2 interaction domain, wherein the composition competes for the HIF-1α:FoxA2 interaction domain.

20. The method of claim 52, wherein the composition reduces p300 recruitment.

21. The method of claim 52, wherein the composition inhibits p300.

22. The method of claim 52, wherein the HIF-1α:FoxA2-regulated genes are Hes6, Sox9, Jmjd1a, Plod2, or a combination.

23. The method of claim 52, wherein the composition comprises a compound and a pharmaceutically acceptable carrier.

24. The method of claim 23, wherein the composition comprises two or more different inhibitors of expression of HIF-1α:FoxA2-regulated genes.

25. The method of claim 52, wherein the composition comprises a vector.

26. The method of claim 52, wherein the subject is diagnosed with a NED-associated cancer prior to treatment.

27. The method of claim 52, wherein the subject has prostate cancer, lung cancer, pancreatic cancer, or merkel cell carcinoma.

28. The method of claim 52, wherein the subject has suffered from cellular hypoxia.

29. The method of claim 28, wherein the cellular hypoxia is mild cellular hypoxia.

30. The method of claim 1, wherein the cancer comprises one or more cells that express FoxA2 and HIF-1α.

31. The method of claim 11, wherein the risk of metastasis is indicated by one or more cancer cells that express FoxA2 and HIF-1α.

32. The method of claim 31, wherein the risk of metastasis is indicated by one or more of the cancer cells that express FoxA2 and HIF-1α also expressing Hes6, Sox9, Jmjd1a, Plod2, or a combination.

33. A method of identifying an inhibitor of HIF1α:FoxA2 function or complex formation, the method comprising
contacting a compound with HIF-1α or FoxA2;
assaying binding of the compound to HIF-1α or FoxA2; and
determining if the compound inhibits HIF-1α:FoxA2 function or complex formation.

34. The method of claim 33, wherein the compound is a peptide having at least 85% sequence identity to HIF-1α:FoxA2 interaction domain.

35-48. (canceled)

49. A method comprising
detecting one or more cells in a cancer sample from a subject that express FoxA2 and HIF-1α,
wherein detection of cells that express FoxA2 and HIF-1α indicates a risk of metastasis of cancer in the subject, a risk or the presence of neuroendocrine differentiation (NED)-associated cancer in the subject, a poor prognosis of the cancer in the subject, or a combination.

50. The method of claim 49, wherein FoxA2 and HIF-1α are both present at or above respective reference levels in the cells that express FoxA2 and HIF-1α, wherein the presence of FoxA2 and HIF-1α at or above the respective reference levels indicate a risk of metastasis of cancer in the subject, a risk or the presence of neuroendocrine differentiation (NED)-associated cancer in the subject, a poor prognosis of the cancer in the subject, or a combination.

51. The method of claim 49 further comprising
comparing the number of cells, fraction of cells, level of FoxA2, level of HIF-1α, or a combination in the cancer sample with the number of cells, fraction of cells, level of FoxA2, level of HIF-1α, or a combination in a second cancer sample from the same subject,
wherein the cancer sample is taken from the subject prior to or earlier during treatment of the subject, wherein the second cancer sample is taken from the subject following treatment of the subject or later during treatment of the subject than the cancer sample,
wherein a reduction in the number of cells, fraction of cells, level of FoxA2, level of HIF-1α, or a combination indicates that the treatment has reduced the risk of metastasis in the subject, indicates that the treatment has had a positive effect on the cancer, or both.

52. The method of claim 8, wherein the cancer treatment comprises administering to the subject a composition that inhibits expression of one or more HIF-1α:FoxA2-regulated genes.


SEQUENCE LISTING (54 sequences)

SEQ ID NO: 1 - DNA, 19 bp, Artificial Sequence
  Synthetic Primer
caagaacgctggagcagag

SEQ ID NO: 2 - DNA, 20 bp, Artificial Sequence
  Synthetic primer
tgtctagctggctttgtcct

SEQ ID NO: 3 - DNA, 20 bp, Artificial Sequence
  Synthetic primer
gcctgcaggaaccaagaata

SEQ ID NO: 4 - DNA, 20 bp, Artificial Sequence
  Synthetic primer
agttcctccgcatcctcttt

SEQ ID NO: 5 - DNA, 20 bp, Artificial Sequence
  Synthetic primer
ctaagtggcaggaggtctgg

SEQ ID NO: 6 - DNA, 20 bp, Artificial Sequence
  Synthetic primer
acatgtcaatgcaccgattg

SEQ ID NO: 7 - DNA, 20 bp, Artificial Sequence
  Synthetic primer
gggttgtggagggtcctagt

SEQ ID NO: 8 - DNA, 20 bp, Artificial Sequence
  Synthetic primer
tgtgaaccgatgtgtgtgtg

SEQ ID NO: 9 - DNA, 20 bp, Artificial Sequence
  Synthetic primer
tcaaaatggcggacctagac

SEQ ID NO: 10 - DNA, 18 bp, Artificial Sequence
  Synthetic primer
ggacagcactgggacgtg

SEQ ID NO: 11 - DNA, 20 bp, Artificial Sequence
  Synthetic primer
gccagactacacagtgcata

SEQ ID NO: 12 - DNA, 21 bp, Artificial Sequence
  Synthetic primer
gcttatctgagcccttgtctg

SEQ ID NO: 13 - DNA, 20 bp, Artificial Sequence
  Synthetic primer
aggggactagagggagatgg

SEQ ID NO: 14 - DNA, 20 bp, Artificial Sequence
  Synthetic primer
ctgagtttcttccggactcg

SEQ ID NO: 15 - DNA, 21 bp, Artificial Sequence
  Synthetic primer
tctcaatcccactttggagaa

SEQ ID NO: 16 - DNA, 18 bp, Artificial Sequence
  Synthetic primer
taggctgctgggcgaaat

SEQ ID NO: 17 - DNA, 20 bp, Artificial Sequence
  Synthetic primer
tcagttccctggcaacatct

SEQ ID NO: 18 - DNA, 20 bp, Artificial Sequence
  Synthetic primer
caccaagtttgtggagctga

SEQ ID NO: 19 - DNA, 20 bp, Artificial Sequence
  Synthetic primer
atcttcaagccgtcctgtgt

SEQ ID NO: 20 - DNA, 20 bp, Artificial Sequence
  Synthetic primer
gcattcacatctgctgtgct

SEQ ID NO: 21 - DNA, 20 bp, Artificial Sequence
  Synthetic primer
ggggaggaggaagaagagaa

SEQ ID NO: 22 - DNA, 20 bp, Artificial Sequence
  Synthetic primer
acttggcatggtggaggtag

SEQ ID NO: 23 - DNA, 20 bp, Artificial Sequence
  Synthetic primer
aaacatggaaccaccgctac

SEQ ID NO: 24 - DNA, 20 bp, Artificial Sequence
  Synthetic primer
aggccaacaggttcatcatc

SEQ ID NO: 25 - DNA, 20 bp, Artificial Sequence
  Synthetic primer
cttcactgtcgtgtcgctgt

SEQ ID NO: 26 - DNA, 20 bp, Artificial Sequence
  Synthetic primer
ccaggacccacttcaaagaa

SEQ ID NO: 27 - DNA, 20 bp, Artificial Sequence
  Synthetic primer
taagcgagctaaagggagca

SEQ ID NO: 28 - DNA, 20 bp, Artificial Sequence
  Synthetic primer
gtggttgaaggcgtaatggt

SEQ ID NO: 29 - DNA, 20 bp, Artificial Sequence
  Synthetic primer
ctacgccaacatgaactcca

SEQ ID NO: 30 - DNA, 20 bp, Artificial Sequence
  Synthetic primer
gaggtccatgatccactggt

SEQ ID NO: 31 - DNA, 20 bp, Artificial Sequence
  Synthetic primer
catcgatgccactgtctcag

SEQ ID NO: 32 - DNA, 20 bp, Artificial Sequence
  Synthetic primer
cggtttagttcagcctctgg

SEQ ID NO: 33 - DNA, 20 bp, Artificial Sequence
  Synthetic primer
ccctgaggctgaactgagtc

SEQ ID NO: 34 - DNA, 20 bp, Artificial Sequence
  Synthetic primer
taccccaccacatctgaacc

SEQ ID NO: 35 - DNA, 20 bp, Artificial Sequence
  Synthetic primer
cgactacgctgaccatcaga

SEQ ID NO: 36 - DNA, 20 bp, Artificial Sequence
  Synthetic primer
agactggttgttcccagtgc

SEQ ID NO: 37 - DNA, 20 bp, Artificial Sequence
  Synthetic primer
atcaagacggagcagctgag

SEQ ID NO: 38 - DNA, 20 bp, Artificial Sequence
  Synthetic primer
tggtggtcggtgtagtcgta

SEQ ID NO: 39 - DNA, 20 bp, Artificial Sequence
  Synthetic primer
gagccacagtcggagacttc

SEQ ID NO: 40 - DNA, 20 bp, Artificial Sequence
  Synthetic primer
ttggccatcagatcatcaaa

SEQ ID NO: 41 - DNA, 20 bp, Artificial Sequence
  Synthetic primer
tccctcccaaggttacactg

SEQ ID NO: 42 - DNA, 20 bp, Artificial Sequence
  Synthetic primer
gttcctggcttctgcttgac

SEQ ID NO: 43 - DNA, 20 bp, Artificial Sequence
  Synthetic primer
caggagctccacatcaggtt

SEQ ID NO: 44 - DNA, 20 bp, Artificial Sequence
  Synthetic primer
tgcatctttcactgcatggt

SEQ ID NO: 45 - DNA, 20 bp, Artificial Sequence
  Synthetic primer
gaggagagaggccctgagtt

SEQ ID NO: 46 - DNA, 20 bp, Artificial Sequence
  Synthetic primer
cggtaaagtgcctccaatgt

SEQ ID NO: 47 - DNA, 19 bp, Artificial Sequence
  Synthetic primer
tcagccaagcggcctaaac

SEQ ID NO: 48 - DNA, 21 bp, Artificial Sequence
  Synthetic primer
tcaccaccattggttagtccc

SEQ ID NO: 49 - DNA, 20 bp, Artificial Sequence
  Synthetic primer
gtatgctaccacgggctgtt

SEQ ID NO: 50 - DNA, 20 bp, Artificial Sequence
  Synthetic primer
atgcatgagatgggacatca

SEQ ID NO: 51 - DNA, 20 bp, Artificial Sequence
  Synthetic primer
ccagaaaggcaaggtagcag

SEQ ID NO: 52 - DNA, 20 bp, Artificial Sequence
  Synthetic primer
gccccagacatttgaagaga

SEQ ID NO: 53 - DNA, 20 bp, Artificial Sequence
  Synthetic primer
gagcgaaagcatttgccaag

SEQ ID NO: 54 - DNA, 20 bp, Artificial Sequence
  Synthetic primer
ggcatcgtttatggtcggaa